(19) United States
(12) Patent Application Publication
(10) Pub. No.: US 2004/0254126 A1
(43) Pub. Date: Dec. 16, 2004

(54) 11-12 Bicyclic erythromycin derivatives

(76) Inventors: Yao-Ling Qiu, Andover, MA (US); Ly Tam Phan, Quincy, MA (US); Zhigang Chen, Acton, MA (US); Tongzhu Liu, Auburndale, MA (US); Yat Sun Or, Watertown, MA (US)

Correspondence Address: ELMORE CRAIG, P.C., 209 MAIN STREET, N. CHELMSFORD, MA, 01863, US

(21) Appl. No.: 10/806,748

(22) Filed: Mar. 23, 2004

Related U.S. Application Data
(63) Continuation-in-part of application No. 10/455,219, filed on Jun. 5, 2003, now Pat. No. 6,790,835.
(63) Continuation-in-part of application No. 10/455,648, filed on Jun. 5, 2003, now Pat. No. 6,765,016.
(63) Continuation-in-part of application No. 10/455,001, filed on Jun. 5, 2003, now Pat. No. 6,716,820.
(63) Continuation-in-part of application No. 10/454,865, filed on Jun. 5, 2003, now Pat. No. 6,774,115.

(51) Int. Cl. A61K 31/7048; C07H 17/08

(52) U.S. Cl. 514/29; 536/7.4

(57) ABSTRACT

The present invention discloses compounds of formula I, or pharmaceutically acceptable salts, esters, or prodrugs thereof:

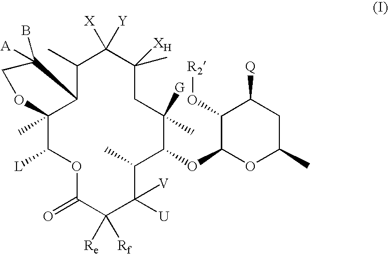

which exhibit antibacterial properties. The present invention further relates to pharmaceutical compositions comprising the aforementioned compounds for administration to a subject in need of antibiotic treatment. The invention also relates to methods of treating a bacterial infection in a subject by administering a pharmaceutical composition comprising the compounds of the present invention. The invention further includes process by which to make the compounds of the present invention.

### REFERENCE TO RELATED APPLICATIONS

This application is a continuation-in-part of U.S. application Ser. Nos. 10/455,219, 10/455,648, 10/455,001, and 10/454,865 (all filed on Jun. 5, 2003), all of which are incorporated herein by reference in their entirety.

### TECHNICAL FIELD

The present invention relates to novel semisynthetic macrolides having antibacterial activity and which are useful in the treatment and prevention of bacterial infections. More particularly, the invention relates to 11,12-cyclized erythromycin derivatives, compositions containing such compounds and methods for using the same, as well as processes for making such compounds.

### BACKGROUND OF THE INVENTION

Erythromycins A through D, represented by formula (E) as illustrated below,

| | |
|---|---|
| | (E) |
| 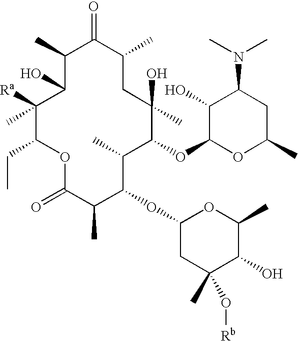 | |

| Erythromycin | R^{a} | R^{b} |
|---|---|---|
| A | —OH | —CH₃ |
| B | —H | —CH₃ |
| C | —OH | —H |
| D | —H | —H |

are well-known and potent antibacterial agents, used widely to treat and prevent bacterial infection. As with other antibacterials, however, bacterial strains having resistance or insufficient susceptibility to erythromycin have been identified. Also, erythromycin A has only weak activity against Gram-negative bacteria. Therefore, there is a continuing need to identify new erythromycin derivative compounds which possess improved antibacterial activity, which have less potential for developing resistance, which possess the desired Gram-negative activity, or which possess unexpected selectivity against target microorganisms. Consequently, numerous investigators have prepared chemical derivatives of erythromycin in an attempt to obtain analogs having modified or improved profiles of antibiotic activity.

Kashimura et al. have disclosed 6-O-methylerythromycin derivatives having a tricyclic basic nuclear structure in European Application 559896, published Nov. 11, 1991. Also, Asaka et al. have disclosed 5-O-desoaminylerythronolide derivatives containing a tricyclic carbamate structure in PCT Application WO 93/21200, published Apr. 22, 1992.

Recently erythromycin derivatives containing a variety of substituents at the 6-O position have been disclosed in U.S. Pat. Nos. 5,866,549, 6,075,011 and 6,420,555 B1 as well as PCT Applications WO 00/78773 and WO 03/024986. Furthermore, Ma et. al. have described erythromycin derivatives with aryl groups tethered to the C-6 position in J. Med Chem., 44, pp 41374156 (2001).

More recently, erythromycin derivatives containing a lactone moiety at the C11-C12 position have been disclosed in PCT Application WO 02/16380, published Feb. 28, 2002 as well as WO 02/50091 and WO 02/50092, both published Jun. 27, 2002 and WO 03/024986, which published Mar. 27, 2003.

### SUMMARY OF THE INVENTION

The present invention provides a novel class of C11-C12 bicyclic erythromycin derivatives that possess antibacterial activity.

In one aspect of the present invention there are disclosed novel bicyclic erythromycin compounds represented by the formulae illustrated below:

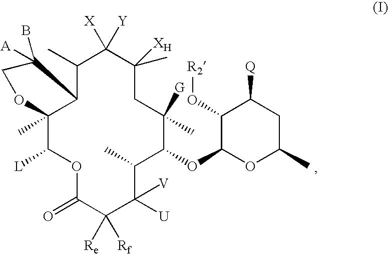

or their racemates, enantiomers, regioisomers, salts, esters or prodrugs thereof, wherein

A and B are independently selected from: halogen, NO₂, —CN, R₁, OR₁, S(O)ₙR₁, —NR₁C(O)R₂, —NR₁C(O)NR₃R₄, —NHS(O)ₙR₁, —C(O)NR₃R₄, —OC(O)NR₃R₄ and NR₃R₄;

Each R₁ and R₂ is independently selected from: hydrogen, deuterium, acyl, silane, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group;

Each of R₃ and R₄ is independently selected from: hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, a substituted or unsubstituted heterocyclic group; or can be taken together with the nitrogen atom to which they are attached to form a substituted or unsubstituted heterocyclic or heteroaromatic ring;

or A and B, taken together with the carbon atom to which they are attached, form a substituted or unsubstituted alicyclic, aromatic, heterocyclic or heteroaromatic ring;

or A and B, taken together with the carbon atom to which they are attached, are selected from: CO, C═CR₁R₂, C═NR₁, C═NOR₁, C═NO(CH₂)ₘR₁, C═NNHR₁, C═NNHCOR₁, C═NNHCONR₃R₄, C═NNHS(O)ₙR₁, or C═N—N═CR₁R₂;

L is selected from hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group;

G is independently selected from hydrogen, —CN or OR₁;

one of U or V is hydrogen and the other is independently selected from: R₁, OR₁, OC(O)R₁, OC(O)NR₃R₄, S(O)ₙR₁,

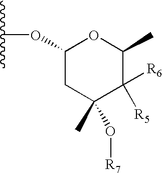

or other carbohydrate or sugar moiety;

one of R₅ or R₆ is hydrogen and the other is selected from: R₁, OR₁, or NR₃R₄;

or R₅ and R₆, taken together with the carbon atom to which they are attached, are selected from: C═O, C═C(R₁)₂, C═NR₁, C═C(R₁)₂, C═NOR₁, C═NO(CH₂)ₘR₁, C═NNR₃R₄, C═NNHCOR₁, C═NNHCONR₃R₄, C═NNHS(O)ₙR₁, or C═N—N═C(R₁)₂;

R₇ is independently selected from hydrogen or methyl;

or U and V, taken together with the carbon atom to which they are attached, are CO;

or UV and RₑR_{f}, taken together with the carbon atoms to which they are attached, are —C(R₁)═CH—;

one of Rₑ and R_{f} is selected from hydrogen or methyl, and the other is independently selected from halogen, deuterium, or R₁.

Q is NR₃R₄;

one of X and Y is hydrogen, substituted or unsubstituted aliphatic, and the other is independently selected from: hydroxy, —SH, —NH₂, or —NR₁H;

or X and Y, taken together with the carbon atom to which they are attached, are selected from: C═O, C═C(R₁)₂, C═NR₁, C═NOR₁, C═NO(CH₂)ₘR₁, C═NNHR₁, C═NNHCOR₁, C═NNHCONR₃R₄, C═NNHS(O)ₙR₁, or C═N—N═C(R₁)₂;

R₂′ and Rₚ are independently selected from hydrogen or a hydroxy protecting group;

X_{H} is selected from hydrogen or halogen;

m is an integer; and

n is 0, 1, or 2.

In an alternate embodiment of the present invention are compounds of formula II:

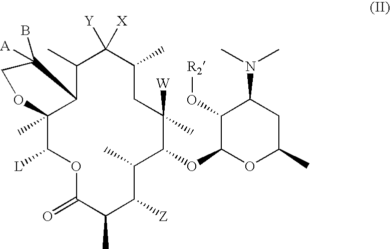

as well as pharmaceutically acceptable salts, esters and prodrugs thereof.

In formula II:

A is selected from:

(a) —OH;

(b) —ORₚ, where Rₚ is a hydroxy protecting group;

(c) —R₁, where R₁ is selected from:

1. aryl;

2. substituted aryl;

3. heteroaryl; and

4. substituted heteroaryl;

(d) —OR₁, where R₁ is as previously defined;

(e) —R₂, where R₂ is selected from:

1. hydrogen;

2. halogen;

3. C₁-C₆ alkyl containing 0, 1, 2, or 3 heteroatoms selected from O, S and N, optionally substituted with one or more substituents selected from halogen, cyano, oxo, aryl, substituted aryl, heteroaryl and substituted heteroaryl;

4. C₂-C₆ alkenyl containing 0, 1, 2, or 3 heteroatoms selected from O, S and N, optionally substituted with one or more substituents selected from halogen, cyano, oxo, aryl, substituted aryl, heteroaryl and substituted heteroaryl; and

5. C₂-C₆ alkynyl containing 0, 1, 2, or 3 heteroatoms selected from O, S and N, optionally substituted with one or more substituents selected from halogen, cyano, oxo, aryl, substituted aryl, heteroaryl and substituted heteroaryl;

(f) —OR₂, where R₂ is previously defined;

(g) —S(O)ₙR₁₁, where n=0, 1 or 2, and R₁₁ is selected from hydrogen, R₁ and R₂, where R₁ and R₂ are as previously defined

(h) —OC(O)R₁₁, where R₁₁ is as previously defined;

(i) —C(O)R₁₁, where R₁₁ is as previously defined;

(j) —C(O)NHR₁₁, where R₁₁ is as previously defined;

(k) —OC(O)NHR₁₁, where R₁₁ is as previously defined;

(l) —NHC(O)R₁₁, where R₁₁ is as previously defined;

(m)—NHC(O)NHR₁₁, where R₁₁ is as previously defined;

(n) —NHS(O)ₙR₁₁, where n and R₁₁ are as previously defined;

(o) —NR₁₄R₁₅, where R₁₄ and R₁₅ are each independently R₁₁, where R₁₁, is as previously defined; and

(p) —NHR₃, where R₃ is an amino protecting group;

B is selected from:

(a) hydrogen;

(b) deuterium;

(c) —CN;

(d) —NO₂;

(e) halogen;

(f) —OH;

(g) —R₁, where R₁ is as previously defined;

(h) —R₂, where R₂ is as previously defined; and

(i) —ORₚ, where Rₚ is as previously defined;

provided that when B is halogen, —NO₂, —OH or ORₚ, A is R₁ or R₂;

or, alternatively, A and B taken together with the carbon atom to which they are attached are selected from:

(a) C═O;

(b) C(OR₂)₂, where R₂ is as previously defined;

(c) C(SR₂)₂, where R₂ is as previously defined;

(d) C(OR₁₂)(OR₁₃), where R₁₂ and R₁₃ taken together are —(CH₂)ₘ—, and where m is 2 or 3;

(e) C(SR₁₂)(SR₁₃), where R₁₂ and R₁₃ taken together are —(CH₂)ₘ and, where m is as previously defined,

(f) C═CR₁₁R₁₄, where R₁₁ and R₁₄ are as previously defined;

(g) C═N—O—R₁₁, where R₁₁ is as previously defined;

(h) C═NNHR₁₁, where R₁₁ is as previously defined;

(i) C═NNHC(O)R₁₁, where R₁₁ is as previously defined;

(j) C═NN═CR₁₁R₁₄, where R₁₁ and R₁₄ are as previously defined;

(k) C═NNHC(O)NHR₁₁, where R₁₁ is as previously defined;

(l) C═NNHS(O)ₙR₁₁, where n and R₁₁ are as previously defined;

(m)C═NNHR₃, where R₃ is as previously defined; and

(n) C═NR₁₁, where R₁₁ is as previously defined;

one of X and Y is hydrogen and the other is selected from:

(a) hydrogen;

(b) deuterium;

(c) —OH;

(d) —ORₚ, where Rₚ is as previously defined; and

(e) —NR₄R₅, where R₄ and R₅ are each independently selected from:

1. hydrogen; and

2. C₁-C₁₂ alkyl, optionally substituted with one or more substituents selected from halogen, cyano, aryl, substituted aryl, heteroaryl and substituted heteroaryl; or

R₄ and R₅, taken together with the nitrogen atom to which they are attached form a 3-10 membered heteroalkyl ring containing 0-2 additional hetero atoms selected from O, S and N; or

alternatively, X and Y taken together with the carbon atom to which they are attached are selected from:

(a) C═O;

(b) C═NR₁₁, where R₁₁ is as previously defined;

(c) C═NC(O)R₁₁, where R₁₁ is as previously defined;

(d) C═N—OR₆, where R₆ is selected from:

1. hydrogen;

2. —CH₂O(CH₂)₂OCH₃,

3. —CH₂O(CH₂O)ₙCH₃, where n is as previously defined;

4. —C₁-C₁₂ alkyl, optionally substituted with one or more substituents selected from halogen, cyano, aryl, substituted aryl, heteroaryl and substituted heteroaryl;

5. C₃-C₁₂ cycloalkyl;

6. C(O)—C₁-C₁₂ alkyl;

7. C(O)—C₃-C₁₂ cycloalkyl;

8. C(O)—R₁, where R₁ is as previously defined; and

9. —Si(Rₐ)(R_{b})(R_{c}), wherein Rₐ, R_{b} and R_{c} are each independently selected from C₁-C₁₂ alkyl, aryl and substituted aryl; and

(e) C═N—O—C(R₇)(R₈)—O—R₆, where R₆ is as previously defined, provided that R₆ is not C(O)—C₁-C₁₂ alkyl, C(O)—C₃-C₁₂ cycloalkyl, or C(O)—R₁; and R₇ and R₈ taken together with the carbon atom to which they are attached form a C₃-C₁₂ cycloalkyl group or each is independently selected from:

1. hydrogen; and

2. C₁-C₁₂ alkyl;

L is selected from:

(a) —CH(OH)CH₃;

(b) C₁-C₆ alkyl, optionally substituted with one or more substituents selected from halogen, cyano, aryl, substituted aryl, heteroaryl and substituted heteroaryl;

(c) C₂-C₆ alkenyl, optionally substituted with one or more substituents selected from halogen, cyano, aryl, substituted aryl, heteroaryl and substituted heteroaryl; and

(d) C₂-C₆ alkynyl, optionally substituted with one or more substituents selected from halogen, cyano, aryl, substituted aryl, heteroaryl and substituted heteroaryl;

W is selected from:

(a) hydrogen;

(b) —OH;

(c) —CN;

(d) —OR₁₀, where R₁₀ is methyl, optionally substituted with one or more substituents selected from:

1. halogen;

2. deuterium;

3. —CN;

4. —R₁, where R₁ is as previously defined;

5. —OR₁₁, where R₁₁ is as previously defined;

6. —S(O)ₙR₁₁, where n and R₁₁ are as previously defined;

7. —OC(O)R₁₁, where R₁₁ is as previously defined;

8. —C(O)R₁₁, where R₁₁ is as previously defined;

9. —C(O)O R₁₁, where R₁₁ is as previously defined;

10. —C(O)NHR₁₁, where R₁₁ is as previously defined;

11. —OC(O)NHR₁₁, where R₁₁ is as previously defined;

12. —NHC(O)R₁₁, where R₁₁ is as previously defined;

13. —NHC(O)NHR₁₁, where R₁₁ is as previously defined; and

14. —NHS(O)ₙR₁₁, where n and R₁₁ are as previously defined; and

(e) —OC(O)NHR₁₁, where R₁₁ is as previously defined;

Z is selected from:

(a) hydrogen;

(b) —OH;

(c) —ORₚ, where Rₚ is as previously defined;

(d) —OR₁₁, where R₁₁ is as previously defined;

(e) —OC(O)R₁₁, where R₁₁ is as previously defined;

(f) —OC(O)NHR₁₁, where R₁₁ is as previously defined;

(g) —S(O)ₙR₁₁, where n and R₁₁ are as previously defined;

(h) —

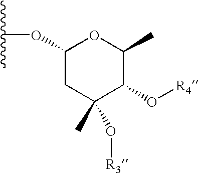

where R₃″ is hydrogen or methyl; R₄″ is hydrogen or Rₚ, where Rₚ is as previously defined; and

R₂′ is hydrogen or Rₚ, where Rₚ is as previously defined.

In yet another alternate embodiment of the present invention are compounds of formula III:

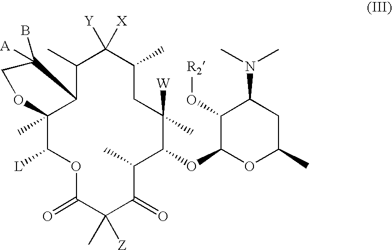

as well as pharmaceutically acceptable salts, esters and prodrugs thereof.

In formula III:

A is selected from:

(a) —OH;

(b) —ORₚ, where Rₚ is a hydroxy protecting group;

(c) —R₁, where R₁ is selected from:

1. aryl;

2. substituted aryl;

3. heteroaryl; and

4. substituted heteroaryl;

(d) —OR₁, where R₁ is as previously defined;

(e) —R₂, where R₂ is selected from:

1. hydrogen;

2. halogen;

3. C₁-C₆ alkyl containing 0, 1, 2, or 3 heteroatoms selected from O, S and N, optionally substituted with one or more substituents selected from halogen, cyano, oxo, aryl, substituted aryl, heteroaryl and substituted heteroaryl;

4. C₂-C₆ alkenyl containing 0, 1, 2, or 3 heteroatoms selected from O, S and N, optionally substituted with one or more substituents selected from halogen, cyano, oxo, aryl, substituted aryl, heteroaryl and substituted heteroaryl; and

5. C₂-C₆ alkynyl containing 0, 1, 2, or 3 heteroatoms selected from O, S and N, optionally substituted with one or more substituents selected from halogen, cyano, oxo, aryl, substituted aryl, heteroaryl and substituted heteroaryl;

(f) —OR₂, where R₂ is previously defined;

(g) —S(O)ₙR₁₁, where n=0, 1 or 2, and R₁₁ is selected from hydrogen, R₁ and R₂, where R₁ and R₂ are as previously defined

(h) —OC(O)R₁₁, where R₁₁ is as previously defined;

(i) —C(O)R₁₁, where R₁₁ is as previously defined;

(j) —C(O)NHR₁₁, where R₁₁ is as previously defined;

(k) —OC(O)NHR₁₁, where R₁₁ is as previously defined;

(l) —NHC(O)R₁₁, where R₁₁ is as previously defined;

(m)—NHC(O)NHR₁₁, where R₁₁ is as previously defined;

(n) —NHS(O)ₙR₁₁, where n and R₁₁ are as previously defined;

(o) —NR₁₄R₁₅, where R₁₄ and R₁₅ are each independently R₁₁, where R₁₁ is as previously defined; and

(p) —NHR₃, where R₃ is an amino protecting group;

B is selected from:

(a) hydrogen;

(b) deuterium;

(c) —CN;

(d) —NO₂;

(e) halogen;

(f) —OH;

(g) —R₁, where R₁ is as previously defined;

(h) —R₂, where R₂ is as previously defined; and

(i) —ORₚ, where Rₚ is as previously defined;

provided that when B is halogen, —NO₂, —OH or ORₚ, A is R₁ or R₂;

or, alternatively, A and B taken together with the carbon atom to which they are attached are selected from:

a) C═O;

b) C(OR₂)₂, where R₂ is as previously defined;

c) C(SR₂)₂, where R₂ is as previously defined;

d) C(OR₁₂)(OR₁₃), where R₁₂ and R₁₃ taken together are —(CH₂)ₘ—, and where m is 2or3;

e) C(SR₁₂)(SR₁₃), where R₁₂ and R₁₃ taken together are —(CH₂)ₘ and, where m is as previously defined,

f) C═CR₁₁R₁₄, where R₁₁ and R₁₄ are as previously defined;

g) C═N—O—R₁₁, where R₁₁ is as previously defined;

h) C═NNHR₁₁, where R₁₁ is as previously defined;

i) C═NNHC(O)R₁₁, where R₁₁ is as previously defined;

j) C═NN═CR₁₁R₁₄, where R₁₁ and R₁₄ are as previously defined;

k) C═NNHC(O)NHR₁₁, where R₁₁ is as previously defined;

l) C═NNHS(O)ₙR₁₁, where n and R₁₁ are as previously defined;

m) C═NNHR₃, where R₃ is as previously defined; and

n) C═NR₁₁, where R₁₁ is as previously defined;

one of X and Y is hydrogen and the other is selected from:

(a) hydrogen;

(b) deuterium;

(c) —OH;

(d) —ORₚ, where Rₚ is as previously defined; and

(e) —NR₄R₅, where R₄ and R₅ are each independently selected from:

1. hydrogen; and

2. C₁-C₁₂ alkyl, optionally substituted with one or more substituents selected from halogen, cyano, aryl, substituted aryl, heteroaryl and substituted heteroaryl; or

R₄ and R₅, taken together with the nitrogen atom to which they are attached form a 3-10 membered heteroalkyl ring containing 0-2 additional hetero atoms selected from O, S and N; or

alternatively, X and Y taken together with the carbon atom to which they are attached are selected from:

(a) C═O;

(b) C═NR₁₁, where R₁₁ is as previously defined;

(c) C═NC(O)R₁₁, where R₁₁ is as previously defined;

(d) C═N—OR₆, where R₆ is selected from:

1. hydrogen;

2. —CH₂O(CH₂)₂OCH₃,

3. —CH₂O(CH₂O)ₙCH₃, where n is as previously defined;

4. —C₁-C₁₂ alkyl, optionally substituted with one or more substituents selected from halogen, cyano, aryl, substituted aryl, heteroaryl and substituted heteroaryl;

5. C₃-C₁₂ cycloalkyl;

6. C(O)—C₁-C₁₂ alkyl;

7. C(O)—C₃-C₁₂ cycloalkyl;

8. C(O)—R₁, where R₁ is as previously defined; and

9. —Si(Rₐ)(R_{b})(R_{c}), wherein Rₐ, R_{b} and R_{c} are each independently selected from C₁-C₁₂ alkyl, aryl and substituted aryl; and

(e) C═N—O—C(R₇)(R₈)—O—R₆, where R₆ is as previously defined, provided that R₆ is not C(O)—C₁-C₁₂ alkyl, C(O)—C₃-C₁₂ cycloalkyl, or C(O)—R₁; and R₇ and R₈ taken together with the carbon atom to which they are attached form a C₃-C₁₂ cycloalkyl group or each is independently selected from:

1. hydrogen; and

2. C₁-C₁₂ alkyl;

L is selected from:

(a) —CH(OH)CH₃;

(b) C₁-C₆ alkyl, optionally substituted with one or more substituents selected from halogen, cyano, aryl, substituted aryl, heteroaryl, and substituted heteroaryl;

(c) C₂-C₆ alkenyl, optionally substituted with one or more substituents selected from halogen, cyano, aryl, substituted aryl, heteroaryl, and substituted heteroaryl; and

(d) C₂-C₆ alkynyl, optionally substituted with one or more substituents selected from halogen, cyano, aryl, substituted aryl, heteroaryl, and substituted heteroaryl;

W is selected from:

(a) hydrogen;

(b) —OH;

(c) —CN;

(d) —OR₁, where R₁₀ is methyl, optionally substituted with one or more substituents selected from:

1. halogen;

2. deuterium;

3. —CN;

4. —R₁, where R₁ is as previously defined;

35. —OR₁₁, where R₁₁ is as previously defined;

6. —S(O)ₙR₁₁, where n and R₁₁ are as previously defined;

7. —OC(O)R₁₁, where R₁₁ is as previously defined;

8. —C(O)R₁₁, where R₁₁ is as previously defined;

9. —C(O)O R₁₁, where R₁₁ is as previously defined;

10. —C(O)NHR₁₁, where R₁₁ is as previously defined;

11. —OC(O)NHR₁₁, where R₁₁ is as previously defined;

12. —NHC(O)R₁₁, where R₁₁ is as previously defined;

13. —NHC(O)NHR₁₁, where R₁₁ is as previously defined; and

14. —NHS(O)ₙR₁₁, where n and R₁₁ are as previously defined; and

(e) —OC(O)NHR₁₁, where R₁₁ is as previously defined;

Z is selected from:

(a) hydrogen;

(b) halogen; and

(c) C₁-C₆ alkyl, optionally substituted with one or more substituents selected from halogen, cyano, aryl, substituted aryl, heteroaryl and substituted heteroaryl; and

R₂′ is hydrogen or Rₚ, where Rₚ, is as previously defined.

A further alternate embodiment of the present invention are compounds of formula IV:

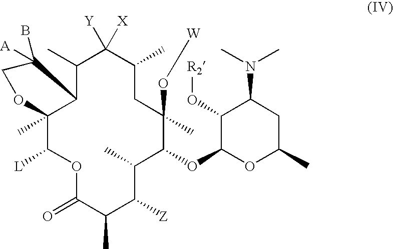

as well as pharmaceutically acceptable salts, esters and prodrugs thereof.

In formula IV:

A is selected from:

(a) —OH;

(b) —ORₚ, where Rₚ is a hydroxy protecting group;

(c) —R₁, where R₁ is selected from:

1. aryl;

2. substituted aryl;

3. heteroaryl; and

4. substituted heteroaryl;

(d) —OR₁, where R₁ is as previously defined;

(e) —R₂, where R₂ is selected from:

1. hydrogen;

2. halogen;

3. C₁-C₆ alkyl containing 0, 1, 2, or 3 heteroatoms selected O, S and N, optionally substituted with one or more substituents selected from halogen, cyano, oxo, aryl, substituted aryl, heteroaryl, and substituted heteroaryl;

4. C₂-C₆ alkenyl containing 0, 1, 2, or 3 heteroatoms selected from O, S and N, optionally substituted with one or more substituents selected from halogen, cyano, oxo, aryl, substituted aryl, heteroaryl, and substituted heteroaryl; and

5. C₂-C₆ alkynyl containing 0, 1, 2, or 3 heteroatoms selected from O, S and N, optionally substituted with one or more substituents selected from halogen, cyano, oxo, aryl, substituted aryl, heteroaryl, and substituted heteroaryl;

(f) —OR₂, where R₂ is previously defined;

(g) —S(O)ₙR₁₁, where n=0, 1 or 2, and R₁₁ is selected from hydrogen, R₁ and R₂, where R₁ and R₂ are as previously defined;

(h) —OC(O) R₁₁, where R₁₁ is as previously defined;

(i) —C(O)R₁₁, where R₁₁ is as previously defined;

(j) —C(O)NHR₁₁, where R₁₁ is as previously defined;

(k) —OC(O)NHR₁₁, where R₁₁ is as previously defined;

(l) —NHC(O)R₁₁, where R₁₁ is as previously defined;

(m)—NHC(O)NHR₁₁, where R₁₁ is as previously defined;

(n) —NHS(O)ₙR₁₁, where n and R₁₁ are as previously defined;

(o) —NR₁₄R₁₅, where R₁₄ and R₁₅ are each independently R₁₁, where R₁₁ is as previously defined; and

(p) —NHR₃, where R₃ is an amino protecting group;

B is selected from:

(a) hydrogen;

(b) deuterium;

(c) —CN;

(d) —NO₂;

(e) halogen;

(f) —OH;

(g) —R₁, where R₁ is as previously defined;

(h) —R₂, where R₂ is as previously defined; and

(i) —ORₚ, where Rₚ is as previously defined;

provided that when B is halogen, —NO₂, —OH or ORₚ, A is R₁ or R₂;

or, alternatively, A and B taken together with the carbon atom to which they are attached are selected from:

(a) C═O;

(b) C(OR₂)₂, where R₂ is as previously defined;

(c) C(SR₂)₂, where R₂ is as previously defined;

(d) C(OR₁₂)(OR₁₃), where R₁₂ and R₁₃ taken together are —(CH₂)ₘ—, and where m=2or3;

(e) C(SR₁₂)(SR₁₃), where R₁₂ and R₁₃ taken together are —(CH₂)ₘ— and where m is as previously defined,

(f) C═CR₁₁R₁₄, where R₁₁ and R₁₄ are as previously defined;

(g) C═N—O—R₁₁, where R₁₁ is as previously defined;

(h) C═NNHR₁₁, where R₁₁ is as previously defined;

(i) C═NNHC(O)R₁₁, where R₁₁ is as previously defined;

(j) C═NN═CR₁₁R₁₄, where R₁₁ and R₁₄ are as previously defined;

(k) C═NNHC(O)NHR₁₁, where R₁₁ is as previously defined;

(l) C═NNHS(O)ₙR₁₁, where n and R₁₁ are as previously defined;

(m)C═NNHR₃, where R₃ is as previously defined; and

(n) C═NR₁₁, where R₁₁ is as previously defined;

one of X and Y is hydrogen and the other is selected from:

(a) hydrogen;

(b) deuterium;

(c) —OH;

(d) —ORₚ, where Rₚ is as previously defined; and

(e) —NR₄R₅, where R₄ and R₅ are each independently selected from:

1. hydrogen; and

2. C₁-C₁₂ alkyl, optionally substituted with one or more substituents selected from halogen, cyano, aryl, substituted aryl, heteroaryl and substituted heteroaryl; or

R₄ and R₅, taken together with the nitrogen atom to which they are attached form a 3-10 membered heteroalkyl ring containing 0-2 additional hetero atoms selected from O, S and N; or

alternatively, X and Y taken together with the carbon atom to which they are attached are selected from:

(a) C═O;

(b) C═NR₁₁, where R₁₁ is as previously defined;

(c) C═NC(O)R₁₁, where R₁₁ is as previously defined;

(d) C═N—OR₆, where R₆ is selected from:

1. hydrogen;

2. —CH₂O(CH₂)₂OCH₃,

3. —CH₂O(CH₂O)ₙCH₃, where n is as previously defined;

4. —C₁-C₁₂ alkyl, optionally substituted with one or more substituents selected from the group consisting of halogen, cyano, aryl, substituted aryl, heteroaryl and substituted heteroaryl;

5. C₃-C₁₂ cycloalkyl;

6. C(O)C₁-C₁₂ alkyl;

7. C(O)—C₃-C₁₂ cycloalkyl;

8. C(O)—R₁, where R₁ is as previously defined; and

9. —Si(Rₐ)(R_{b})(R_{c}), wherein Rₐ, R_{b} and R_{c} are each independently selected from C₁-C₁₂ alkyl, aryl and substituted aryl; and

(e) C═N—O—C(R₇)(R₈)—O—R₆, where R₆ is as previously defined, provided that R₆ is not C(O)—C₁-C₁₂ alkyl, C(O)—C₃-C₁₂ cycloalkyl, or C(O)—R₁; and R₇ and R₈ taken together with the carbon atom to which they are attached form a C₃-C₁₂ cycloalkyl group or each is independently selected from:

1. hydrogen; and

2. C₁-C₁₂ alkyl;

L is selected from:

(a) —CH(OH)CH₃;

(b) C₁-C₆ alkyl, optionally substituted with one or more substituents selected from halogen, cyano, aryl, substituted aryl, heteroaryl, and substituted heteroaryl;

(c) C₂-C₆ alkenyl, optionally substituted with one or more substituents selected from halogen, cyano, aryl, substituted aryl, heteroaryl, and substituted heteroaryl; and

(d) C₂-C₆ alkynyl, optionally substituted with one or more substituents selected from halogen, cyano, aryl, substituted aryl, heteroaryl, and substituted heteroaryl;

W is selected from:

(a) C₂-C₆ alkyl containing 0, 1, 2, or 3 heteroatoms selected from O, S and N, optionally substituted with one or more substituents selected from halogen, cyano, oxo, aryl, substituted aryl, heteroaryl, and substituted heteroaryl;

(b) C₂-C₆ alkenyl containing 0, 1, 2, or 3 heteroatoms selected from O, S and N, optionally substituted with one or more substituents selected from halogen, cyano, oxo, aryl, substituted aryl, heteroaryl, and substituted heteroaryl; and

(c) C₂-C₆ alkynyl containing 0, 1, 2, or 3 heteroatoms selected from O, S and N, optionally substituted with one or more substituents selected from halogen, cyano, oxo, aryl, substituted aryl, heteroaryl, and substituted heteroaryl;

Z is selected from:

(a) hydrogen;

(b) —OH;

(c) —ORₚ, where Rₚ is as previously defined;

(d) —OR₁₁, where R₁₁ is as previously defined;

(e) —OC(O)R₁₁, where R₁₁ is as previously defined;

(f) —OC(O)NHR₁₁, where R₁₁ is as previously defined;

(g) —S(O)ₙR₁₁, where n and R₁₁ are as previously defined;

(h) —

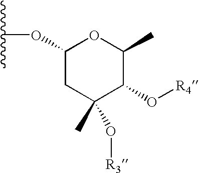

where R₃″ is hydrogen or methyl; R₄″ is hydrogen or Rₚ, where Rₚ is as previously defined; and

R₂′ is hydrogen or Rₚ, where Rₚ, is as previously defined.

In another alternate embodiment of the present invention are compounds of formula V:

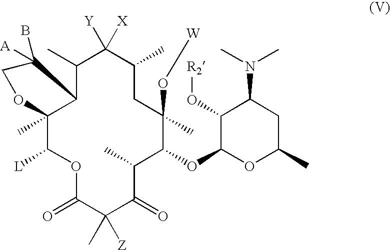

as well as pharmaceutically acceptable salts, esters and prodrugs thereof.

In formula V:

A is selected from:

(a) —OH;

(b) —OR₁, where R₁ is a hydroxy protecting group;

(c) —R₂, where R₂ is selected from:

1. aryl;

2. substituted aryl;

3. heteroaryl; and

4. substituted heteroaryl;

(d) OR₁, where R₁ is as previously defined;

(e) —R₂, where R₂ is selected from:

1. hydrogen;

2. halogen;

3. C₁-C₆ alkyl containing 0, 1, 2, or 3 heteroatoms selected from O, S and N, optionally substituted with one or more substituents selected from halogen, cyano, oxo, aryl, substituted aryl, heteroaryl and substituted heteroaryl;

4. C₂-C₆ alkenyl containing 0, 1, 2, or 3 heteroatoms selected from O, S and N, optionally substituted with one or more substituents selected from halogen, cyano, oxo, aryl, substituted aryl, heteroaryl and substituted heteroaryl; and

5. C₂-C₆ alkynyl containing 0, 1, 2, or 3 heteroatoms selected from O, S and N, optionally substituted with one or more substituents selected from halogen, cyano, oxo, aryl, substituted aryl, heteroaryl and substituted heteroaryl;

(f) —CR₂, where R₂ is previously defined;

(g) —S(O)ₙR ₁, where n=0, 1 or 2, and R₁₁ is selected from hydrogen, R₁ and R₂, where R₁ and R₂ are as previously defined;

(h) —OC(O)R₁₁, where R₁₁ is as previously defined;

(i) —C(O) R₁₁, where R₁₁ is as previously defined;

(j) —C(O)NHR₁₁, where R₁₁ is as previously defined;

(k) —OC(O)NHR₁₁, where R₁₁ is as previously defined;

(l) —NHC(O)R₁₁, where R₁₁ is as previously defined;

(m)—NHC(O)NHR₁₁, where R₁₁ is as previously defined;

(n) —NHS(O)ₙR₁₁, where n and R₁₁ are as previously defined;

(o) —NR₁₄R₁₅, where R₁₄ and R₁₅ are each independently R₁₁, where R₁₁ is as previously defined; and

(p) —NHR₃, where R₃ is an amino protecting group;

B is selected from:

(a) hydrogen;

(b) deuterium;

(c) —CN;

(d) —NO₂;

(e) halogen;

(f) —OH;

(g) —R₁, where R₁ is as previously defined;

(h) —R₂, where R₂ is as previously defined; and

(i) —ORₚ, where Rₚ is as previously defined;

provided that when B is halogen, —NO₂, —OH or ORₚ, A is R₁ or R₂;

or, alternatively, A and B taken together with the carbon atom to which they are attached are selected from:

(a) C═O;

(b) C(OR₂)₂, where R₂ is as previously defined;

(c) C(SR₂)₂, where R₂ is as previously defined;

(d) C(OR₁₂)(OR₁₃), where R₁₂ and R₁₃ taken together are —(CH₂)ₘ—, and where m is 2 or 3;

(e) C(SR₁₂)(SR₁₃), where R₁₂ and R₁₃ taken together are —(CH₂)ₘ, where m is as previously defined;

(f) C═CR₁₁R₁₄, where R₁₁ and R₁₄ are as previously defined;

(g) C═N—O—R₁₁, where R₁₁ is as previously defined;

(h) C═NNHR₁₁, where R₁₁ is as previously defined;

(i) C═NNHC(O)R₁₁, where R₁₁ is as previously defined;

(j) C═NN═CR₁₁R₁₄, where R₁₁ and R₁₄ are as previously defined;

(k) C═NNHC(O)NHR₁₁, where R₁₁ is as previously defined;

(l) C═NNHS(O)ₙR₁₁, where n and R₁₁ are as previously defined;

(m)C═NNHR₃, where R₃ is as previously defined; and

(n) C═NR₁₁, where R₁₁ is as previously defined;

one of X and Y is hydrogen and the other is selected from:

(a) hydrogen;

(b) deuterium;

(c) —OH;

(d) —ORₚ, where Rₚ is as previously defined; and

(e) —NR₄R₅, where R₄ and R₅ are each independently selected from:

1. hydrogen; and

2. C₁-C₁₂ alkyl, optionally substituted with one or more substituents selected from halogen, cyano, aryl, substituted aryl, heteroaryl and substituted heteroaryl; or

R₄ and R₅, taken together with the nitrogen atom to which they are attached form a 3-10 membered heteroalkyl ring containing 0-2 additional hetero atoms selected from the group consisting of O, S and N; or alternatively, X and Y taken together with the carbon atom to which they are attached are selected from:

(a) C═O;

(b) C═NR₁₁, where R₁₁ is as previously defined;

(c) C═NC(O)R₁₁, where R₁₁ is as previously defined;

(d) C═N—OR₆, where R₆ is selected from:

1. hydrogen;

2. —CH₂O(CH₂)₂OCH₃,

3. —CH₂O(CH₂O)ₙCH₃, where n is as previously defined;

4. —C₁-C₁₂ alkyl, optionally substituted with one or more substituents selected from halogen, cyano, aryl, substituted aryl, heteroaryl and substituted heteroaryl;

5. C₃-C₁₂ cycloalkyl;

6. C(O)—C₁-C₁₂ alkyl;

7. C(O)—C₃-C₁₂ cycloalkyl;

8. C(O)—R₁, where R₁ is as previously defined; and

9. —Si(Rₐ)(R_{b})(R_{c}), wherein Rₐ, R_{b} and R_{c} are each independently selected from C₁-C₁₂ alkyl, aryl and substituted aryl; and

(e) C═N—O—C(R₇)(R₈)—O—R₆, where R₆ is as previously defined, provided that R₆ is not C(O)—C₁-C₁₂ alkyl, C(O)—C₃-C₁₂ cycloalkyl, or C(O)—R₁; and R₇ and R₈ taken together with the carbon atom to which they are attached form a C₃-C₁₂ cycloalkyl group or each is independently selected from:

1. hydrogen; and

2. C₁-C₁₂ alkyl;

L is selected from:

(a) —CH(OH)CH₃;

(b) C₁-C₆ alkyl, optionally substituted with one or more substituents selected from halogen, cyano, aryl, substituted aryl, heteroaryl, and substituted heteroaryl;

(c) C₂-C₆ alkenyl, optionally substituted with one or more substituents selected from halogen, cyano, aryl, substituted aryl, heteroaryl, and substituted heteroaryl; and

(d) C₂-C₆ alkynyl, optionally substituted with one or more substituents selected from halogen, cyano, aryl, substituted aryl, heteroaryl, and substituted heteroaryl;

W is selected from:

(a) C₂-C₆ alkyl containing 0, 1, 2, or 3 heteroatoms selected from O, S and N, optionally substituted with one or more substituents selected from halogen, cyano, oxo, aryl, substituted aryl, heteroaryl, and substituted heteroaryl;

(b) C₂-C₆ alkenyl containing 0, 1, 2, or 3 heteroatoms selected from O, S and N, optionally substituted with one or more substituents selected from halogen, cyano, oxo, aryl, substituted aryl, heteroaryl, and substituted heteroaryl; and

(c) C₂-C₆ alkynyl containing 0, 1, 2, or 3 heteroatoms selected from O, S and N, optionally substituted with one or more substituents selected from halogen, cyano, oxo, aryl, substituted aryl, heteroaryl, and substituted heteroaryl;

Z is selected from:

(a) hydrogen;

(b) halogen; and

(c) C₁-C₆ alkyl, optionally substituted with one or more substituents selected from halogen, cyano, aryl, substituted aryl, heteroaryl and substituted heteroaryl; and

R₂′ is hydrogen or Rₚ, where Rₚ, is as previously defined.

In another aspect of the present invention there are disclosed pharmaceutical compositions comprising a therapeutically effective amount of a compound of the invention in combination with a pharmaceutically acceptable carrier, and treatment of antibacterial infections with such compositions. Suitable carriers and methods of formulation are also disclosed. The compounds and compositions of the present invention have antibacterial activity.

In a further aspect of the present invention there are provided processes for the preparation of 11, 12-cyclized erythromycin derivatives of formulae (I)-(V) via the synthetic methods delineated herein.

### DETAILED DESCRIPTION OF THE INVENTION

A first embodiment of the present invention includes compounds represented by formula I, as illustrated above, as well as the pharmaceutically acceptable salts, esters and prodrugs thereof.

In a preferred embodiment of the present invention are compounds of formula Ia:

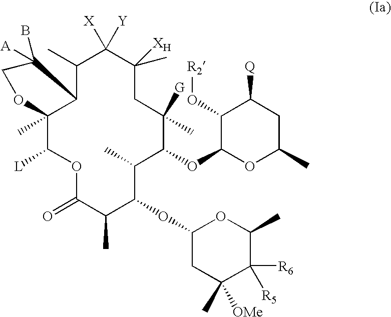

Representative compounds of the invention are those selected from:

### EXAMPLE 1

Compound of formula Ia: A and B taken together with the carbon atom to which they are attached are C═CH₂, L is CH₂CH₃, G is OCH₃, Q is N(CH₃)₂, X and Y taken together with the carbon atom to which they are attached are C═O, R₅ is Bz, R₆ is H, Rₑ is H, R_{f} is CH₃, X_{H} is H, and R₂ ′ is H;

### EXAMPLE 2

Compound of formula Ia: A and B taken together with the carbon atom to which they are attached are C═CH₂, L is CH₂CH₃, G is OCH₃, Q is N(CH₃)₂, X and Y taken together with the carbon atom to which they are attached are C═O, R₅ is OH, R₆ is H, X_{H} is H, and R₂′ is H;

### EXAMPLE 3

Compound of formula I: A and B taken together with the carbon atom to which they are attached are C═CH₂, L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H;

### EXAMPLE 4

Compound of formula Ia: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, R₅ is OBz, R₆ is H, X_{H} is H, and R₂′ is H;

### EXAMPLE 5

Compound of formula I: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H;

### EXAMPLE 6

Compound of formula Ia; A is H. B is —CH₂SC(O)CH₃, L is CH₂CH₃, G is OCH₃, Q is N(CH₃)₂, X and Y taken together with the carbon atom to which they are attached are C═O, R₅ is OBz, R₆ is H, X_{H} is H, and R₂′ is H;

### EXAMPLE 7

Compound of formula I: A is H, B is —CH₂SC(O)CH₃, L is CH₂CH₃, G is OCH₃, Q is N(CH₃)₂, X and Y taken together with the carbon atom to which they attached are C═O, U is OH, V is H, Rₑ is H, R_{f} is CH₃, X_{H} is H, and R₂′ is H;

### EXAMPLE 8

Compound of formula I: A is H, B is —CH₂SCH₂-(4-pyridyl), L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H;

### EXAMPLE 9

Compound of formula I: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H;

### EXAMPLE 10

Compound of formula I: A and B taken together with the carbon atom to which they are attached are C═CHSC(O)CH₃, L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H;

### EXAMPLE 11

Compound of formula Ia: A and B taken together with the carbon atom to which they are attached are C═CHSCH₂-phenyl, L is CH₂CH₃, G is OCH₃, Q is N(CH₃)₂, X and Y taken together with the carbon atom to which they are attached are C═O, R₅ is OBz, R₆ is H, X_{H} is H, and R₂′ is H;

### EXAMPLE 12

Compound of formula Ia: A and B taken together with the carbon atom to which they are attached are C═O, L is CH₂CH₃, G is OCH₃, Q is N(CH₃)₂, X and Y taken together with the carbon atom to which they are attached are C═O, R₅ is OBz, R₆ is H, X_{H} is H, and R₂′ is H;

### EXAMPLE 13

Compound of formula Ia: A is H and B is OH, L is CH₂CH₃, G is OCH₃, Q is N(CH₃)₂, X and Y taken together with the carbon atom to which they are attached are C═O, R₅ is OBz, R₆ is H, X_{H} is H, and R₂′ is H;

### EXAMPLE 14

Compound of formula I: A and B taken together with the carbon atom to which they are attached are C═CH₂, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 15

Compound of formula I: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 16

Compound of formula I: A and B taken together with the carbon atom to which they are attached are C═CHSO(CH₂)₂-phenyl, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 17

Compound of formula I: A is H, B is —CH₂SC(O)CH₃, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 18

Compound of formula I: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 19

Compound of formula I: A and B taken together with the carbon atom to which they are attached are C═CHSC(O)CH₃, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 20

Compound of formula I: A and B taken together with the carbon atom to which they are attached are C═CHSC(O)CH₃, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 21

Compound of formula I: A and B taken together with the carbon atom to which they are attached are C═CHSCH₂-phenyl, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 22

Compound of formula Ia: A and B taken together with the carbon atom to which they are attached are C═CH₂, L is CH₂CH₃, G is OCH₂CH═CH₂, Q is

N(CH₃)₂, X and Y taken together with the carbon atom to which they are attached are C═O, R₅ is OAc, R₆ is H, X_{H} is H, and R₂′ is H;

### EXAMPLE 23

Compound of formula Ia: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, R₅ is OH, R₆ is H, X_{H} is H, and R₂′ is H;

### EXAMPLE 24

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U is OH, V is H, Rₑ is H, R_{f} is CH₃, X_{H} is H, and R₂′ is H;

### EXAMPLE 25

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U is OH, V is H, Rₑ is H, R_{f} is CH₃, X_{H} is H, and R₂′ is H;

### EXAMPLE 26

Compound of formula Ia: A and B taken together with the carbon atom to which they are attached are C═CH₂, L is CH₂CH₃, G is OCH₂CH(O), Q is N(CH₃)₂, X and Y taken together with the carbon atom to which they are attached are C═O, R₂′ is H, X_{H} is H, R₅ is OH, and R₆ is H;

### EXAMPLE 27

Compound of formula Ia: A and B taken together with the carbon atom to which they are attached are C═CH₂, L is CH₂CH₃, G is OCH₂C≡CH, Q is N(CH₃)₂, X and Y taken together with the carbon atom to which they are attached are C═O, R₂′ is H, X_{H} is H, R₅ is OH, and R is H;

### EXAMPLE 28

Compound of formula Ia: A and B taken together with the carbon atom to which they are attached are C═CH₂, L is CH₂CH₃, G is OCH₂C≡C-(3-quinolyl), Q is N(CH₃)₂, X and Y taken together with the carbon atom to which they are attached are C═O, R₂′ is H, X_{H} is H, R₅ is OH, and R₆ is H;

### EXAMPLE 29

Compound of formula Ia: A and B taken together with the carbon atom to which they are attached are C═CH₂, L is CH₂CH₃, G is OCH₂CH₂NHCH₂-(4-chlorophenyl), Q is N(CH₃)₂, X and Y taken together with the carbon atom to which they are attached are C═O, R₂′ is H, X_{H} is H, R₅ is OH, and R₆ is H;

### EXAMPLE 30

Compound of formula Ia: A and B taken together with the carbon atom to which they are attached are C═CH₂, L is CH₂CH₃, G is OCH₂CH₂NCH₃CH₂-phenyl, Q is N(CH₃)₂, X and Y taken together with the carbon atom to which they are attached are C═O, R₂′ is H, X_{H} is H, R₅ is OH, and R₆ is H;

### EXAMPLE 31

Compound of formula Ia; A and B taken together with the carbon atom to which they are attached are C═CH₂, L is CH₂CH₃, G is OCH₂CH₂NCH₃CH₂-(2-pyridyl), Q is N(CH₃)₂, X and Y taken together with the carbon atom to which they are attached are C═O, R₂′ is H, X_{H} is H, R₅ is OH, and R₆ is H;

### EXAMPLE 32

Compound of formula Ia; A and B taken together with the carbon atom to which they are attached are C═CH₂, L is CH₂CH₃, G is OCH₂CH₂N(CH₃)CH₂-(3-pyridyl), Q is N(CH₃)₂, X and Y taken together with the carbon atom to which they are attached are C═O, R₂′ is H, X_{H} is H, R₅ is OH, and R₆ is H;

### EXAMPLE 33

Compound of formula Ia: A and B taken together with the carbon atom to which they are attached are C═CH₂, L is CH₂CH₃, G is OCH₂CH₂N(CH₃)CH₂-(3-quinolyl), Q is N(CH₃)₂, X and Y taken together with the carbon atom to which they are attached are C═O, R₂′ is H, X_{H} is H, R₅ is OH, and R₆ is H;

### EXAMPLE 34

Compound of formula Ia: A and B taken together with the carbon atom to which they are attached are C═CH₂, L is CH₂CH₃, G is OCH₂CH═CH-phenyl, Q is N(CH₃)₂, X and Y taken together with the carbon atom to which they are attached are C═O, R₂′ is H, X_{H} is H, R₅ is OH, and R₆ is H;

### EXAMPLE 35

Compound of formula Ia: A and B taken together with the carbon atom to which they are attached are C═CH₂, L is CH₂CH₃, G is OCH₂CH═CH-(2-pyridyl), Q is N(CH₃)₂, X and Y taken together with the carbon atom to which they are attached are C═O, R₂′ is H, X_{H} is H, R₅ is OH, and R₆ is H;

### EXAMPLE 36

Compound of formula Ia: A and B taken together with the carbon atom to which they are attached are C═CH₂, L is CH₂CH₃, G is OCH₂CH═CH-(3-pyridyl), Q is N(CH₃)₂, X and Y taken together with the carbon atom to which they are attached are C═O, R₂′ is H, X_{H} is H, R₅ is OH, and R₆ is H;

### EXAMPLE 37

Compound of formula Ia: A and B taken together with the carbon atom to which they are attached are C═CH₂, L is CH₂CH₃, G is OCH₂CH═CH-(3-(5-cyano)pyridyl), Q is N(CH₃)₂, X and Y taken together with the carbon atom to which they are attached are C═O, R₂′ is H, X_{H} is H, R₅ is OH, and R₆ is H;

### EXAMPLE 38

Compound of formula Ia: A and B taken together with the carbon atom to which they are attached are C═CH₂, L is CH₂CH₃, G is OCH₂CH═CH-(6-(aminocarbonyl)-3-quinolyl), Q is N(CH₃)₂, X and Y taken together with the carbon atom to which they are attached are C═O, R₂′ is H, X_{H} is H, R₅ is OH, and R₆ is H;

### EXAMPLE 39

Compound of formula Ia: A and B taken together with the carbon atom to which they are attached are C═CH₂, L is CH₂CH₃, G is OCH₂C≡C-phenyl, Q is N(CH₃)₂, X and Y taken together with the carbon atom to which they are attached are C═O, R₂′ is H, X_{H} is H, R₅ is OH, and R₆ is H;

### EXAMPLE 40

Compound of formula Ia: A and B taken together with the carbon atom to which they are attached are C═CH₂, L is CH₂CH₃, G is OCH₂C≡C-(2-pyridyl), Q is N(CH₃)₂, X and Y taken together with the carbon atom to which they are attached are C═O, R₂′ is H, X_{H} is H, R₅ is OH, and R₆ is H;

### EXAMPLE 41

Compound of formula Ia: A and B taken together with the carbon atom to is N(CH₃)₂, X and Y taken together with the carbon atom to which they are attached are C═O, R₂′ is H, X_{H} is H, R₅ is OH, and R₆ is H;

### EXAMPLE 42

Compound of formula Ia; A and B taken together with the carbon atom to which they are attached are C═CH₂, L is CH₂CH₃, G is OCH₂C≡C-(3-(5-cyano)pyridyl), Q is N(CH₃)₂, X and Y taken together with the carbon atom to which they are attached are C═O, R₂′ is H, X_{H} is H, R₅ is OH, and R₆ is H;

### EXAMPLE 43

Compound of formula Ia: A and B taken together with the carbon atom to which they are attached are C═CH₂, L is CH₂CH₃, G is OCH₂C≡C-(5-(2-pyridyl)-2-thienyl), Q is N(CH₃)₂, X and Y taken together with the carbon atom to which they are attached are C═O, R₂′ is H, X_{H} is H, R₅ is OH, and R₆ is H;

### EXAMPLE 44

Compound of formula Ia: A and B taken together with the carbon atom to which they are attached are C═CH₂, L is CH₂CH₃, G is OCH₂C≡C-(5-(3-pyridinyl)-2-pyrrolyl), Q is N(CH₃)₂, X and Y taken together with the carbon atom to which they are attached are C═O, R₂′ is H, X_{H} is H, R₅ is OH, and R₆ is H;

### EXAMPLE 45

Compound of formula Ia: A and B taken together with the carbon atom to which they are attached are C═CH₂, L is CH₂CH₃, G is OCH₂C≡C-(5-(2-pyrimidyl)-2-thienyl), Q is N(CH₃)₂, X and Y taken together with the carbon atom to which they are attached are C═(O, R₂′ is H, X_{H} is H, R₅ is OH, and R₆ is H;

### EXAMPLE 46

Compound of formula Ia: A and B taken together with the carbon atom to which they are attached are C═CH₂, L is CH₂CH₃, G is OCH₂C≡C-(5-(2-pyrazinyl)-2-pyrrolyl), Q is N(CH₃)₂, X and Y taken together with the carbon atom to which they are attached are C═O, R₂′ is H, X_{H} is H, R₅ is OH, and R₆ is H;

### EXAMPLE 47

Compound of formula Ia: A and B taken together with the carbon atom to which they are attached are C═CH₂, L is CH₂CH₃, G is OCH₂C≡C-(6-quinolyl), Q is N(CH₃)₂, X and Y taken together with the carbon atom to which they are attached are C═O, R₂′ is H, X_{H} is H, R₅ is OH, and R₆ is H;

### EXAMPLE 48

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 49

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 50

Compound of formula I: A and B taken together with the carbon atom to which they are attached are C═CH₂, L is CH₂CH₃, G is OCH₂CH═CH-(3-quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 51

Compound of formula I: A and B taken together with the carbon atom to which they are attached are C═CH₂, L is CH₂CH₃, G is OCH₂CH(O), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 52

Compound of formula I: A and B taken together with the carbon atom to which they are attached are C═CH₂, L is CH₂CH₃, G is OCH₂C═CH, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 53

Compound of formula I: A and B taken together with the carbon atom to which they are attached are C═CH₂, L is CH₂CH₃, G is OCH₂C≡C-(3-quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 54

Compound of formula I: A and B taken together with the carbon atom to which they are attached are C═CH₂, L is CH₂CH₃, G is OCH₂CH₂NHCH₂-(4-chlorophenyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 55

Compound of formula I: A and B taken together with the carbon atom to which they are attached are C═CH₂, L is CH₂CH₃, G is OCH₂C≡CH, Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 56

Compound of formula I: A and B taken together with the carbon atom to which they are attached are C═CH₂, L is CH₂CH₃, G is OCH₂C≡C-(3-quinolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 57

Compound of formula I: A and B taken together with the carbon atom to which they are attached are C═CH₂, L is CH₂CH₃, G is OCH₂CH₂NCH₃CH₂-phenyl, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 58

Compound of formula I: A and B taken together with the carbon atom to which they are attached are C═CH₂, L is CH₂CH₃, G is OCH₂CH₂NCH₃CH₂-(2-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 59

Compound of formula I: A and B taken together with the carbon atom to which they are attached are C═CH₂, L is CH₂CH₃, G is OCH₂CH₂NCH₃CH₂-(3-pyridyl), Q is N(CH₃)₂, X and Y taken together with the carbon atom to which they are attached are C═O, U and V taken together with the carbon atom to which hey are attached are C═O, Rₑ is H, R_{f} is CH₃, X_{H} is H, and R₂′ is H;

### EXAMPLE 60

Compound of formula I: A and B taken together with the carbon atom to which they are attached are C═CH₂, L is CH₂CH₃, G is OCH₂CH₂NCH₃CH₂-(3-quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 61

Compound of formula I: A and B taken together with the carbon atom to which they are attached are C═CH₂, L is CH₂CH₃, G is OCH₂CH₂NCH₃CH₂-(2-pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 62

Compound of formula I: A and B taken together with the carbon atom to which they are attached are C═CH₂, L is CH₂CH₃, G is OCH₂CH₂NCH₃CH₂-(3-quinolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 63

Compound of formula I: A and B taken together with the carbon atom to which they are attached are C═CH₂, L is CH₂CH₃, G is OCH₂CH═CH-phenyl, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 64

Compound of formula I: A and B taken together with the carbon atom to which they are attached are C═CH₂, L is CH₂CH₃, G is OCH₂CH═CH-(2-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 65

Compound of formula I: A and B taken together with the carbon atom to which they are attached are C═CH₂, L is CH₂CH₃, G is OCH₂CH═CH-(3-pyridyl), Q is N(CH₃)₂, e_X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 66

Compound of formula I: A and B taken together with the carbon atom to which they are attached are C═CH₂, L is CH₂CH₃, G is OCH₂CH═CH-(3-(5-cyano)pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 67

Compound of formula I: A and B taken together with the carbon atom to which they are attached are C═CH₂, L is CH₂CH₃, G is OCH₂CH═CH-(6-(aminocarbonyl)-3-quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 68

Compound of formula I: A and B taken together with the carbon atom to which they are attached are C═CH₂, L is CH₂CH₃, G is OCH₂CH═CH-(3-quinolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 69

Compound of formula I: A and B taken together with the carbon atom to which they are attached are C═CH₂, L is CH₂CH₃, G is OCH₂CH═CH-(3-pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 70

Compound of formula I: A and B taken together with the carbon atom to which they are attached are C═CH₂, L is CH₂CH₃, G is OCH₂C≡C-phenyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 71

Compound of formula I: A and B taken together with the carbon atom to which they are attached are C═CH₂, L is CH₂CH₃, G is OCH₂C≡C-(2-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 72

Compound of formula I: A and B taken together with the carbon atom to which they are attached are C═CH₂, L is CH₂CH₃, G is OCH₂C≡C-(3-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 73

Compound of formula I: A and B taken together with the carbon atom to which they are attached are C═CH₂, L is CH₂CH₃, G is OCH₂C≡C-(3-(5-cyano)pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 74

Compound of formula I: A and B taken together with the carbon atom to which they are attached are C═CH₂, L is CH₂CH₃, G is OCH₂C≡C-(5-(2-pyridyl)-2-thienyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 75

Compound of formula I: A and B taken together with the carbon atom to which they are attached are C═CH₂, L is CH₂CH₃, G is OCH₂C≡C-(5-(3-pyridinyl)-2-pyrrolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 76

Compound of formula I: A and B taken together with the carbon atom to which they are attached are C═CH₂, L is CH₂CH₃, G is OCH₂C≡C-(2-pyrimidyl)-2-thienyl), Q is N(CH₃)₂, and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 77

Compound of formula I: A and B taken together with the carbon atom to which they are attached are C═CH₂, L is CH₂CH₃, G is OCH₂C≡C-(5-(2-pyrazinyl)-2-pyrrolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 78

Compound of formula I: A and B taken together with the carbon atom to which they are attached are C═CH₂, L is CH₂CH₃, G is OCH₂C≡C-(6quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H;

### EXAMPLE 79

Compound of formula I: A and B taken together with the carbon atom to which they are attached are C═CH₂, L is CH₂CH₃, G is OCH₂C≡C-phenyl, Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 80

Compound of formula I: A and B taken together with the carbon atom to which they are attached are C═CH₂, L is CH₂CH₃, G is OCH₂C≡C-(2-pyridyl), Q is N(CH₃)₂, X and Y taken together with the carbon atom to which they are attached are C═O, U and V taken together with the carbon atom to which they are attached are C═O, Rₑ is F, R_{f} is CH₃, and R₂′ is H;

### EXAMPLE 81

Compound of formula I: A and B taken together with the carbon atom to which they are attached are C═CH₂, L is CH₂CH₃, G is OCH₂C≡C-(3-pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 82

Compound of formula I: A and B taken together with the carbon atom to which they are attached are C═CH₂, L is CH₂CH₃, G is OCH₂C≡C-(3-(5-cyano)pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 83

Compound of formula I: A and B taken together with the carbon atom to which they are attached are C═CH₂, L is CH₂CH₃, G is OCH₂C≡C-(5-(2-pyridyl)-2-thienyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 84

Compound of formula I: A and B taken together with the carbon atom to which they are attached are C═CH₂, L is CH₂CH₃, G is OCH₂C≡C-(5-(2-pyrimidyl)-2-thienyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H;

### EXAMPLE 85

Compound of formula I: A and B taken together with the carbon atom to which they are attached are C═CH₂, L is CH₂CH₃, G is OCH₂C≡C-(5-(2-pyridinyl)-2-pyrrolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H; or

### EXAMPLE 86

Compound of formula I: A and B taken together with the carbon atom to which they are attached are C═CH₂, L is CH₂CH₃, G is OCH₂C≡C-(5-(2-pyrazinyl)-2-pyrrolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

Definitions

Listed below are definitions of various terms used to describe this invention. These definitions apply to the terms as they are used throughout this specification and claims, unless otherwise limited in specific instances, either individually or as part of a larger group.

An “aliphatic group” is non-aromatic moiety that may contain any combination of carbon atoms, hydrogen atoms, halogen atoms, oxygen, nitrogen, sulfur or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

Suitable aliphatic or aromatic substituents include, but are not limited to, —F, —Cl, —Br, —I, —OH, protected hydroxy, aliphatic ethers, aromatic ethers, oxo, —NO₂, —CN, —C₁-C₁₂-alkyl optionally substituted with halogen (such as perhaloalkyls), C₂-C₁₂-alkenyl optionally substituted with halogen, —C₂-C₁₂-alkynyl optionally substituted with halogen, —NH₂, protected amino, —NH—C₁-C₁₂-alkyl, —NH—C₂-C₁₂-alkenyl, —NH—C₂-C₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkynyl, —CONH—C₃-C₁₂-cycloalkyl, —CONH-aryl, —CONH-heteroaryl, —CONH-heterocycloalkyl, —CO₂—C₁-C₁₂-alkyl, —CO₂—C₂-C₁₂-alkenyl, —CO₂—C₂-C₁₂-alkynyl, —CO₂—C₃-C₁₂-cycloalkyl, —CO₂-aryl, —CO₂-heteroaryl, —CO₂-heterocycloalkyl, —OCO₂—C₁-C₁₂-alkyl, —OCO₂—C₂-C₁₂-alkenyl, —OCO₂—C₂-C₁₂-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, NHC(O)NH—C₁-C₁₂-alkyl, —NHC(O)NH—C₂-C₁₂-alkenyl, —NHC(O)NH—C₂-C₁₂-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, NHC(S)NH—C₁-C₁₂-alkyl, —NHC(S)NH—C₂-C₁₂-alkenyl, —NHC(S)NH—C₂-C₁₂-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, NHC(NH)NH—C₁-C₁₂-alkyl, —NHC(NH)NH—C₂-C₁₂-alkenyl, —NHC(NH)NH—C₂-C₁₂-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, NHC(NH)—C₁-C₁₂-alkyl, —NHC(NH)—C₂-C₁₂-alkenyl, —NHC(NH)—C₂-C₁₂-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkynyl, —C(NH)NH—C₃-C₁₂-Cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkynyl, —S(O)—C₃-C₁₂-cycloalkyl, —S(O)-aryl, —S(O)-heteroaryl, —S(O)-heterocycloalkyl-SO₂NH₂, —SO₂NH—C₁-C₁₂-alkyl, —SO₂NH—C₂-C₁₂-alkenyl, —SO₂NH—C₂-C₁₂-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkynyl, —S—C₃-C₁₂-cycloalkyl, —S-aryl, —S-heteroaryl, -S-heterocycloalkyl, or methylthiomethyl. It is understood that the aryls, heteroaryls, alkyls and the like can be further substituted.

The terms “C₂-C₁₂ alkenyl” or “C₂-C₆ alkenyl,” as used herein, denote a monovalent group derived from a hydrocarbon moiety containing from two to twelve or two to six carbon atoms having at least one carbon-carbon double bond by the removal of a single hydrogen atom. Alkenyl groups include, but are not limited to, for example, ethenyl, propenyl, butenyl, 1-methyl-2-buten-1-yl, alkadienes and the like.

The term “substituted alkenyl,” as used herein, refers to a “C₂-C₁₂ alkenyl” or “C₂-C₆ alkenyl” group as previously defined, substituted by one, two, three or more aliphatic substituents.

The terms “C₂-C₁₂ alkynyl” or “C₂-C₆ alkynyl,” as used herein, denote a monovalent group derived from a hydrocarbon moiety containing from two to twelve or two to six carbon atoms having at least one carbon-carbon triple bond by the removal of a single hydrogen atom. Representative alkynyl groups include, but are not limited to, for example, ethynyl, 1-propynyl, 1-butynyl, and the like.

The term “substituted alkynyl,” as used herein, refers to a “C₂-C₁₂ alkynyl” or “C₂-C₆ alkynyl” group as previously defined, substituted by one, two, three or more aliphatic substituents.

The term “C₁-C₆ alkoxy,” as used herein, refers to a C₁-C₆ alkyl group, as previously defined, attached to the parent molecular moiety through an oxygen atom. Examples of C₁-C₆-alkoxy include, but are not limited to, methoxy, ethoxy, propoxy, isopropoxy, n-butoxy, sec-butoxy, tert-butoxy, n-pentoxy, neopentoxy and n-hexoxy.

The terms “halo” and “halogen,” as used herein, refer to an atom selected from fluorine, chlorine, bromine and iodine.

The terms “aryl” or “aromatic” as used herein, refer to a mono- or bicyclic carbocyclic ring system having one or two aromatic rings including, but not limited to, phenyl, naphthyl, tetrahydronaphthyl, indanyl, and the like.

The terms “substituted aryl” or “substituted aromatic,” as used herein, refer to an aryl or aromatic group substituted by one, two, three or more aromatic substituents.

The term “arylalkyl,” as used herein, refers to an aryl group attached to the parent compound via a C₁-C₃ alkyl or C₁-C₆ alkyl residue. Examples include, but are not limited to, benzyl, phenethyl and the like.

The term “substituted arylalkyl,” as used herein, refers to an arylalkyl group, as previously defined, substituted by one, two, three or more aromatic substituents.

The terms “heteroaryl” or “heteroaromatic,” as used herein, refer to a mono-, bi-, or tri-cyclic aromatic radical or ring having from five to ten ring atoms of which at least one ring atom is selected from S, O and N; zero, one or two ring atoms are additional heteroatoms independently selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like. The heteroaromatic ring may be bonded to the chemical structure through a carbon or hetero atom.

The terms “substituted heteroaryl” or “substituted heteroaromatic,” as used herein, refer to a heteroaryl or heteroaromatic group, substituted by one, two, three, or more aromatic substituents.

The term “C₃-C₁₂-cycloalkyl” or “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo [2.2.1] heptyl, and bicyclo [2.2.2] octyl.

The term “C₃-C₁₂-cycloalkyl” or “substituted alicyclic,” as used herein, refers to an alicyclic group substituted by one, two, three or more aliphatic substituents.

The term “heterocyclic” or “herterocycloalkyl,” as used herein, refers to a non-aromatic ring, comprising three or more ring atoms, or a bi- or tricyclic group fused system, where (i) each ring contains between one and three heteroatoms independently selected from oxygen, sulfur and nitrogen, (ii) each 5-membered ring has 0 to I double bonds and each 6-membered ring has 0 to 2 double bonds, (iii) the nitrogen and sulfur heteroatoms may optionally be oxidized, (iv) the nitrogen heteroatom may optionally be quaternized, (iv) any of the above rings may be fused to a benzene ring, and (v) the remaining ring atoms are carbon atoms which may be optionally oxo-substituted. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl.

The term “substituted heterocycloalkyl” or “substituted heterocyclic,” as used herein, refers to a heterocyclic group, as previously defined, substituted by one, two, three or more aliphatic substituents.

The term “heteroarylalkyl,” as used herein, to an heteroaryl group attached to the parent compound via a C₁-C₃ alkyl or C₁-C₆ alkyl residue. Examples include, but are not limited to, pyridinylmethyl, pyrimidinylethyl and the like.

The term “substituted heteroarylalkyl,” as used herein, refers to a heteroarylalkyl group, as previously defined, substituted by independent replacement of one, two, or three or more aromatic substituents.

The term “alkylamino” refers to a group having the structure —NH(C₁-C₁₂ alkyl).

The term “dialkylamino” refers to a group having the structure —N(C₁-C₁₂ alkyl) (C₁-C₁₂ alkyl)-, and cyclic amines. Examples of dialkylamino are, but not limited to, dimethylamino, diethylamino, methylethylamino, piperidino, morpholino and the like.

The term “alkoxycarbonyl” represents an ester group, i.e., an alkoxy group, attached to the parent molecular moiety through a carbonyl group such as methoxycarbonyl, ethoxycarbonyl, and the like.

The term “carboxaldehyde,” as used herein, refers to a group of formula —CHO.

The term “carboxy,” as used herein, refers to a group of formula —COOH.

The term “carboxamide,” as used herein, refers to a group of formula —C(O)NH(C₁-C₁₂ alkyl) or —C(O)N(C₁-C₁₂ alkyl) (C₁-C₁₂ alkyl), —C(O)NH₂, NHC(O)(C₁-C₁₂ alkyl), N(C₁-C₁₂ alkyl)C(O)(C₁-C₁₂ alkyl) and the like.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “protected hydroxy,” as used herein, refers to a hydroxy group protected with a hydroxy protecting group, as defined above, including benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. 1, in the Techniques of Chemistry Series, John Wiley & Sons, NY, 1986.

The term “protic solvent,” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protic solvents may be found in organic chemistry textbooks or in specialized monographs, for example: Organic Solvents Physical Properties and Methods of Purification, 4th ed., edited by John A. Riddick et al., Vol. 1, in the Techniques of Chemistry Series, John Wiley & Sons, NY, 1986.

The term “oxidizing agent(s),” as used herein, refers to reagents useful for oxidizing the 3-hydroxyl of the macrolide ring to the 3-carbonyl. Oxidizing agents suitable in the present process are either Swern oxidation reagents (dimethyl sulfoxide and an electrophilic compound selected from dicyclohexylcarbodiimide, acetic anhydride, trifluoroacetic anhydride, oxalyl chloride, or sulfur trioxide), Dess Martin oxidation reagents, or Corey-Kim oxidation reagents. A preferred method of oxidation is the use of the Corey-Kim oxidation reagents N-chlorosuccinimide-dimethyl sulfide complex.

The term “palladium catalyst,” as used herein, refers to optionally supported palladium(0) such as palladium metal, palladium on carbon, palladium on acidic, basic, or neutral alumina, and the like; palladium(0) complexes such as tetrakis(triphenylphosphine)palladium(0) tris(dibenzylideneacetone)dipalladium(0); palladium(II) salts such as palladium acetate or palladium chloride; and palladium(II) complexes such as allylpalladium(II) chloride dimer, (1,1′-bis(diphenylphosphino)ferrocene)-dichloropalladium(II), bis(acetato)bis(triphenylphosphine)palladium(II), and bis(acetonitrile)dichloropalladium(II).

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present invention. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995).

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)-for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluensulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds formed by the process of the -present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The term “subject” as used herein refers to an animal. Preferably the animal is a mammal. More preferably the mammal is a human. A subject also refers to, for example, dogs, cats, horses, cows, pigs, guinea pigs, fish, birds and the like.

This invention also encompasses pharmaceutical compositions containing, and methods of treating bacterial infections in a subject through administering, pharmaceutically acceptable prodrugs of compounds produced by the process of the present invention. For example, compounds having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula I. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beeta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Suitable concentrations of reactants are 0.01M to 10M, typically 0.1M to 1M. Suitable temperatures include −10° C. to 250° C., typically −78° C. to 150° C., more typically −78° C. to 100° C., still more typically 0° C. to 100° C. Reaction vessels are preferably made of any material which does not substantial interfere with the reaction. Examples include glass, plastic, and metal. The pressure of the reaction can advantageously be operated at atmospheric pressure. The atmospheres includes, for example, air, for oxygen and water insensitive reactions, or nitrogen or argon, for oxygen or water sensitive reactions.

An “effective amount,” as used herein, refers to an amount of a compound which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

As used herein, unless otherwise indicated, the term “bacterial infection(s)” or “protozoa infections”; includes, but is not limited to, bacterial infections and protozoa infections that occur in mammals, fish and birds as well as disorders related to bacterial infections and protozoa infections that may be treated or prevented by administering antibiotics such as the compounds of the present invention. Such bacterial infections and protozoa infections and disorders related to such infections include, but are not limited to, the following: pneumonia, otitis media, sinusitis, bronchitis, tonsillitis, and mastoiditis related to infection by Streptococcus pneumoniae, Haemophilus influenzae, Moraxella catarrhalis, Staphylococcus aureus, or Peptostreptococcus spp. Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S. and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp. Odontogenic infection related to infection by viridans streptococci; persistent cough related to infection by Bordetella pertussis; gas gangrene related to infection by Clostridium perfringens or Bacteroides spp.; Skin infection by S. aureus, Propionibacterium acne; atherosclerosis related to infection by Helicobacter pylori or Chlamydia pneumoniae; or the like.

Bacterial infections and protozoa infections and disorders related to such infections that may be treated or prevented in animals include, but are not limited to, the following: bovine respiratory disease related to infection by P. haemolytica., P. multocida, Mycoplasma bovis, or Bordetella spp.; cow enteric disease related to infection by E. coli or protozoa (i.e., coccidia, cryptosporidia, etc.), dairy cow mastitis related to infection by S. aureus, S. uberis, S. agalactiae, S. dysgalactiae, Klebsiella spp., Corynebacterium, or Enterococcus spp.; swine respiratory disease related to infection by A. pleuropneumoniae., P. multocida, or Mycoplasma spp.; swine enteric disease related to infection by E. coli, Lawsonia intracellularis, Salmonella spp., or Serpulina hyodyisinteriae; cow footrot related to infection by Fusobacterium spp.; cow metritis related to infection by E. coli; cow hairy warts related to Infection by Fusobacterium necrophorum or Bacteroides nodosus; cow pink-eye related to infection by Moraxella bovis, cow premature abortion related to infection by protozoa (i.e. neosporium); urinary tract infection in dogs and cats related to infection by E. coli; skin and soft tissue infections in dogs and cats related to infection by S. epidermidis, S. intermedius, coagulase neg. Staphylococcus or P. multocida; and dental or mouth infections in dogs and oats related to infection by Alcaligenes spp., Bacteroides spp., Clostridium spp., Enterobacter spp., Eubacterium spp., Peptostreptococcus spp., Porphfyromonas spp., Campylobacter spp., Actinomyces spp., Erysipelothrix spp., Rhodococcus spp., Trypanosoma spp., Plas,odium spp., Babesia spp., Toxoplasma spp., Pneumocystis spp., Leishmania spp., and Trichomonas spp., or Prevotella spp. Other bacterial infections and protozoa infections and disorders related to such infections that may be treated or prevented in accord with the method of the present invention are referred to in J. P. Sanford at al.,“The Sanford Guide To Antimicrobial Therapy,” 26^{th} Edition, (Antimicrobial Therapy, Inc., 1996).

Antibacterial Activity

Susceptibility tests can be used to quantitatively measure the in vitro activity of an antimicrobial agent against a given bacterial isolate. Compounds were tested for in vitro antibacterial activity by a micro-dilution method. Minimal Inhibitory Concentration (MIC) was determined in 96 well microtiter plates utilizing the appropriate Mueller Hinton Broth medium (CAMHB) for the observed bacterial isolates. Antimicrobial agents were serially diluted (2-fold) in DMSO to produce a concentration range from about 64 μg/ml to about 0.03 μg/ml. The diluted compounds (2 μl/well) were then transferred into sterile, uninoculated CAMHB (0.2 mL) by use of a 96 fixed tip-pipetting station. The inoculum for each bacterial strain was standardized to 5×10⁵ CFU/mL by optical comparison to a 0.5 McFarland turbidity standard. The plates were inoculated with 10 μl/well of adjusted bacterial inoculum. The 96 well plates were covered and incubated at 35±2° C. for 24 hours in ambient air environment. Following incubation, plate wells were visually examined by Optical Density measurement for the presence of growth (turbidity). The lowest concentration of an antimicrobial agent at which no visible growth occurs was defined as the MIC. The compounds of the invention generally demonstrated an MIC in the range from about 64 μg/ml to about 0.03 μg/ml.

All in vitro testing follows the guidelines described in the Approved Standards M7-A4 protocol, published by the National Committee for Clinical Laboratory Standards (NCCLS).

Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminun hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and I) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, bacterial infections are treated or prevented in a patient such as a human or other animals by administering to the patient a therapeutically effective amount of a compound of the invention, in such amounts and for such time as is necessary to achieve the desired result.

By a “therapeutically effective amount” of a compound of the invention is meant a sufficient amount of the compound to treat bacterial infections, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between I mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The pharmaceutical compositions of this invention can be administered orally to fish by blending said pharmaceutical compositions into fish feed or said pharmaceutical compositions may be dissolved in water in which infected fish are placed, a method commonly referred to as a medicated bath. The dosage for the treatment of fish differs depending upon the purpose of administration (prevention or cure of disease) and type of administration, size and extent of infection of the fish to be treated. Generally, a dosage of 5-1000 mg, preferably 20-100 mg, per kg of body weight of fish may be administered per day, either at one time or divided into several times. It will be recognized that the above-specified dosage is only a general range which may be reduced or increased depending upon the age, body weight, condition of disease, etc. of the fish.

Unless otherwise defined, all technical and scientific terms used herein are accorded the meaning commonly known to one with ordinary skill in the art. All publications, patents, published patent applications, and other references mentioned herein are hereby incorporated by reference in their entirety

Abbreviations

Abbreviations which may be used in the descriptions of the schemes and the examples that follow are: Ac for acetyl; AIBN for 2,2′-azobisisobutyronitrile; Bn for benzyl; Boc for t-butoxycarbonyl; Bu₃SnH for tributyltin hydride; Bz for benzoyl; CDI for carbonyldiimidazole; dba for dibenzylidene acetone; dppb for diphenylphosphino butane; DBU for 1,8-diazabicyclo [5.4.0]undec-7-ene; DCC for 1,3-dicyclohexylcarbodiimide; DEAD for diethylazodicarboxylate; DIBAL-H for diisopropyl aluminum hydride; DIC for 1,3-diisopropylcarbodiimide; DIEA for diisopropylethylamine; DMAP for dimethylaminopyridine; DMF for dimethyl formamide; DMSO for dimethylsulfoxide; DPPA for diphenylphosphoryl azide; LAH for lithium aluminum hydride; EtOAc for ethyl acetate; KHMDS for potassium bis (trimethylsilyl) amide; LDA for lithium diisopropyl amide; MeOH for methanol; Me₂S for dimethyl sulfide; MOM for methoxymethyl; OMs for mesylate; OTos for tosylate; NaN(TMS)₂ for sodium bis(trimethylsilyl)amide; NCS for N-chlorosuccinimide; NMMO for 4-methylmorpholine N-oxide; PCC for pyridinium chlorochromate; PDC for pyridinium dichromate; Ph for phenyl; POPd for dihydrogen dichlorobis(di-tert-butylphosphino)palladium(II); TEA for triethylamine; THF for tetrahydrofuran; TPP or PPh₃ for triphenylphosphine; TBS for tert-butyl dimethylsilyl; and TMS for trimethylsilyl.

Synthetic Methods

The compounds and processes of the present invention will be better understood in connection with the following synthetic schemes which are illustrative of the methods by which the compounds of the invention may be prepared.

A preferred intermediate of the present invention are compounds of formula Ib:

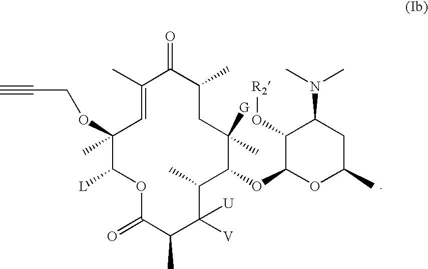

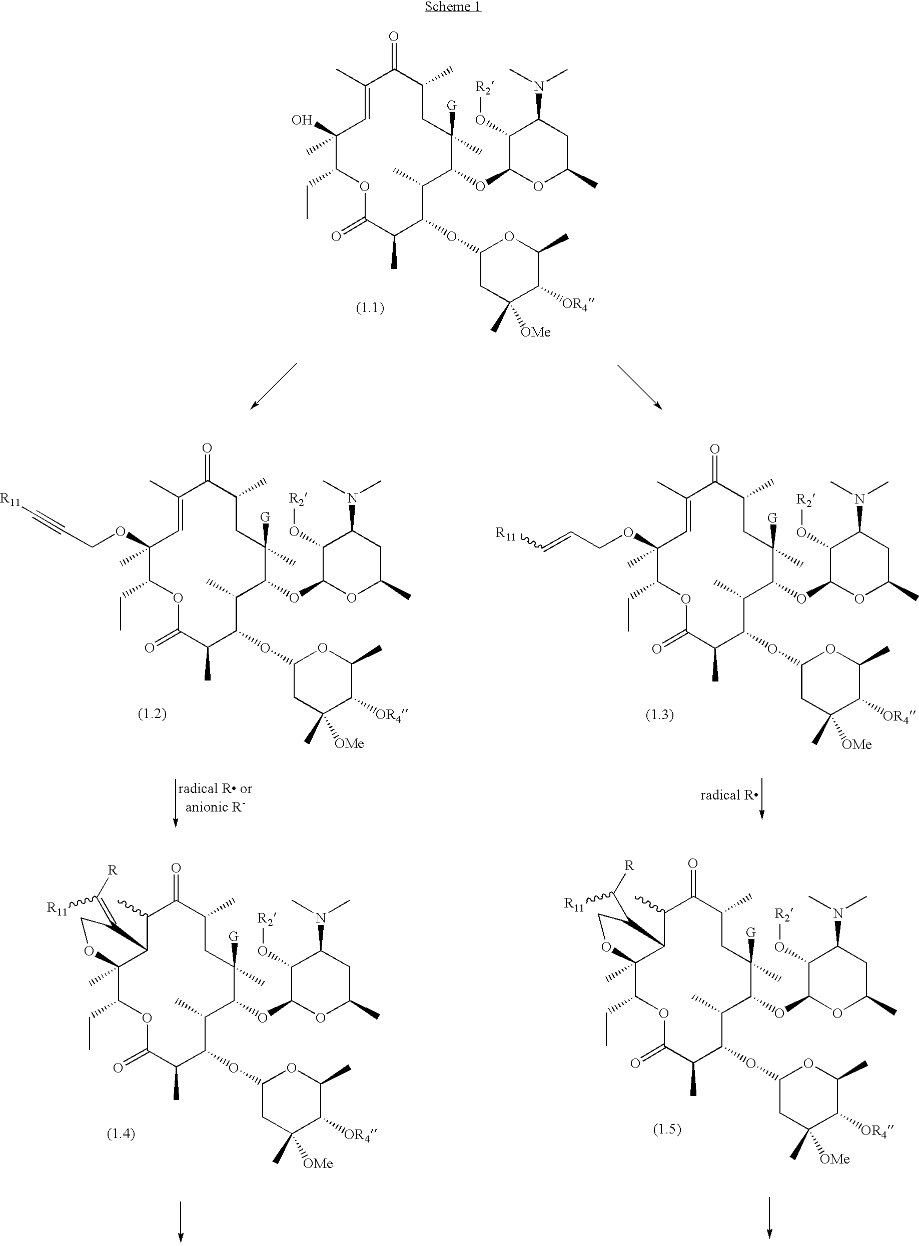

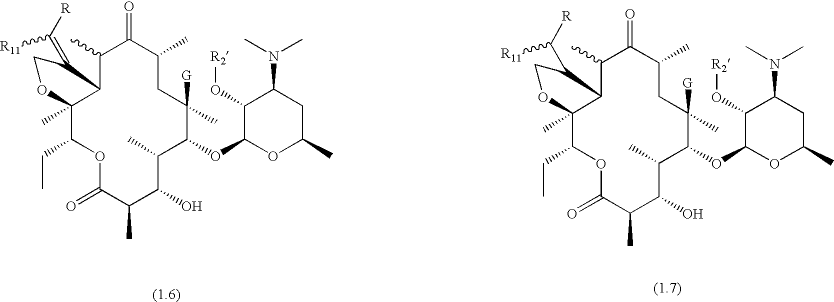

A process of the invention for the preparation of compounds of formula I, as illustrated in Scheme l, involves preparing compounds of formula (1.4) and (1.5) by a tandem radical or anionic addition and cyclization of compounds of formula (1.2) or (1.3).

Intermediates (1.2) and (1.3) can be prepared by alkylation of the readily available compounds of formula (1.1) which can be prepared according to the process described by Baker et al. J. Org. Chem. 1988, 53, 2340-2345; Elliott et al. J. Med. Chem. 1988, 41, 1651-1659; Ma et al. J. Med. Chem. 2001, 44, 4137-4156, and Or et al. U.S. Pat. No. 6,075,011-B1. Typical alkylating conditions include treating compounds of formula (1.1) with a suitable alkylating agent, such as propargyl halide, allyl halide, allyl mesylate or the like, in the presence of a base such as K₂CO₃, NaOH, NaH, LDA or the like, optionally with a phase transfer catalyst such as tetrabutylammonium iodide, 18-crown-6 or the like, in THF, toluene, methylene chloride, DMF, DMSO, water or the like, or combinations thereof, at from about −50° C. to about 100° C. for 1 hour to 24 hours to provide compounds of formula (1.2) and (1.3). Alternatively, compounds of formula (1.3) can be obtained by reaction of a suitable alkylating agent such as tert-butyl allyl carbonate, tert-butyl 2-butenyl carbonate, allyl acetate, allyl benzoate or the like, in the presence of a palladium catalyst, such as palladium(II) acetate, tetrakis(triphenylphosphine)palladium(0), tris(dibenzylideneacetone)dipalladium(0), tetra(dibenzylideneacetone)dipalladium(0), palladium on carbon or the like, and a suitable phosphine ligand, such as triphenylphosphine, bis(diphenylphosphino)methane, 1,2-bis(diphenylphosphino)ethane, 1,3-bis(diphenylphosphino)propane, 1,4-bis(diphenylphosphino)butane, tri-o-tolyl-phosphine, or the like, in an aprotic solvent, such as tetrahydrofuran, N,N-dimethylformamide, dimethyl sulfoxide, N-methyl-2-pyrrolidinone, hexamethylphosphoric triamide, 1,2-dimethoxyethane, methyl-tert-butyl ether, heptane, acetonitrile, acetonitrile and ethyl acetate or the like, at from 40° C. to about 150° C. for 0.5 hour to about 48 hours.

In accordance with Scheme 1, compounds of formula (1.4) and (1.5) of the present invention can be prepared by methods which are well known in the art involving a tandem radical addition and cyclization of intermediates (1.2) and (1.3) with a suitable radical species (R⁺) which can be generated from a radical precursor and an initiator. The radical R⁺ can be centered as, but not limited to, carbon, silicon, tin, oxygen, sulfur, nitrogen, halogen with non-, mono-, di- or tri-substitution depending on the nature of the radical centered atom. A typical radical of this process is selected from, but not limited to, a group consisting of PhCH₂⁺, Et₃Si, (n-Bu)₃Sn⁺, tert-BuO⁺, AcS⁺, PhCH₂CH₂S⁺ and Br⁺. A typical radical precursor for this process is selected from, but not limited to, C₁-C₁₂ alkyl halide, C₂-C₆ alkenyl halide, C₂-C₆ alkynyl halide, C₂-C₆ alkenyl tri(C₁-C₁₂ alkyl)stannane, tri(C₁-C₁₂ alkyl)stannane, hexamethyldistannane, trichlorosilane, triphenylsilane, tert-butyl hydrogen peroxide, thiolacetic acid, phenyl disulfide, N-bromosuccinamide and bromine. A typical radical initiator of this process can be selected from, but not limited to, a group consisting of AIBN, tert-butyl peroxide, benzoyl peroxide. The preferred radical reaction conditions of the present invention includes reacting the compounds of formula (1.2) or (1.3) with a radical generated from a group consisting of, but not limited to, halide, stannane, distannane, silane, mercaptan or disulfide, in the presence of AIBN, optionally in the presence of a reducing agent such as tributylstannane, diphenylsilane, sodium borohydride, magnesium, lithium aluminum hydride or the like, at 40° C. to 150° C. for a period of from 1 hour to 10 days, in an aprotic solvents, such as tetrahydrofuran, N,N-dimethylformamide, dimethyl sulfoxide, N-methyl-2-pyrrolidinone, 1,2-dimethoxyethane, methyl-tert-butyl ether, cyclohexane, heptane, acetonitrile, benzene, toluene and ethyl acetate or the like.

Alternatively compounds of formula (1.4) of the present invention may be prepared by a tandem anionic addition and cyclization of intermediates (1.2) with a suitable anionic species (R⁻) which can be generated from an organometallic precursor. Typically a compound of formula (1.2) is reacted with an organometallic reagents, such as allylmagnesium chloride, methylmagnesium iodide, phenyllithium, triethylaluminum, triethoxysilane, or the like, in the presence of 0-100% molar percent (relative to compound 1.2) of a transitional metal or its salt or its complex such as palladium, iridium, chromium(III) chloride, cerium(III) chloride, palladium(II) acetate, platinum(II) chloride, chloroplatinic acid, nonacabonyliron(0), titanocene (IV) dichloride, bis(1,5-cyclooctadiene)nickel(0), tetrakis(triphenylphosphine)palladium(0) or the like, at −78° C. to 100° C. for a period of from 0.5 to 48 hours, in an aprotic solvents, such as tetrahydrofuran, dimethyl sulfoxide, N-methyl-2-pyrrolidinone, 1,2-dimethoxyethane, methyl-tert-butyl ether, cyclohexane, heptane, acetonitrile, benzene and toluene or the like.

Another process of the invention involves the removal of the cladinose moiety of the compounds of formula I. The cladinose moiety of the macrolide compounds of formulae (1.4) and (1.5) can be removed to give compounds of formulae (1.6) and (1.7) in Scheme 1 by a dilute acid, such as hydrochloric acid, sulfuric acid, perchloric acid, nitric acid, chloroacetic acid, dichloroacetic acid, trifluoroacetic acid and p-toluenesulfonic acid or the like, in a suitable solvent, such as methanol, ethanol, isopropanol, butanol, water or the like, or the mixtures thereof, at 0° C. to about 80° C. for 0.5 hour to 24 hours.

When R₂″ is an acyl protecting group, it can be removed upon treatment with methanol at from room temperature to 60° C. When R₂″ is a silyl protecting group, the deprotection can be also effected by an acid, such as dilute hydrochloric acid, sulfuric acid, perchloric acid, nitric acid, chloroacetic acid, dichloroacetic acid, trifluoroacetic acid and p-toluenesulfonic acid or the like, or a fluoride, such as tetrabutylammonium fluoride, pyridinium fluoride, ammonium fluoride, hydrofluoric acid or the like, at from 0C to 50° C. for 0.5 to 24 hours.

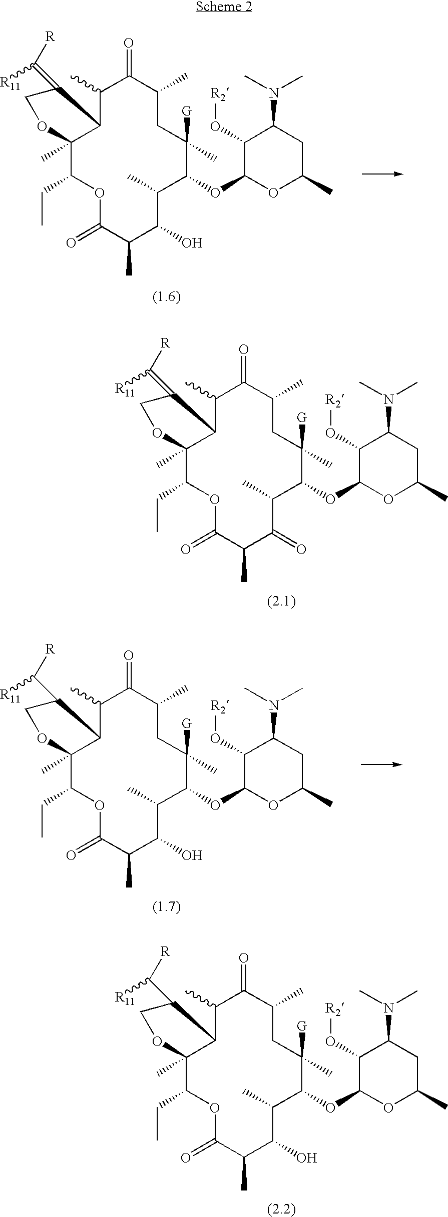

Compounds according to the invention (2.1) and (2.2) may be prepared by oxidation of the secondary alcohol using Dess-Martin periodinane as the oxidant. The reaction is typically run in an aprotic solvent at 0° to 25° C. for 0.5 to 12 hours.

Alternatively the oxidation can be accomplished using pyridinium chlorochromate, sulfur trioxide pyridine complex in dimethyl sulfoxide, tetra-n-propyl ammonium perruthenate and N-methyl morpholine N-oxide, Swern oxidation or the like. A more thorough discussion of the oxidation of secondary alcohols can be found in M. B. Smith and J. March “Advanced Organic Chemistry” 5^{th} ed., Wiley & Son, Inc, 2001, which is hereby incorporated by reference herein.

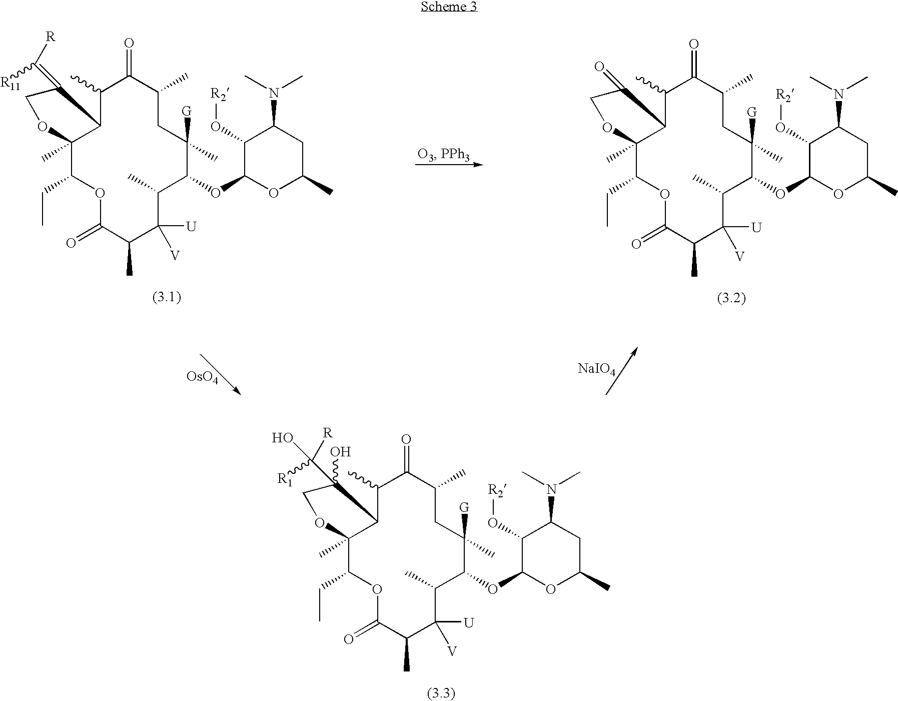

Conversion of the alkene of formula (3.1) into the ketone (3.2) can be accomplished by exposure of the alkene to ozone followed by decomposition of the ozonide intermediate with an appropriate reducing agent, as outlined in Scheme 3. The reaction is typically carried out in a solvent such as, for example, methanol, ethanol, ethyl acetate, glacial acetic acid, chloroform, methylene chloride or hexanes, or mixtures thereof, at from −78° C. to −20° C. Representative reducing agents include, for example, triphenylphosphine, trimethyl phosphate, thiourea, and dimethyl sulfide or the like. A more thorough discussion of ozonolysis and the conditions therefore can be found in M. B. Smith and J. March “Advanced Organic Chemistry” 5^{th} ed., Wiley & Son, Inc, 2001.

An alternative method for the preparation of the ketones (3.2) involves dihydroxylation of the alkene followed by diol cleavage. The glycol (3.3) is prepared by reacting the alkene (3.1), either with stoichiomettic amounts of osmium tetraoxide, or with catalytic amounts of osmium tetraoxide if an oxidant such as hydrogen peroxide, tert-butyl hydroperoxide, or N-methylmorpholine-N-oxide is present, in a variety of solvents such as 1,4-dioxane, tetrahydrofuran, tert-butanol, acetone, diethyl ether, water or the like, or the mixture thereof, preferably at from 0° C. to 50° C.

The glycol (3.3) can be cleaved by a variety of reagents including, but not limited to, periodic acid, lead tetraacetate, manganese dioxide, potassium permanganate, sodium metaperiodate, and N-iodosuccinamide in a variety of solvents such as 1,4-dioxane, tetrahydrofuran, tert-butanol, acetone, ethanol, methanol, water or the like, or the mixture thereof, at from 0° C. to 50° C.

The synthesis of the ketone (3.2) can also be realized in one-pot by reacting the alkene (3.1) with either stoichiometric amounts or catalytic amounts of osmium tetraoxide and a glycol cleavage reagent, such as, for example, periodic acid, lead tetraacetate, manganese dioxide, potassium permanganate, sodium metaperiodate, and N-iodosuccinamide or the like, in a solvent such as 1,4-dioxane, tetrahydrofuran, tert-butanol, acetone, ethanol, methanol, water or the like, or mixtures thereof, at from 0° C. to 50° C.

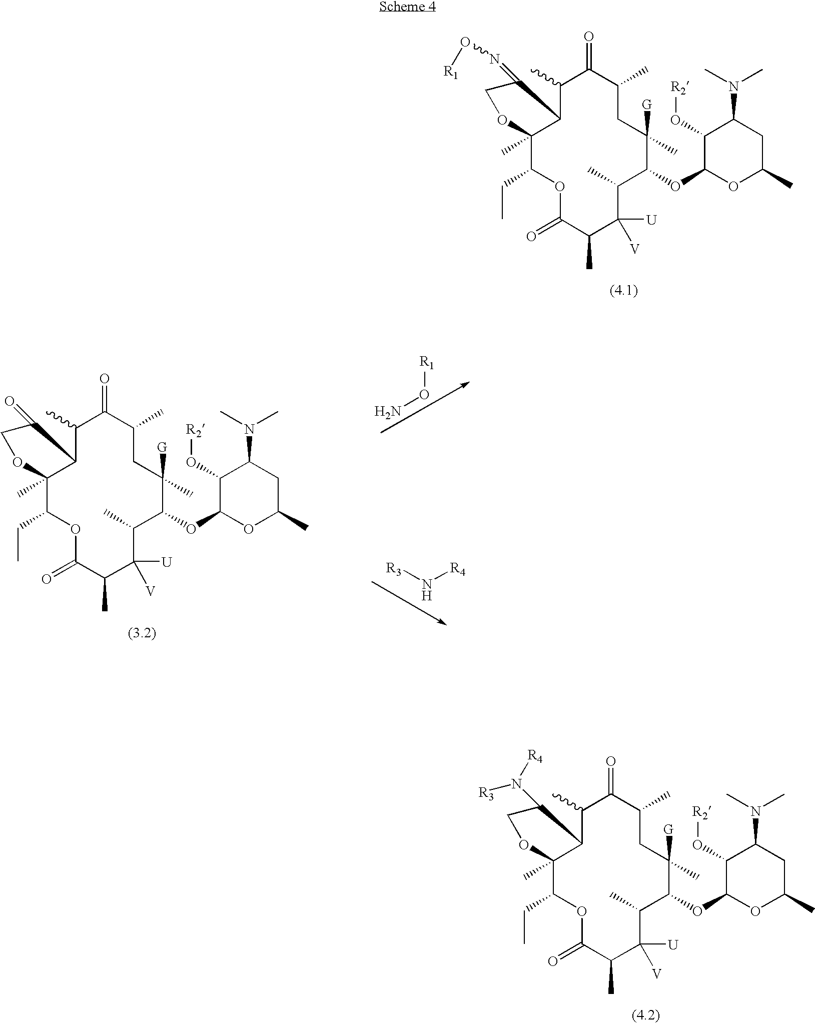

Compounds of formula (3.2) represent useful intermediates which can be further functionalized in a variety of ways. Scheme 4 details procedures for the conversion of the ketone (3.2) into an oxime of formula (4.1) or an amine of formula (4.2). The formation of oxime (4.1) can be accomplished under either acidic or basic conditions in a variety of solvents such as, for example, methanol, ethanol, water, tetrahydrofuran, 1,2-dimethoxyethane, ethyl acetate, or mixtures thereof, at from 0° C. to 70° C. over a period of 10 minutes to 12 hours. Representative acids include, but are not limited to, hydrochloric acid, phosphoric acid, sulfuric acid, p-toluenesulfonic acid, acetic acid and pyridinium p-toluenesulfonate. Bases which are useful are, for example, triethylamine, pyridine, diisopropylethyl amine, 2,6-lutidine, imidazole and potassium carbonate, and the like. The formation of amines (4.2) can be accomplished by reacting a ketone (3.2) with a primary or secondary amine and a suitable reducing agent such as, for example, hydrogen, sodium borohydride, sodium cyanoborohydride, LAH, zinc, DIBAL-H, triethylsilane, ammonium formate and the like, optionally in the presence of a catalyst such as Raney Ni, palladium on carbon, platinum dioxide, tetrakis(triphenylphosphine)palladium and the like in a suitable solvent such as methanol, acetonitrile, water, tetrahydrofuran, 1,2-dimethoxyethane, ethyl acetate, acetic acid, trifluoroacetic acid, hydrochloric acid or the like, or mixtures thereof, at a pH between 3 and 5 over a period of 5 minutes to 24 hours.

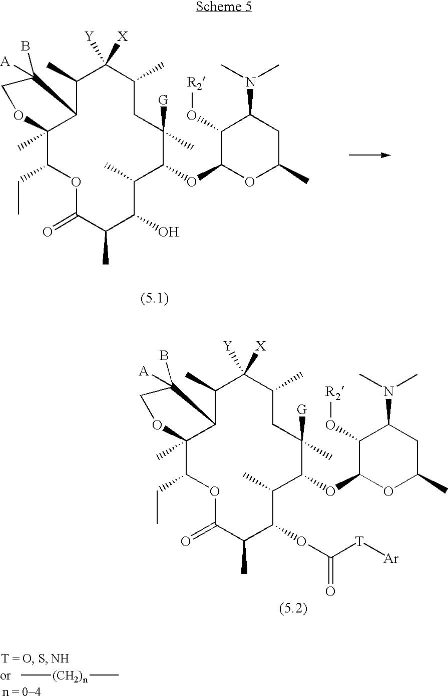

Scheme 5 illustrates a procedure for the acylation of the C-3 hydroxyl of compounds of formula (5.1). The hydroxyl group is reacted with an acylating agent such as, but not limited to, acid chlorides, acid anhydrides, and chloroformates in the presence of a base such as pyridine, triethylamine, diisopropyl ethylamine, N-methyl morpholine, N-methyl pyrrolidine, 2,6-lutidine, 1,8-diazabicyclo[5.4.0]undec-7-ene, and DMAP or the like, in an aprotic solvent. For a more extensive discourse on acylating conditions see for example, T. W. Greene and P. G. M. Wuts in “Protective Groups in Organic Synthesis” 3^{rd} ed., John Wiley & Son, Inc, 1999, referred to above herein.

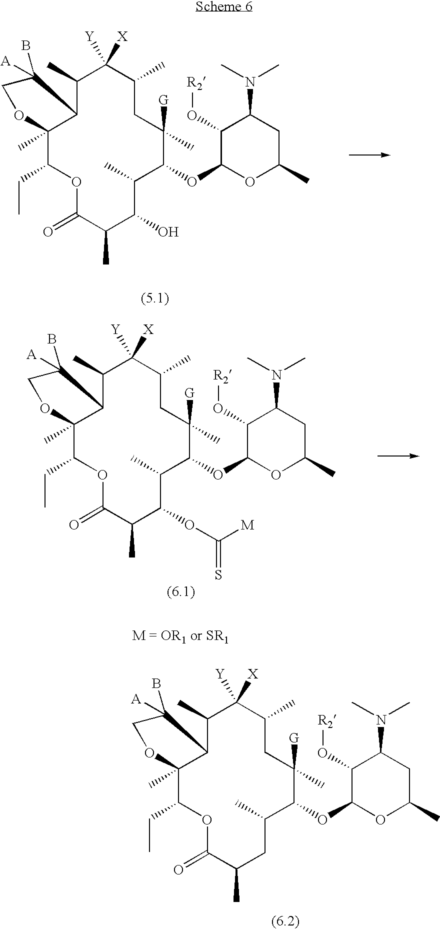

Another process of the invention, as illustrated in Scheme 6, involves the C-3 deoxygenation of the macrolide (5.1) which can be accomplished via a two step procedure shown therein through a xanthate or thiocarbonate of formula (6.1). In the first step, the xanthate is formed by the reaction of alkoxide of alcohol (5.1) with an appropriate thiocarbonyl reagent, such as carbondisulfide followed by methyliodide, or a dithiocarbonyl imidazole; whereas the thiocarbonate can be prepared by the reaction of the alkoxide with either thiocarbonyldiimidazole followed by methanol, ethanol or the like, or a thiochloroformate. One skilled in the art will appreciate that other reagents and conditions exist to perform these transformations and that the examples above are for illustrative purposes only and do not limit the scope of this invention. These reactions are typically run in a polar aprotic solvent, such as tetrahydrofuran, acetonitrile, N,N-dimethylformamide or the like.

In the second step of Scheme 6, the thiocarbonate or xanthate is reduced to give the alkane of formula (6.2). Most typically this is done under radical conditions using, for example, a silane or stannane such as (TMS)₃SiH, Ph₂SiH₂, BU₃SnH, Ph₃SnH or the like, and a radical initiator such as AIBN, tert-butyl hydrogen peroxide or the like in an aprotic solvent, such as tetrahydrofuran, N,N-dimethylformamide, dimethyl sulfoxide, N-methyl-2-pyrrolidinone, 1,2-dimethoxyethane, methyl tert-butyl ether, cyclohexane, heptane, acetonitrile, benzene, toluene and ethyl acetate or the like, at 0° C. to 150° C. for a period of from 1 hour to 10 days.

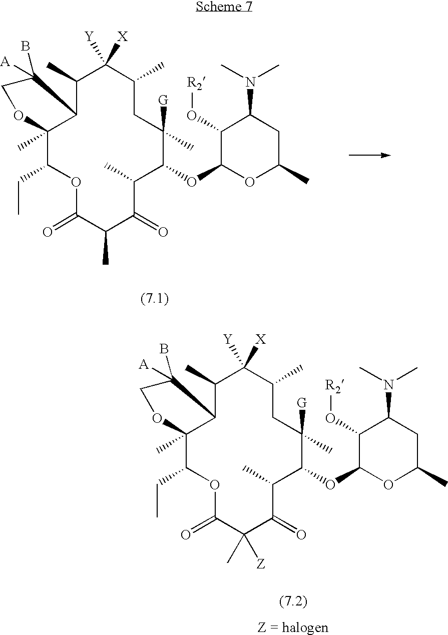

Scheme 7 illustrates the procedure by which compounds of formula (7.1) may be converted to compounds of formula (7.2) by treatment with a halogenating reagent in a suitable solvent such as dimethylformamide, dimethyl sulfoxide, pyrrolidinone and the like. By the process disclosed in U.S. Pat. No. 6,124,269 and International Patent WO 00/62783, which are hereby incorporated by reference herein in their entirety. This reagent acts to replace a hydrogen atom with a halogen atom at the C-2 position of the ketolide. Various halogenating reagents may be suitable for this procedure.

Fluorinating reagents include, but are not limited to, N-fluorobenzenesulfonimide in the presence of base, 10% F₂ in formic acid, 3,5-dichloro-1-fluoropyridinium tetrafluoroborate, 3,5-dichloro-1-fluoropyridinium triflate, (CF₃SO₂)₂NF, N-fluoro-N-methyl-p-toluenesulfonamide in the presence of base, N-fluoropyridinium triflate, N-fluoroperfluoropiperidine in the presence of base. Chlorinating reagents include, but are not limited to, hexachloroethane in the presence of base, CF₃CF₂CH₂ICl₂, SO₂Cl₂, SOCl₂CF₃SO₂Cl in the presence of base, Cl₂, NaOCl in the presence of acetic acid. Brominating reagents include, but are not limited to, Br₂.pyridine.HBr, Br₂/acetic acid, N-bromosuccinimide in the presence of base, LDA/BrCH₂CH₂Br, or LDA/CBr₄. A suitable iodinating reagent is N-Iodosuccinimide in the presence of base, or I₂, for example. A preferred halogenating reagent is N-fluorobenzenesulfonimide in the presence of sodium hydride.

Suitable bases for the halogenating reactions requiring them are compounds such as alkali metal hydrides, such as NaH and KH, or amine bases, such as LDA or triethylamine, for example. Different reagents may require different type of base, but this is well known within the art.

It shall also be understood that compounds of formula I, wherein X_{H} is fluoro are synthesized from 8-Flurythromycin A in place of erythromycin A according to the synthetic schemes and experimental methods delineated herein.

### EXAMPLES

The compounds and processes of the present invention will be described further in detail with respect to specific preferred embodiments by way of examples, it being understood that these are intended to be illustrative only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1

Compound of formula Ia: A and B taken together with the carbon atom to which they are attached are C═CH₂, L is CH₂CH₃, G is OCH₃

, Q is N(CH₃)₂, and Y taken together with the carbon atom to which they are attached are C═O, R₅ is OBz, R₆ is H, X_{H} is H, and R₂′ is H.

### Step 1a. Compound of formula 1.1 of Scheme 1: G is OCH₃, R₂′ is Bz and R₄″ is Bz

A solution of compound 1.1 of Scheme 1, wherein G is OCH₃, R₂′ and R₄″ are H (prepared according to Elliott et al. J. Med. Chem. 1998, 41, 1651-1659) (95.91 g, 131.51 mmol) in methylene chloride (1 L) containing benzoyl anhydride (90%, 66.26 g, 289.30 mmol), triethylamine (54.81 mL, 433.95 mmol) and DMAP (320 mg, 2.63 mol) was heated to reflux overnight. The resulting mixture was washed with saturated NaHCO₃ solution and brine, concentrated under reduced pressure and recrystalized in acetonitrile to give 77.30 g of the title compound as a white solid.

MS (ESI) m/z=938 (M+H)⁺. ¹³C-NMR (125 MHz, CDCl₃): δ 207.6, 175.2, 166.2, 165.2, 141.2, 138.9, 133.4, 132.5, 130.8, 129.7, 128.4, 128.1, 100.6, 95.9, 80.0, 79.6, 78.9, 78.3, 78.0, 73.2, 72.9, 72.4, 67.7, 63.7, 63.4, 50.6, 49.7, 44.9, 40.9, 39.7, 38.5, 35.4, 31.8, 22.2, 21.7, 21.3, 21.2, 18.7, 18.3, 15.5, 13.7, 10.6, 9.8.

### Step 1b. Compound of formula 1.2 of Scheme 1: G is OCH₃, R₁₁ is H, R₂′ is Bz and R₄″ is Bz

A mixture of the compound from Step 1a (3.40 g, 3.62 mmol), tetrabutylammonium iodide (268 mg, 0.72 mmol), methylene chloride (15.0 mL), propargyl bromide (80% in toluene, 2.42 mL, 21.7 mmol) and sodium hydroxide (50% in water, 15.0 mL) was stirred at room temperature for 3 hours. The mixture was partitioned (ethtyl acetate and water). The organic phases were washed with water and brine, dried (Na₂SO₄) and evaporated. The residue was purified by chromatography (silica, hexanes:acetone/95:5 and 9:1) to give 1.32 g (37%) of the title compound.

MS (ESI) m/z=976 (M+H)⁺. ¹³C-NMR (125 MHz, CDCl₃): δ 205.7, 174.7, 166.1, 165.2, 140.9, 137.2, 133.3, 132.5, 130.8, 129.8, 129.6, 128.4, 128.1, 100.6, 96.1, 80.2, 79.9, 78.9, 78.8, 78.3, 78.1, 76.3, 73.8, 72.8, 72.3, 67.7, 63.7, 63.3, 51.2, 50.7, 49.7, 45.2, 40.8, 39.7, 39.1, 38.4, 35.5, 31.7, 22.2, 21.8, 21.2, 21.1, 19.7, 18.4, 18.3, 16.3, 13.0, 10.2, 9.8.

### Step 1c. Compound 1.4 of Scheme 1: G is OCH₃, R is SnBu₃, R₁₁ is H, R₂′ is Bz and R₄″ is Bz

A solution of the compound from Step 1b (57.6 mg, 0.059 mmol) in anhydrous benzene (5.0 mL) was heated to reflux with tributyltin hydride (82 mg, 0.28 mmol) in the presence of AIBN (2 mg) for 2.5 hours before chromatography (silica, hexanes:acetone/95:5) to give the title compound (46.0 mg, 62%).

MS (ESI) m/z=1266/1268 (M+H)⁺.

### Step 1d. Compound 1.4 of Scheme 1: G is OCH₃, R is H, R₁₁ is H, R₂′ is Bz and R₄″ is Bz

A solution of the compound from Step 1c (46.0 mg, 0.036 mmol) in ethanol (2.0 mL) was treated with hydrochloric acid (2 M, 2.0 mL) at room temperature for 15 minutes. The mixture is partitioned (ethyl acetate and saturated NaHCO₃). The organic phase is washed with water and brine, dried (Na₂SO₄) and evaporated. The residue is purified by chromatography to give the title compound.

MS (ESI) m/z=978 (M+H)⁺.

### Step 1e. Title Compound

A solution of the compound from Step 1d in methanol is refluxed for 24 hours, evaporated and purified by column chromatography to give the title compound.

### Example 2

Compound of formula Ia: A and B taken together with the carbon atom to which they are attached are C═CH₂, L is CH₂CH₃, G is OCH₃, Q is N(CH₃)₂, X and Y taken together with the carbon atom to which they are attached are C═O, R₅ is OH, R₆ is H, X_{H} is H, and R₂′ is H.

The compound of Example 1 is treated with lithium hydroxide in THF at reflux temperature, evaporated and purified by column chromatography to give the title compound.

### Example 3

Compound of formula I: A and B taken together with the carbon atom to which they are attached are C═CH₂, L is CH₂CH₃, G is OCH₃, Q is N(CH₃)₂, X and Y taken together with the carbon atom to which they are attached are C═O. U is OH, V is H, Rₑ is H, R_{f} is CH₃, X_{H} is H, and R₂′ is H.

### Step 3a. Compound 1.6 of Scheme 1: G is OCH₃, R is H, R₁₁ is H, and R₂′ is Bz

A solution of the compound from Step 1c (46.0 mg, 0.036 mmol) in ethanol (2.0 mL) was treated with hydrochloric acid (2 M, 2.0 mL) at 50° C. for 3 hours and 60° C. for 2 hours before partition (ethyl acetate and saturated NaHCO₃). The organic phases were washed with water and brine, dried (Na₂SO₄) and evaporated. The residue was purified by chromatography (silica, hexanes:acetone/95:5˜4:1) to give the title compound (15.1 mg, 58%).

MS (ESI) m/z=716 (M+H)⁺. ¹³C-NMR (125 MHz, CDCl₃): δ 215.7, 174.8, 165.4, 146.2, 132.8, 130.6, 129.9, 129.7, 128.3, 111.3, 99.9, 86.0, 80.7, 78.4, 77.9, 77.0, 72.0, 70.4, 68.9, 63.4, 49.5, 48.6, 45.7, 44.0, 40.8, 37.4, 35.8, 35.6, 32.1, 21.6, 21.1, 19.4, 19.1, 15.13, 15.07, 14.9, 10.3, 7.7.

### Step 3b. Title Compound

A solution of the compound from Step 3a in methanol is refluxed for 24 hours, evaporated and purified by column chromatography to give the title compound.

### Example 4

Compound of formula Ia: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, R₅ is OBz, R₆ is H, X_{H} is H, and R₂′ is H;

### Step 4a. Compound 1.4 of Scheme 1: G is OCH₃, R is —S(CH₂)₂-phenyl, R₁₁ is H, R₂′ is H. and R₄″ is Bz

A solution of the compound from Step 1b (303 mg, 0.31 mmol) in anhydrous benzene (6.2 mL) was heated to reflux with 2-phenylethylthiol (0.10 mL, 0.75 mmol) in the presence of AIBN (8.9 mg) for 21 hours before additional AIBN (3×8.9 mg) was added at every 7˜22 hour intervals during a total of 65 hours reaction time. The solution was evaporated and the residue was chromatographed (silica, hexanes:acetone/98:2˜9:1) to give the title compound (200 mg, 58%).

MS (ESI) m/z=1114 (M+H)⁺.

### Step 4b. Title Compound

A solution of the compound from Step 4a in methanol is refluxed for 24 hours, evaporated and purified by column chromatography to give the title compound.

### Example 5

Compound of formula I: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H.

### Step 5a. Compound 1.6 of Scheme 1: G is OCH₃, R is —S(CH₂)₂-phenyl, R₁₁ is H and R₂′ is Bz

A solution of the compound from Step 4a of Example 4 (200 mg, 0.18 mmol) in ethanol (5.0 mL) was treated with hydrochloric acid (2 M, 5.0 mL) at 60° C. for 2 hours before partition (ethyl acetate and saturated NaHCO₃). The organic phases were washed with water and brine, dried (Na₂SO₄) and evaporated. The residue was chromographed (silica, hexanes:acetone/95:5˜85:15) to give the title compound (81.6 mg, 53%) as a 3:1 isomeric mixture.

MS (ESI) m/z=852 (M+H)⁺.

### Step 5b. Title Compound

A solution of the compound from Step 5a in methanol is refluxed for 24 hours, evaporated and purified by column chromatography to give the title compound.

### Example 6

Compound of formula Ia: A is H, B is —CH₂SC(O)CH₃, L is CH₂CH₃, G is OCH₃, Q is N(CH₃)₂, X and Y taken together with the carbon atom to which they are attached are C═O, R₅ is OBz, R₆ is H, and R₂′ is H.

### Step 6a. Compound of formula 1.3 of Scheme 1: G is OCH₃, R₂′ is Bz, R₄′ is B and R₁₁ is H

Into a mixture of the compound from Step 1a of Example 1 (30.25 g, 32.24 mmol), allyl (tert-butyl)carbonate (6.63 g, 41.92 mmol) and 1,4-bis(diphenylphosphino)butane (931 mg, 2.18 mmol) in freshly distilled THF (200 ml) was added Pd₂(dba)₃ (1.000 g, 1.09 mmol). The reaction mixture was heated to reflux slowly. After refluxing for 16 hours, the mixture was cooled to room temperaure and evapaorated. The residue was purified by silica gel chromatography (hexanes:acetone/98:2˜9:1) and recrystalization (acetonitrile) to give the title compound (28.31 g, 90%).

MS (ESI) m/z: 978 (M+H)⁺¹³C-NMR (125 MHz, CDCl₃): δ 205.1, 174.1, 165.5, 164.6, 139.5, 138.0, 134.2, 132.7, 131.8, 130.2, 129.2, 129.0, 127.7, 127.4, 115.5, 100.0, 95.5, 79.3, 78.3, 77.7, 76.7, 76.4, 76.2, 76.0, 72.2, 71.8, 67.0, 63.2, 63.0, 62.7, 50.1, 49.0, 44.6, 40.2, 39.1, 38.5, 37.6, 34.8, 31.0, 21.6, 21.1, 20.6, 20.5, 19.0, 17.7, 17.6, 15.7, 12.1, 9.7, 9.1.

### Step 6b. Compound of formula Ia: A is H, B is —CH₂SC(O)CH₃, L is CH₂CH₃, G is OCH₃, O is N(CH₃)₂, X and Y taken together with the carbon atom to which they are attached are C═O, R₅ is OBz, R₆ X is H and R₂′ is Bz

A solution of the compound from Step 6a (297 mg, 0.30 mmol) in anhydrous toluene (6.0 mL) was heated to gentle reflux with thiolacetic acid (0.10 mL, 1.40 mmol) in the presence of 2,2′-azobisisobutyronitrile (AIBN, 18.8 mg) for 7 hours before additional AIBN (2×10 mg) was added every 6-14 hours interval during a total of 25 hour reaction. It was evaporated and the residue was chromatographed (silica, hexanes:acetone/97:3˜9:1) to give the title compound (254 mg, 79%) as a 2.5:1 isomeric mixture.

MS (ESI) m/z=1054 (M+H)⁺

### Step 6c. Title Compound

A solution of the compound from Step 6b in methanol is refluxed for 24 hours, evaporated and purified by column and high performance liquid chromatography to give the title compound.

### Example 7

Compound of formula I: A is H, B is —CH₂SC(O)CH₃, L is CH₂CH₃, G is OCH₃, Q is N(CH₃)₂, X and Y taken together with the carbon atom to which they attached are C═O, U is OH, V is H, Rₑ is H, R_{f} is CH₃, X is H, and R₂′ is H.

### Step 7a. Compound of formula I: A is H, B is —CH₂SC(O)CH₃, L is CH₂CH₃, G is OCH₃, Q is N(CH₃)₂, X and Y taken together with the carbon atom to which they attached are C═O, U is OH, V is H, Rₑ is H, R_{f} is CH₃, X_{H} is H, and R₂′ is Bz

A solution of the compound from Step 6b of Example 6 (253 mg, 0.24 mmol) in ethanol (5.0 mL) was treated with hydrochloric acid (2 M, 5.0 mL) at 60° C. for 1.5 hours before partition (ethyl acetate and saturated NaHCO₃). The organic phases were washed with water and brine, dried (Na₂SO₄) and evaporated. The residue was chromographed (silica, hexanes:acetone/95:5˜85:15) to give the title compound (173.5 mg, 91%) as a 2.5:1 isomeric mixture.

MS (ESI) m/z=792 (M+H)⁺.

### Step 7b. Title Compound

A solution of the compound from Step 7a in methanol is refluxed for 24 hours, evaporated and purified by column and high performance liquid chromatography to give the title compound.

### Example 8

Compound of formula I: A is H, B is —CH₂SCH₂-(4-pyridyl), L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H.

### Step 8a. Compound of formula I: A is H, B is —CH₂SH, L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is Bz

A solution of the compound from Step 7a of Example 7 (173.5 mg, 0.22 mmol) in isopropanol (5.0 mL) was treated with aqueous NaOH (10%, 1.0 mL) at room temperature for 6 hours before partition (ethyl acetate and saturated NaHCO₃). The organic phases were washed with water and brine, dried (Na₂SO₄) and evaporated. The residue was chromographed (silica, hexanes:acetone/95:5˜85:15) to give the title compound (67.6 mg, 41%) as a 2.5:1 isomeric mixture.

MS (ESI) m/z=750 (M+H)⁺.

### Step 8b. Compound of formula I: A is H, B is —CH₂SCH₂-(4-pyridyl), L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is Bz

A mixture of the compound from Step 8a, 4-(bromomethyl)pyridine hydrobromide, potassium carbonate in N,N-dimethylformaldehyde is stirred at room temperture for 16 hours before partition (ethyl acetate and saturated NaHCO₃). The organic phases are washed with water and brine, dried (Na₂SO₄), evaporated and chromatographed to give the title compound.

### Step 8c. Title Compound

A solution of the compound from Step 8b in methanol is refluxed for 24 hours, evaporated and chromatographed by column and high performance liquid chromatography to give the title compound.

### Example 9

Compound of formula I: A and B taken together with the carbon atom to which they are attached are C═CHS(CH₂)₂-phenyl, L is CH₂CH₃, G is OCH₃, is N(CH₃)₂, X and Y taken together with the carbon atom to which they are attached are C═O, U is OH, V is H, Rₑ is H, R_{f} is CH₃, X_{H} is H, and R₂′ is H.

### Step 9a. Compound of formula Ib: and L is CH₂CH₃, G is OCH₃, U is OH, V is H, and R₂′ is Bz

A solution of the compound from Step 1b of Example 1 (1.132 g, 1.16 mmol) in ethanol (10 mL) was treated with hydrochloric acid (2 M, 10 mL) at 60° C. for 6 hours before partition (ethyl acetate and saturated NaHCO₃). The organic phases were washed with water and brine, dried (Na₂SO₄) and evaporated. The residue was chromographed (silica, hexanes:acetone/95:5˜7:3) to give the title compound (595 mg, 72%).

MS (ESI) m/z=714 (M+H)⁺. ¹³C-NMR (125 MHz, CDCl₃): δ 206.7, 175.5, 165.5, 141.6, 136.7, 132.6, 130.6, 129.8, 128.1, 102.9, 88.1, 80.0, 79.9, 78.9, 76.6, 76.0, 74.0, 72.2, 69.2, 64.2, 51.5, 48.7, 43.9, 40.7, 37.6, 37.3, 36.9, 30.7, 21.1, 20.2, 19.7, 17.2, 14.5, 13.0, 10.1, 9.9.

### Step 9b. Compound 1.6 of Scheme 1: G is OCH₃, R is —S(CH₂)₂-phenyl, R₁₁ is H and R₂′ is Bz

A solution of the compound from Step 9a in anhydrous toluene is heated to reflux with 2-phenylethylthiol in the presence of AIBN for 3 days according to the procedure described in Step 4a of Example 4. The solution is evaporated and chromatographed to give the title compound.

### Step 9c. Title Compound

A solution of the compound of Step 9b in methanol is refluxed for 24 hours, evaporated and purified by column and high performance liquid chromatography to give the title compound.

### Example 10

Compound of formula I: A and B taken together with the carbon atom to which they are attached are C═CHSC(O)CH₃, L is CH₂CH₃, G is OCH₃, Q is N(CH₃)₂, X and Y taken together with the carbon atom to which they are attached are C═O, U is OH, V is H, Rₑ is H, R_{f} is CH₃, X_{H} is H, and R₂′ is H.

### Step 10a. Compound 1.6 of Scheme 1: G is OCH₃, R is —SC(O)CH₃, R₁₁ is H and R₂′ is Bz

A solution of the compound from Step 9a of Example 9 (505 mg, 0.71 mmol) in anhydrous toluene (14.0 mL) was heated to gentle reflux with thiolacetic acid (0.25 mL, 3.50 mmol) in the presence of 2,2′-azobisisobutyronitrile (AIBN, 22.7 mg) for 8 hours before additional AIBN (2×22 mg) was added every 6˜14 hours interval during a total of 30 hour reaction. It was evaporated and the residue was chromatographed (silica, hexanes:acetone/95:5˜4:1) to give the title compound (307 mg, 55%).

MS (ESI) m/z=790 (M+H)⁺.

### Step 10b. Title Compound

A solution of the compound from Step 10a in methanol is refluxed for 24 hours, evaporated and purified by column and high performance liquid chromatography to give the title compound.

### Example 11

Compound of formula Ia: A and B taken together with the carbon atom to which they are attached are C═CHSCH₂-phenyl, L is CH₂CH₃, G is OCH₃, is N(CH₃)₂, X and Y taken together with the carbon atom to which they are attached are C═O, R₅ is OBz R₆ is H, X_{H} is H, and R₂′ is H.

### Step 11a. Compound 1.1 of Scheme 1: G is OCH₃, R₂ is H, R₂′ is H and R₄″ is Bz

A solution of the compound from Step 1a of Example 1 (5.50 g, 4.80 mmol) in MeOH (200 mL) was refluxed for 16 hours before evaporation. The residue was chromatographed (silica, hexanes:acetone) to give the the title compound (4.85 g, 99%).

MS (ESI) m/z=834 (M+H)⁺. ¹³C-NMR (125 MHz, CDCl₃): δ 205.6, 184.5, 177.7, 171.5, 167.5, 153.4, 139.5, 135.4, 129.6, 127.6, 127.32, 127.0, 102.8, 79.1, 78.9, 76.5, 75.3, 74.4, 70.2, 69.5, 65.8, 62.9, 62.7, 50.5, 46.0, 40.2, 38.5, 28.3, 25.1, 23.6, 21.2, 20.0, 19.2, 17.5, 14.9, 13.8, 13.4, 12.6.

### Step 11b. Compound 1.1 of Scheme 1: G is OCH₃, R₂′ is triethylsilyl and R₄″ is Bz

A solution of the compound from Step 11a (4.87 g, 5.85 mmol), imidazole (2.39 g, 35.14 mmol) and DMAP (150 mg, 1.23 mmol) in DMF (20 mL) was treated with triethylsilyl chloride (1.13 mL, 6.73 mmol) at room temperature for 10 hours. The reaction mixture was diluted with ethyl acetate (200 mL), washed with water and brine, dried and concentrated. The crude residue was purified by chromatography (silica, hexanes:acetone/20:1˜3:1) to give the title compound (5.73 g, 78%).

MS (ESI) m/z=948 (M+H)⁺. ¹³C-NMR (125 MHz, CDCl₃): δ 207.7, 175.3, 166.3, 142.2, 133.2, 129.9, 128.3, 103.1, 96.3, 79.1, 78.4, 73.4, 73.0, 72.7, 67.5, 66.1, 63.4, 50.9, 49.6, 45.2, 41.1, 40.8, 40.5, 35.6, 31.6, 29.3, 22.1, 20.7, 18.4, 14.1, 13.3, 10.6, 7.0, 5.1.

### Step 11c. Compound 1.2 of Scheme 1: G is OCH₃, R₁₁ is H, R₂′ is triethylsilyl and R₄′ is Bz

Into a suspension of the compound from Step 11b (3.25 g, 3.43 mmol), tetrabutylammonium iodide (253 mg, 0.69 mmol) and 50% NaOH aqueous solution (20 mL) in methylene chloride (20 mL) was added propargyl bromide (80% solution in tolune, 1.31 mL, 13.72 mmol) at room temperature. The mixture was stirred vigorously for 18 hours, diluted with ethyl acetate (200 mL),. washed with water and brine, dried (Na₂SO₄) and concentrated. The crude residue was purified by chromatography (silica, hexanes:acetone/20:1) to give the title compound (2.09 g, 62%).

MS (ESI) m/z=986 (M+H)⁺. ¹³C-NMR (125 MHz, CDCl₃): δ 207.5, 174.9, 166.3, 138.3, 133.2, 129.9, 129.8, 128.3, 102.4, 96.7, 80.1, 79.2, 79.0, 78.1, 76.3, 74.0, 73.1, 72.7, 67.5, 66.0, 63.3, 51.5, 51.1, 49.6, 45.6, 39.8, 35.7, 31.6, 29.3, 23.1, 22.6, 21.6, 21.2, 19.8, 18.5, 14.1, 12.7, 10.3, 7.0, 5.1.

### Step 11d. Compound of formula Ia: A and B taken together with the carbon atom to which they are attached are C═CHSCH₂-phenyl, L is CH₂CH₃, G is OCH₃, Q is N(CH₃)₂, X and Y taken together with the carbon atom to which they are attached are C═O, R₅ is OBz, R₆ is H, X_{H} is H, and R₂′ is triethylsilyl

A solution of the compound from Step 11c (200 mg, 0.20 mmol) in toluene (5.0 mL) containing AIBN (3.3 mg, 0.02 mmol) and benzyl mercaptan (0.048 mL, 0.40 mmol) was refluxed for 10 hours. Removal of the solvent by evaopration gave the crude title compound (221 mg).

MS (ESI) m/z=1110 (M+H)⁺.

### Step 11e. Title Compound

A solution of the compound from Step 11d in THF is treated with tetrabutylammonium fluoride at room temperature for 2 hours. Removal of the solvent gives the title compound.

### Example 12

Compound of formula Ia: A and B taken together with the carbon atom to which they are attached are C═O, L is CH₂CH₃, G is OCH₃, Q is N(CH₃)₂, X and Y taken together with the carbon atom to which they are attached are C═O, R is Bz, R₆is H, Rₑ is H, R_{f} is CH₃, X_{H} is H, and R₂′ is H.

### Step 12a. Compound of formula VI:

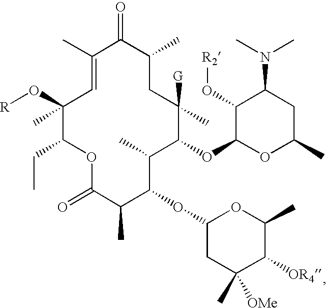

wherein G is OCH₃, R is CH₂CH(OH)CH₂OH, R₂′ is Bz and R₄″ is Bz

A solution of the compound from Step 6a of Example 6 (1.40 g, 1.43 mmol), 4-methylmorpholine N-oxide (503 mg, 4.29 mmol), OsO₄(4 wt % in water, 1.52 mL) in THF (30 mL) and water (7.5 mL) was refluxed overnight, diluted with methylene chloride, washed with saturated NaHCO₃ and brine, dried (Na2SO₄) and concentrated. The residue was purified by chromatography (silica, hexanes:acetone/2:1) to give the title compound (681 mg, 47%).

MS (ESI) m/z=1012 (M+H)⁺.

### Step 12b Compound IV: G is OCH₃, R is CH₂CHO, R₂′ is Bz and R₄′ is Bz

A mixture of the compound from Step 12a (390 mg, 0.39 mmol) and NaIO₄ (82 mg, 0.78 mmol) in THF (6.7 mL) and water (1.6 mL) was stirred at room temperature for 2 hours, diluted with ethyl acetate (50 mL), washed with water (×3) and brine, dried (Na₂SO₄) and concentrated to give the crude title compound (289 mg, 83%).

MS (ESI) m/z=980 (M+H)⁺.

### Step 12c. Compound of formula Ia: A is H, B is OH, L is CH₂CH₃, G is OCH₃, Q is N(CH₃)₂, X and Y taken together with the carbon atom to which they are attached are C═O, R₅ is OBz, R₆ is H, X_{H} is H, and R₂′ is Bz

To a solution of CH₂I₂ (0.048 mL) in THF (1.5 mL) was added Sm (samarium) powder (98 mg) at room temperature. The mixture was stirred at 35° C. until the Sm powder disappeared. Then a solution of the compound from Step 12b (58 mg, 0.059 mmol) in THF (59 mL) was introduced dropwise. After 10 minutes, the reaction was quenched by water (4 mL) and extracted with EtOAc. The extracts were washed with water and brine, dried (Na₂SO₄) and concentrated. The residue was purified by chromatography (silica, hexanes:acetone/2:1) to give the title compound (29 mg, 50%).

MS (ESI) m/z=982 (M+H)⁺.

### Step 12d. Compound of formula Ia: A and B taken together with the carbon atom to which they are attached are C═O, L is CH₂CH₃, G is OCH₃, Q is N(CH₃)₂, X and Y taken together with the carbon atom to which they are attached are C═O, R₅ is OBz, R₆ is H, X_{H} is H, and R₂′ is Bz

A solution of the compound from Step 12c (28 mg, 0.029 mmol) in methylene chloride (0.5 mL) was treated with Dess-Martin periodinane (30 mg, 0.041 mmol) for 40 minutes at room temperature. Evaporation and purification by chromatography (silica, hexanes:acetone/4:1) gave the title compound (19 mg, 68%).

MS (ESI) m/z=980 (M+H)⁺.

### Step 12e. Title Compound

A solution of the compound from Step 12d in methanol is stirred at 55° C. for 24 hours, evaporated and purified by column and high performance liquid chromatography to give the title compound.

### Example 13

Compound of formula Ia: A is H and B is OH, L is CH₂CH₃, G is OCH₃, Q is N(CH₃)₂, X and Y taken together with the carbon atom to which they are attached are C═O, R₅ is OBz, R₆ is H, X_{H} is H, and R₂′ is H.

A solution of the compound from Step 12c in methanol is stirred at 55° C. for 24 hours, evaporated and purified by column and high performance liquid chromatography to give the title compound.

### Example 14

Compound of formula I: A and B taken together with the carbon atom to which they are attached are C═CH₂, L is CH₂CH₃, G is OCH₃, Q is N(CH₃ )₂, X and Y taken together with the carbon atom to which they are attached are C═O, U and V taken together with the carbon atom to which they are attached are C═O, Rₑ is H, R_{f} is CH₃, is X_{H} and R₂′ is H.

### Step 14a. Compound of formula 1.1 of Scheme 1: G is OCH₃, R₂′ is Bz and R₄″ is Bz

A solution of compound 1.1 of Scheme 1, wherein G is OCH₃, and R₂′ and R₄″ are H (prepared according to Elliott et al. J. Med. Chem. 1998, 41, 1651-1659) (95.91 g, 131.51 mmol) in methylene chloride (1 L) containing benzoyl anhydride (90%, 66.26 g, 289.30 mmol), triethylamine (54.81 mL, 433.95 mmol) and DMAP (320 mg, 2.63 mol) was heated to reflux overnight. The resulting mixture was washed with saturated NaHCO₃ solution and brine, concentrated under reduced pressure and recrystalized in acetonitrile to give 77.30 g of the title compound as a white solid.

MS (ESI) m/z=938 (M+H)⁺. ¹³C-NMR (125 MHz, CDCl₃): δ 207.6, 175.2, 166.2, 165.2, 141.2, 138.9, 133.4, 132.5, 130.8, 129.7, 128.4, 128.1, 100.6, 95.9, 80.0, 79.6, 78.9, 78.3, 78.0, 73.2, 72.9, 72.4, 67.7, 63.7, 63.4, 50.6, 49.7, 44.9, 40.9, 39.7, 38.5, 35.4, 31.8, 22.2, 21.7, 21.3, 21.2, 18.7, 18.3, 15.5, 13.7, 10.6, 9.8.

### Step 14b. Compound of formula 1.2 of Scheme 1: G is OCH₃, R₁₁ is H, R₂′ is Bz and R₄″ is Bz

A mixture of the compound from Step 14a (3.40 g, 3.62 mmol), tetrabutylammonium iodide (268 mg, 0.72 mmol), methylene chloride (15.0 mL), propargyl bromide (80% in toluene, 2.42 mL, 21.7 mmol) and sodium hydroxide (50% in water, 15.0 mL) was stirred at room temperature for 3 hours. The mixture was partitioned (ethyl acetate and water). The organic phases were washed with water and brine, dried (Na₂SO₄) and evaporated. The residue was purified by chromatography (silica, hexanes:acetone/95:5 and 9:1) to give 1.32 g (37%) of the title compound.

MS (ESI) m/z=976 (M+H)⁺. ¹³C-NMR (125 MHz, CDCl₃): δ 205.7, 174.7, 166.1, 165.2, 140.9, 137.2, 133.3, 132.5, 130.8, 129.8, 129.6, 128.4, 128.1, 100.6, 96.1, 80.2, 79.9, 78.9, 78.8, 78.3, 78.1, 76.3, 73.8, 72.8, 72.3, 67.7, 63.7, 63.3, 51.2, 50.7, 49.7, 45.2, 40.8, 39.7, 39.1, 38.4, 35.5, 31.7, 22.2, 21.8, 21.2, 21.1, 19.7, 18.4, 18.3, 16.3, 13.0, 10.2, 9.8.

### Step 14c. Compound 1.4 of Scheme 1: G is OCH₃, R is SnBu₃, R₁₁ is H, R₂′ is Bz and R₄″ is Bz

A solution of the compound from Step 14b (57.6 mg, 0.059 mmol) in anhydrous benzene (5.0 mL) was heated to reflux with tributyltin hydride (82 mg, 0.28 mmol) in the presence of AIBN (2 mg) for 2.5 hours before chromatography (silica, hexanes:acetone/95:5) to give the title compound (46.0 mg, 62%).

MS (ESI) m/z ═1266/1268 (M+H)⁺.

### Step 14d. Compound 1.6 of Scheme 1: G is OCH₃, R is H, R₁₁ is H and R₂′ is Bz

A solution of the compound from Step 14c (46.0 mg, 0.036 mmol) in ethanol (2.0 mL) was treated with hydrochloric acid (2 M, 2.0 mL) at 50° C. for 3 hours and 60° C. for 2 hours. The mixture was partitioned (ethyl acetate and saturated NaHCO₃). The organic phases were washed with water and brine, dried (Na₂SO₄) and evaporated. The residue was purified by chromatography to give the title compound (15.1 mg, 58%).

MS (ESI) m/z=716 (M+H)⁺. ¹³C-NMR (125 MHz, CDCl₃): δ 215.7, 174.8, 165.4, 146.2, 132.8, 130.6, 129.9, 129.7, 128.3, 111.3, 99.9, 86.0, 80.7, 78.4, 77.9, 77.0, 72.0, 70.4, 68.9, 63.4, 49.5, 48.6, 45.7, 44.0, 40.8, 37.4, 35.8, 35.6, 32.1, 21.6, 21.1, 19.4, 19.1, 15.13, 15.07, 14.9, 10.3, 7.7.

### Step 14e. Compound 2.1 of Scheme 2: G is OCH₃, R is H, R₁₁ is H and R₂′ is Bz

To a solution of the compound from Step 14d (15.0 mg, 0.021 mmol) in dichloromethane (1.0 mL) was added Dess-Martin periodinane (17.8 mg, 0.042 mmol) at room temperature. The mixture was stirred at room temperature for 3 hours before partition with ethyl acetate and saturated sodium bicarbonate-saturated sodium thiosulfate (3:1). The organic phases were washed with water and brine, dried (Na₂SO₄) and evaporated to give the crude title compound (15 mg).

MS (ESI) m/z 714 (M+H)⁺.

### Step 14f. Title Compound

A solution of the compound from Step 14e (15 mg, 0.02 mmol) in methanol (3 mL) was refluxed for 20 hours and then evaporated. The residue was purified by column chromatography (silica, CH₂Cl₂:2M ammonia in methanol/99:1˜97:3) to give the title compound (12.8 mg, 100% for two steps).

MS (ESI) m/z 610 (M+H)⁺. ¹³C NMR(CDCl₃): δ 216.1, 205.3, 169.6,146.2, 111.4, 103.3, 86.1, 78.11, 78.08, 77.8, 70.6, 70.3, 69.3, 66.0, 51.1, 49.5, 48.3, 46.5, 44.9, 40.3, 38.4, 36.1, 28.6, 21.9, 21.2, 19.7, 18.8, 15.5, 14.64, 14.57, 14.5, 10.5.

### Example 15

Compound of formula I: A and B taken together with the carbon atom to which they are attached are C═CHS(CH₂)₂-phenyl, L is CH₂CH₃, G is OCH₃, Q is N(CH)₂, X and Y taken together with the carbon atom to which they are attached are C═O, U and V taken together with the carbon atom to which they are attached are C═O, Rₑ is H, R_{f} is CH₃, X is H, and R₂′ is H.

### Step 15a. Compound 1.4 of Scheme 1: G is OCH₃, H, R₁₁ is —S(CH₂)₂-phenyl, R₂′ is Bz and R₄″ is Bz

A solution of the compound from Step 14b of Example 14 (303 mg, 0.31 mmol) in anhydrous benzene (6.2 mL) was heated to reflux with 2-phenylethylthiol (0.10 mL, 0.75 mmol) in the presence of AIBN (8.9 mg) for 21 hours before additional AIBN (3×8.9 mg) was added at every 7˜22 hour intervals during a total of 65 hours reaction time. The solution was evaporated and the residue was chromatographed (silica, hexanes:acetone/98:2˜9:1) to give the title compound (200 mg, 58%).

MS (ESI) m/z=1114 (M+H)⁺.

### Step 15b. Compound 1.6 of Scheme 1: G is OCH₃, R is H, R₁₁ is —S(CH₂)₂-phenyl and R₂′ is Bz

A solution of the compound from Step 15a (200 mg, 0.18 mmol) in ethanol (5.0 mL) was treated with hydrochloric acid (2 M, 5.0 mL) at 60° C. for 2 hours before partition (ethyl acetate and saturated NaHCO₃). The organic phases were washed with water and brine, dried (Na₂SO₄) and evaporated. The residue was chromographed (silica, hexanes:acetone/95:5˜85:15) to give the title compound (81.6 mg, 53%) as a 3:1 isomeric mixture.

MS (ESI) m/z=852 (M+H)⁺.

### Step 15c. Compound 2.1 of Scheme 2: G is OCH₃, R is H, R₁₁ is —S(CH₂)₂-phenyl and R₂′ is Bz

Dimethyl sulfide (17.2 μL, 0.23 mmol) was added into a solution of N-chlorosuccinimide (NCS) (25.1 mg, 0.19 mmol) in CH₂Cl₂ (3.0 mL) at −10 ° C. Stirring was continued for 10 minutes before a solution of the compound from Step 15b (80 mg, 0.094 mmol) in CH₂Cl₂ (2.0 mL) was introduced over 5 minutes. After the mixture was stirred at −10 to −5° C. for 1 hour, triethylamine (13.1 μL, 0.094 mmol) was charged and the mixture was stirred for another 45 minutes before warming to room temperature and being partitioned (ethyl acetate and saturated NaHCO₃). The organic phases were washed with water and brine, dried (Na₂SO₄) and evaporated. The residue was chromographed (silica, hexanes:acetone/95:5˜85:15) to give the title compound (36.2 mg, 45%).

MS (ESI) m/z=850 (M+H)⁺. ¹³C-NMR (125 MHz, CDCl₃): δ 216.3, 205.5, 169.4, 165.1, 140.2, 138.0, 132.9, 130.3, 129.7, 128.6, 128.33, 128.31, 126.2, 120.3, 101.1, 86.1, 78.1, 77.8, 76.8, 71.7, 69.4, 69.0, 63.6, 53.7, 51.0, 49.3, 48.9, 45.9, 45.3, 40.7, 38.1, 36.6, 36.0, 35.7, 31.7, 21.8, 21.0, 19.6, 18.6, 15.4, 14.8, 14.4, 14.3, 10.5.

### Step 15d. Title Compound

A solution of the compound from Step 15c (36 mg, 0.04 mmol) in methanol (2 mL) was refluxed for 7 hours and then evaporated. The residue was purified by column chromatography (silica, CH₂Cl₂:2M ammonia in methanol/99:1˜97:3) to give the title compound (30.0 mg, 95%).

MS (ESI) m/z 746 (M+H)⁺. ¹³C NMR (CDCl₃): δ 216.4, 205.3, 169.5, 140.2, 138.1, 128.6, 128.3, 126.2, 120.3, 103.3, 86.2, 78.02, 77.95, 77.91, 70.2, 69.4, 69.3, 66.0, 51.1,49.4, 48.9, 46.6, 45.2, 40.2, 38.5, 36.6, 36.2, 35.7, 29.2, 28.7, 21.8, 21.1, 19.6, 18.6, 15.5, 14.8, 14.7, 14.5, 10.5.

### Example 16

Compound of formula I: A and B taken together with the carbon atom to which they are attached are C═CHSO(CH₂)₂-phenyl, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 16a. Compound 2.1 of Scheme 2: G is OCH₃, R is H, R₁₁ is —SO(CH₂)₂-phenyl and R₂′ is Bz

To a solution of the compound from Step 15b (81.6 mg, 0.096 mmol) in dichloromethane (3.0 mL) was added Dess-Martin periodinane (61.1 mg, 0.14 mmol) at room temperature. The mixture was stirred at room temperature for 2.5 hours before partition with ethyl acetate and saturated sodium bicarbonate—saturated sodium thiosulfate (3:1). The organic phases were washed with water and brine, dried (Na₂SO₄) and chromographed (silica, hexanes:acetone/95:5˜4: 1) to give the title compound (30.0 mg, 36%).

MS (ESI) m/z 866 (M+H)⁺.

### Step 16b. Title Compound

A solution of the compound from Step 16a (30 mg, 0.035 mmol) in methanol (2 mL) was refluxed for 14 hours and then evaporated. The residue was purified by column chromatography (silica, CH₂Cl₂:2M ammonia in methanol/99:1˜97:3) to give the title compound as 1.5:1 diastereomeric mixture (17.5 mg, 66%).

MS (ESI) m/z 762 (M+H)⁺. ¹³C NMR (CDCl₃) for major isomer (selected data): δ 129.2, 128.8, 126.8, 120.3, 103.3, 77.85, 77.79, 70.3, 69.4, 67.9, 66.3, 55.1, 51.2, 49.7, 46.6, 45.5, 40.5, 38.7, 35.6, 28.9, 28.1, 21.8, 21.3, 19.9, 18.7, 15.5, 15.0, 14.7, 10.6; for minor isomer (selected data): 129.8, 128.9, 126.3, 120.3, 103.6, 77.85, 77.79, 70.3, 69.4, 68.3, 66.3, 54.8, 50.1,49.4, 46.6, 45.5, 40.5, 38.7, 35.6, 28.9, 28.1, 21.8, 21.3, 19.9, 18.7, 15.5, 15.0, 14.7, 10.6.

### Example 17

Compound of formula I: A is H, B is —CH₂SC(O)CH₃, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 17a. Compound of formula 1.3 of Scheme 1: G is OCH₃, R₂′ is Bz, R₄″ is Bz and R₁₁ is H

Into a mixture of the compound from Step 14a of Example 1 (30.25 g, 32.24 mmol), allyl (tert-butyl)carbonate (6.63 g, 41.92 mmol) and 1,4-bis(diphenylphosphino)butane (931 mg, 2.18 mmol) in freshly distilled THF (200 ml) was added Pd₂(dba)₃ (1.000 g, 1.09 mmol). The reaction mixture was heated to reflux slowly. After refluxing for 16 hours, the mixture was cooled to room temperaure and evapaorated. The residue was purified by silica gel chromatography (hexanes:acetone/98:2˜9:1) and recrystalization (acetonitrile) to give the title compound (28.31 g, 90%).

MS (ESI) m/z: 978 (M+H)⁺. ¹³C-NMR (125 MHz, CDCl₃): δ 205.1, 174.1, 165.5, 164.6, 139.5, 138.0, 134.2, 132.7, 131.8, 130.2, 129.2, 129.0, 127.7, 127.4, 115.5, 100.0, 95.5, 79.3, 78.3, 77.7, 76.7, 76.4, 76.2, 76.0, 72.2, 71.8, 67.0, 63.2, 63.0, 62.7, 50.1,49.0, 44.6, 40.2, 39.1, 38.5, 37.6, 34.8, 31.0, 21.6, 21.1, 20.6, 20.5, 19.0, 17.7, 17.6, 15.7, 12.1, 9.7, 9.1.

### Step 17b. Compound 1.5 of Scheme 1: G is OCH₃, R is H, R₁₁ is —SC(O)CH₃, R₂′ is Bz and R₄″ is Bz

A solution of the compound from Step 17a (297 mg, 0.30 mmol) in anhydrous toluene (6.0 mL) was heated to gentle reflux with thiolacetic acid (0.10 mL, 1.40 mmol) in the presence of 2,2′-azobisisobutyronitrile (AIBN, 18.8 mg) for 7 hours before additional AIBN (2×10 mg) was added every 6˜14 hours interval during a total of 25 hour reaction. It was evaporated and the residue was chromatographed (silica, hexanes:acetone/97:3˜9:1) to give the title compound (254 mg, 79%) as a 2.5:1 isomeric mixture.

MS (ESI) m/z=1054 (M+H)⁺.

### Step 17c. Compound 1.7 of Scheme 1: G is OCH₃, R is H, R₁₁ is —SC(O)CH₃ and R₂′ is Bz

A solution of the compound from Step 17b (253 mg, 0.24 mmol) in ethanol (5.0 mL) was treated with hydrochloric acid (2 M, 5.0 mL) at 60° C. for 1.5 hours before partition (ethyl acetate and saturated NaHCO₃). The organic phases were washed with water and brine, dried (Na₂SO₄) and evaporated. The residue was chromographed (silica, hexanes:acetone/95:5˜85:15) to give the title compound (173.5 mg, 91%) as a 2.5:1 isomeric mixture.

MS (ESI) m/z=792 (M+H)⁺.

### Step 17d. Compound 2.2 of Scheme 2: G is OCH₃, R is H, R₁₁ is —SC(O)CH₃ and R₂′ is Bz

The title compound is prepared from the compound of Step 4c using Dess-Martin Periodinane according to the procedure described in Example 1 (Step 1e) or NCS and dimethyl sulfide according to Example 2 (Step 2c).

### Step 17e. Title Compound

A solution of the compound from Step 17d in methanol is refluxed for 24 hours, evaporated and the residue is purified by chromatography to give the title compound.

### Example 18

Compound of formula I: A and B taken together with the carbon atom to which they are attached are C═CHS(CH₂)₂-phenyl, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 18a. Compound of formula Ib: G is OCH₃, U is OH, V is H, R₂′ is Bz

A solution of the compound from Step 14b (1.132 g, 1.16 mmol) in ethanol (10 mL) was treated with hydrochloric acid (2 M, 10 mL) at 60° C. for 6 hours before partition (ethyl acetate and saturated NaHCO₃). The organic phases were washed with water and brine, dried (Na₂SO₄) and evaporated. The residue was chromographed (silica, hexanes:acetone/95:5˜7:3) to give the title compound (595 mg, 72%).

MS (ESI) m/z=714 (M+H)⁺. ¹³C-NMR (125 MHz, CDCl₃): δ 206.7, 175.5, 165.5, 141.6, 136.7, 132.6, 130.6, 129.8, 128.1, 102.9, 88.1, 80.0, 79.9, 78.9, 76.6, 76.0, 74.0, 72.2, 69.2, 64.2, 51.5, 48.7, 43.9, 40.7, 37.6, 37.3, 36.9, 30.7, 21.1, 20.2, 19.7, 17.2, 14.5, 13.0, 10.1, 9.9.

### Step 18b. Compound 1.6 of Scheme 1: G is OCH₃, R is —S(CH₂)₂-phenyl, R₁₁ is H and R₂′ is Bz

A solution of the compound from Step 18a in anhydrous toluene is heated to reflux with 2-phenylethylthiol in the presence of AIBN for 3 days according to the procedure described in Example 15 (Step 15a). The solution is evaporated and the residue is chromatographed to give the title compound.

### Step 18c. Compound 2.1 of Scheme 2: G is OCH₃, R is H, R₁₁ is —S(CH₂)₂-phenyl and R₂′ is Bz

The title compound is prepared from the compound of step 18b using NCS and dimethyl sulfide according to the procedure described in Example 15 (Step 15c).

### Step 18d. Title Compound

A solution of the compound from Step 18c in methanol is refluxed for 24 hours, evaporated and the residue purified by column and high performance liquid chromatography to give the title compound.

### Example 19

Compound of formula I: A and B taken together with the carbon atom to which they are attached are C═CHSC(O)CH₃, L is CH₂CH₃, G is OCH₃, is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 19a. Compound 1.6 of Scheme 1: G is OCH₃, R is —SC(O)CH₃, R₁₁ is H and R₂′ is Bz

A solution of the compound from step 18a (505 mg, 0.71 mmol) in anhydrous toluene (14.0 mL) was heated to gentle reflux with thiolacetic acid (0.25 mL, 3.50 mmol) in the presence of 2,2′-azobisisobutyronitrile (AIBN, 22.7 mg) for 8 hours before additional AIBN (2×22 mg) was added every 6˜14 hours interval during a total of 30 hour reaction. It was evaporated and the residue was chromatographed (silica, hexanes:acetone/95:5˜4:1) to give the title compound (307 mg, 55%).

MS (ESI) m/z=790 (M+H)⁺.

### Step 19b. Compound 2.1 of Scheme 2: G is OCH₃, R is H, R₁₁ is —SC(O)CH₃ and R₂′ is Bz

The title compound is prepared from the compound of Step 19a using Dess-Martin Periodinane according to the procedure described in Example 14 (Step 14e) or NCS and dimethyl sulfide as described in Example 15 (Step 15c).

### Step 19c. Title Compound

A solution of the compound from Step 19b in methanol is refluxed for 24 hours, evaporated and purified by column and high performance liquid chromatography to give the title compound as one of the C10 stereoisomers.

### Example 20

Compound of formula I: A and B taken together with the carbon atom to which they are attached are C═CHSC(O)CH₃, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃X_{H} is H, and R₂′ is H.

### Step 20a. Compound of formula Ib: G is OCH₃, R₂′ is Bz, U and V taken together with the carbon atom to which they are attached are C═O

Into a solution of the compound from Step 18a (595 mg, 0.83 mmol) in dichloromethane (5.0 mL) was added Dess-Martin periodinane (424 mg, 1.00 mmol) at room temperature. The mixture was stirred at room temperature for 3.5 hours before partition with ethyl acetate and saturated sodium bicarbonate-saturated sodium thiosulfate (3: 1). The organic phases were washed with water and brine, dried (Na₂SO₄) and evaporated to give the crude title compound (472 mg, 80%).

MS (ESI) m/z 712 (M+H)⁺. ¹³C-NMR (125 MHz, CDCl₃): δ 205.6, 203.6, 169.4, 165.1, 140.8, 137.9, 132.8, 130.3, 129.7, 128.2, 101.9, 81.3, 80.2, 78.7, 78.0, 77.7, 77.1, 73.9, 71.8, 69.1, 63.6, 51.3, 51.0, 50.3, 46.8, 40.7, 40.3, 39.2, 31.2, 22.1, 20.9, 19.9, 18.9, 14.7, 14.1, 12.9, 10.4.

### Step 20b. Compound 2.1 of Scheme 2: G is OCH₃, R is H, R₁₁ is —SC(O)CH₃ and R₂′ is Bz

A solution of the compound from Step 20a (210 mg, 0.29 mmol) in anhydrous benzene (6.0 mL) was heated to gentle reflux with thiolacetic acid (0.042 mL, 0.59 mmol) in the presence of 2,2′-azobisisobutyronitrile (AIBN, 15.0 mg) for 8 hours before additional AIBN (8×6 mg) was added every 6˜14 hours interval during a total of 10 days reaction while additional thiolacetic acid (0.20 mL) was added in day 8. It was evaporated and the residue was chromatographed (silica, hexanes:acetone/95:5˜85:15) to give the title compound (184 mg, 79%).

MS (ESI) m/z=788 (M+H)⁺.

### Step 20c. Title Compound

A solution of the compound from Step 20b in methanol is refluxed for 24 hours, evaporated and purified by column and high performance liquid chromatography to give the title compound as one of the CIO stereoisomers.

### Example 21

Compound of formula I: A and B taken together with the carbon atom to which they are attached are C═CHSCH₂-phenyl, L is CH₂CH₃, G is OCH₃, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 21 a. Compound of formula 1.1 of Scheme 1: G is OCH₃, R₂′ is H and R₄″ is Bz

A solution of the compound from Step 14a of Example 14 (5.50 g, 4.80 mmol) in MeOH (200 mL) was refluxed for 16 hours before evaporation. The residue was chromatographed (silica, hexanes:acetone) to give the the title compound (4.85 g, 99%).

MS (ESI) m/z=834 (M+H)⁺. ¹³C-NMR (125 MHz, CDCl₃): δ 205.6, 184.5, 177.7, 171.5, 167.5, 153.4, 139.5, 135.4, 129.6, 127.6, 127.32, 127.0, 102.8, 79.1, 78.9, 76.5, 75.3, 74.4, 70.2, 69.5, 65.8, 62.9, 62.7, 50.5, 46.0, 40.2, 38.5, 28.3, 25.1, 23.6, 21.2, 20.0, 19.2, 17.5, 14.9, 13.8, 13.4, 12.6.

### Step 21b. Compound of formula 1.1 of Scheme 1: G is OCH₃, R₂′ is triethylsilyl and R₄″ is Bz

A solution of the compound from Step 21a (4.87 g, 5.85 mmol), imidazole (2.39 g, 35.14 mmol) and DMAP (150 mg, 1.23 mmol) in DMF (20 mL) was treated with triethylsilyl chloride (1.13 mL, 6.73 mmol) at room temperature for 10 hours, diluted with ethyl acetate (200 mL), washed with water and brine, dried and concentrated. The crude residue was purified by chromatography (silica, hexanes:acetone/20:1˜3:1) to give the title compound (5.73 g, 78%).

MS (ESI) m/z=948 (M+H)⁺. ¹³C-NMR (125 MHz, CDCl₃): δ 207.7, 175.3, 166.3, 142.2, 133.2, 129.9, 128.3, 103.1, 96.3, 79.1, 78.4, 73.4, 73.0, 72.7, 67.5, 66.1, 63.4, 50.9, 49.6, 45.2, 41.1,40.8, 40.5, 35.6, 31.6, 29.3, 22.1, 20.7, 18.4, 14.1, 13.3, 10.6, 7.0, 5.1.

### Step 21c. Compound of formula 1.2 of Scheme 1: G is OCH₃, R ₁₁ is H, R₂′ is triethylsilyl and R₄″ is Bz

To a suspension of the compound from Step 21b (3.25 g, 3.43 mmol), tetrabutylammonium iodide (253 mg, 0.69 mmol) and 50% NaOH aqueous solution (20 mL) in methylene chloride (20 mL) was added propargyl bromide (80% solution in tolune, 1.31 mL, 13.72 mmol) at room temperature. The mixture was stirred vigorously for 18 hours, diluted with ethyl acetate (200 mL),. washed with water and brine, dried (Na₂SO₄) and concentrated. The crude residue was purified by chromatography (silica, hexanes:acetone/20:1) to give the title compound (2.09 g, 62%).

MS (ESI) m/z=986 (M+H)⁺. ¹³C-NMR (125 MHz, CDCl₃): δ 207.5, 174.9, 166.3, 138.3, 133.2, 129.9, 129.8, 128.3, 102.4, 96.7, 80.1, 79.2, 79.0, 78.1, 76.3, 74.0, 73.1, 72.7, 67.5, 66.0, 63.3, 51.5, 51.1, 49.6, 45.6, 39.8, 35.7, 31.6, 29.3, 23.1, 22.6, 21.6, 21.2, 19.8, 18.5, 14.1, 12.7, 10.3, 7.0, 5.1.

### Step 21d. Compound of formula 1.2 of Scheme 1: G is OCH₃, R₁₁ is H, R₂′ is H and R₄′ is Bz

A solution of the compound from Step 21c (2.02 g, 2.05 mmol) in EtOH (20 mL) and aqueous HCl (2 M, 20 mL) was heated to 50° C. for 4 hours. After removal of EtOH by evaporation, the residue was basified by NaOH (2 M) at 0° C. to pH˜13 and extracted with methylene chloride. The extracts were dried and concentrated. The crude was purified by chromatography (silica, hexanes:acetone/1:2) to give the title compound (957 mg, 77%).

MS (ESI) m/z=610 (M+H)⁺. ¹³C-NMR (125 MHz, CDCl₃): δ 207.1, 176.6, 141.5, 136.8, 106.8, 91.9, 80.8, 80.0, 79. 1, 77.4, 76.1, 74.1, 70.5, 69.7, 65.5, 51.8, 48.3, 44.2, 40.2, 38.3, 36.9, 36.5, 28.2, 21.3, 20.9, 20.4, 19.8, 16.1, 16.0, 12.9, 10.2, 7.6.

### Step 21e. Compound of formula Ib: G is OCH₃, U and V taken together with the carbon atom to which they are attached are C═O and R₂′ is H

A solution of the compound from Step 21d (900 mg, 1.48 mmol) in methylene chloride (15 mL) was treated with Dess-Martin periodinane (900 mg, 2.07 mmol) at room temperature for 3 hours. The solution was diluted with methylene chloride (100 mL), washed with saturated Na₂SO₃, saturated NaHCO₃ and brine, dried (Na₂SO₄) and concentrated. The crude residue was purified by chromatography (silica, 2M NH₃ in MeOH:CH₂Cl₂/1:39) to give the title compound (533 mg, 59%).

MS (ESI) m/z=608 (M+H)⁺. ¹³C-NMR (125 MHz, CDCl₃): δ 205.4, 203.5, 169.6, 140.3, 138.6, 104.2, 138.6, 104.2, 80.2, 78.7, 78.0, 77.6, 74.0, 70.3, 69.5, 65.7, 51.4, 51.1, 50.4, 47.1,40.2, 38.5, 31.5, 28.2, 21.1, 20.9, 18.6, 14.7, 12.8, 10.5.

### Step 21f. Title Compound

A solution of the compound from Step 21e (50.0 mg, 0.082 mmol) in benzene (1.0 mL) containing AIBN (4 mg) and benzyl mercaptan (0.019 mL, 0.16 mmol) was refluxed for 20 hours. Removal of the solvent by evaporation gave the crude title compound (68 mg).

MS (ESI) m/z=732 (M+H)⁺

### Example 22

Compound of formula Ia: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, R₅ is OAc, R₆ is H X_{H} is H, and R₂′ is H.

### Step 22a. Compound 1.2 of Scheme 1: G is OCH₂CH═CH₂, R₁₁ is H, R₄″ is C(O)CH₃ and R₂′ is C(O)CH₃

A mixture of 2′,4″-bis-O-acetyl-6-O-allyl-11-deoxy-10,11-didehydroerythromycin (640 mg, 0.76 mmol), tetrabutylammonium iodide (56 mg, 0.15 mmol), methylene chloride (4.0 mL), propargyl bromide (80% in toluene, 0.68 mL, 6.09 mmol) and sodium hydroxide (50% in water, 6.0 mL) was stirred at room temperature for 2 hours. The mixture was partitioned (ethtyl acetate and water). The organic phases were washed with water and brine, dried (Na₂SO₄) and evaporated. The residue was purified by chromatography (silica, hexanes:acetone/95:5 and 3:1) to give 258 mg (39%) of the title compound.

MS (ESI) m/z=878 (M+H)⁺.

### Step 22b. Compound 1.4 of Scheme 1: R and R₁₁ taken together with the carbon atom to which they are attached are CHSnBu₃, G is OCH₂CH═CH₂, R₂′ is C(O)CH₃ and R₄″ is C(O)CH₃

A solution of the compound from Step 22a (250 mg, 0.28 mmol) in anhydrous benzene (5.7 mL) was heated to reflux with tributyltin hydride (249 mg, 0.85 mmol) in the presence of AIBN (11.5 mg) for 2 hours before evaporation. The residue was chromatographed (silica, hexanes:acetone/95:5˜9:1) to give the title compound (163.5 mg, 49%).

MS (ESI) m/z=1168/1170 (M+H)⁺.

### Step 22c. Compound of formula Ia: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, R₅ is OC(O)CH₃, R₆ is H, X_{H} is H, and R₂′ is C(O)CH₃

A solution of the compound from Step 22b in ethanol is treated with hydrochloric acid at room temperature for 15 minutes. The mixture is partitioned (ethyl acetate and saturated NaHCO₃). The organic phase is washed with water and brine, dried (Na₂SO₄), evaporated and purified by column chromatography to give the title compound

### Step 22d. Title Compound

A solution of the compound from Step 22c in methanol is refluxed for 24 hours. Evaporated and purified by column chromatography to give the title compound.

### Example 23

Compound of formula Ia: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, R₅ is OH, R₆ is H, X_{H} is H, and R₂′ is H.

The compound of Example 22 is treated with lithium hydroxide in THF at reflux temperature to provide the title compound.

### Example 24

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U is OH, V is H, Rₑ is H, R_{f} is CH₃, X_{H} is H, and R₂′ is H.

### Step 24a. Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U is OH, V is H, Rₑ is H, R_{f} is CH₃, X_{H} is H, and R₂′ is C(O)CH₃

A solution of the compound from Step 22b (163.5 mg, 0.14 mmol) in ethanol (4.0 mL) was treated with hydrochloric acid (2 M, 4.0 mL) at 60° C. for 2 hours before partition (ethyl acetate and saturated NaHCO₃). The organic phases were washed with water and brine, dried (Na₂SO₄) and evaporated. The residue was purified by chromatography (silica, hexanes:acetone/95:5˜4:1) to give the title compound (60.3 mg, 63%) as one of the C10 stereoisomers.

MS (ESI) m/z=680 (M+H)⁺. ¹³C NMR (125 MHz, CDCl₃): δ 215.7, 174.7, 145.8, 136.4, 116.3, 112.1, 100.1, 86.2, 81.1, 79.7, 77.6, 77.4, 71.6, 70.7, 68.8, 64.6, 63.5, 48.7, 45.4, 44.3, 40.6, 37.3, 36.5, 35.9, 31.3, 22.0, 21.1, 20.4, 19.5, 15.5, 15.3, 14.8, 10.5, 8.1.

### Step 24b. Title Compound

A solution of the compound of Step 24a in methanol is refluxed for 24 hours, evaporated and purified by column chromatography to give the title compound.

### Example 25

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U is OH, V is H, Rₑ is H, R_{f} is CH₃, X_{H} is H, and R₂′ is H.

### Step 25a. Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U is OH, V is H, Rₑ is H, R_{f} is CH₃, X_{H} is H, and R₂′ is C(O)CH₃

A solution of the compound from Step 22b (163.5 mg, 0.14 mmol) in ethanol (4.0 mL) was treated with hydrochloric acid (2 M, 4.0 mL) at 60° C. for 2 hours before partition (ethyl acetate and saturated NaHCO₃). The organic phases were washed with water and brine, dried (Na₂SO₄) and evaporated. The residue was purified by chromatography (silica, hexanes:acetone/95:5˜4:1) to give the title compound (24.9 mg, 26%) as one of the C10 stereoisomers.

MS (ESI) m/z=680 (M+H)⁺. ¹³C NMR (125 MHz, CDCl₃): δ 215.8, 176.7, 152.3, 136.3, 115.2, 107.6, 104.0, 92.2, 86.1, 80.8, 77.2, 72.1, 69.2, 64.7, 64.6, 61.8, 51.1,48.4, 44.6, 40.8, 39.3, 38.1, 34.8, 29.7, 21.4, 21.2, 20.9, 19.8, 19.6, 15.4, 10.9, 10.7, 8.1.

### Step 25b. Title Compound

A solution of the compound of Step 25a in methanol is refluxed for 24 hours, evaporated and purified by column chromatography to give the title compound.

### Example 26

Compound of formula Ia: A and B taken together with the carbon atom to which they are attached are C═CH₂, L is CH₂CH₃, G is OCH₂CH(O), Q is N(CH₃)₂, X and Y taken together with the carbon atom to which they are attached are C═O, R₂′ is H, X_{H} is H, R₅ is OH, and R₆ is H.

### Step 26a. Compound of formula Ia: A and B taken together with the carbon atom to which they are attached are C═CH₂, L is CH₂CH₃, G is OCH₂CH(O), Q is N(CH₃)₂, X and Y taken together with the carbon atom to which they are attached are C═O, R₂′ is Ac, X_{H} is H, R₅ is OAc, and R₆ is H

To a solution of the compound of Example 23 in aqueous acetone at 25° C. is added OsO₄ (5 mol %) followed by NaIO₄ (4 equivalents) and the mixture is stirred for 4-6 hours. The reaction mixture is diluted with EtOAc and is washed with aqueous NaHCO₃, brine and dried over Na₂SO₄. Removal of the solvents in vacuo provides the title compound.

### Step 26b. Title Compound

The compound of Step 26a is treated with methanol at 25° C. for 24 hours or at refluxing temperature for 24 hours and evaporated to give the title compound.

### Example 27

Compound of formula Ia: A and B taken together with the carbon atom to which they are attached are C═CH₂, L is CH₂CH₃, G is OCH₂C≡CH. Q is N(CH₃)₂, X and Y taken together with the carbon atom to which they are attached are C═O, R₂′ is H, X_{H} is H, R₅ is OH, and R₆is H.

### Step 27a. Compound of formula Ia: A and B taken together with the carbon atom to which they are attached are C═CH₂, L is CH₂CH₃, G is OCH₂C≡CH, Q is N(CH₃)₂, X and Y taken together with the carbon atom to which they are attached are C═O, R₂′ is Ac, X_{H} is H, R₅ is OAc, and R₆ is H

The compound of Step 26a of Example 26 is treated with an excess of phosphonium Wittig reagent according to the literature procedures (a.). Tetrahedron Let., 1999, 40(49), 8575-8578. (b). Synlett., 1996, (6), 521-522.) to provide the title compound.

### Step 27b. Title Compound

The compound of Step 27a is treated with methanol at 25° C. for 24 hours or at refluxing temperature for 2-4 hours and evaporated to provide the title compound.

### Example 28

Compound of formula Ia: A and B taken together with the carbon atom to which they are attached are C═CH₂, L is CH₂CH₃, G is OCH₂C≡C-(3-quinolyl), Q is N(CH₃)₂, X and Y taken together with the carbon atom to which they are attached are C═O, R₂′ is H, X_{H} is H, R₅ is OH, and R₆ is H.

A mixture of a compound of Example 27 (I equivalent) and Pd(PPh₃)₂Cl₂ (0.02 equivalents) in 5:1 acetonitrile:triethylamine is degassed and flushed with nitrogen, treated sequentially with CuI (0.01 equivalents) and 3-bromoquinoline (2-3 equivalents), stirred at room temperature for 10 minutes, heated at 70° C. for 6-24 hours, diluted with ethyl acetate and washed sequentially with water and brine and dried (Na₂SO₄) to provide the title compound.

### Example 29

Compound of formula Ia: A and B taken together with the carbon atom to which they are attached are C═CH₂, L is CH₂CH₃, G is OCH₂CH₂NHCH₂-(4-chlorophenyl), Q is N(CH₃)₂, X and Y taken together with the carbon atom to which they are attached are C═O, R₂′ is H, X_{H} is H, R₅ is OH, and R₆ is H.

To a solution of the compound from Example 26 in methanol is added 4-chlorobenzylamine, excess NaBH₃CN and enough acetic acid to give a pH 4 at room temperature. The reaction mixture is stirred at room temperature for 4-8 hours, cooled to 0° C, neutralized with a solution of saturated aqueous Na₂CO₃ and extracted with CH₂Cl₂. The organic layer is dried over Na₂SO₄, evaporated and purified by column chromatography on silica gel to provide the title compound.

The compounds of Examples 30 through 47 may be prepared according to the procedures described in Examples 22 through 29 and the synthetic schemes and discussions contained herein.

### Example 30

Compound of formula Ia: A and B taken together with the carbon atom to which they are attached are C═CH₂, L is CH₂CH₃, G is OCH₂CH₂NCH₃CH₂-phenyl, Q is N(CH₃)₂, X and Y taken together with the carbon atom to which they are attached are C═O, R₂′ is H, X_{H} is H, R₅ is OH, and R₆ is H.

### Example 31

Compound of formula Ia: A and B taken together with the carbon atom to which they are attached are C═CH₂, L is CH₂CH₃, G is OCH₂CH₂NCH₃CH₂-(2-pyridyl), Q is N(CH₃)₂, X and Y taken together with the carbon atom to which they are attached are C═O, R₂′ is H, X_{H} is H, R₅ is OH, and R₆ is H.

### Example 32

Compound of formula Ia: A and B taken together with the carbon atom to which they are attached are C═CH₂, L is CH₂CH₃, G is OCH₂CH₂N(CH₃)CH₂-(3-pyridyl), Q is N(CH₃)₂, X and Y taken together with the carbon atom to which they are attached are C═O, R₂′ is H, X_{H} is H, R₅ is OH, and R₆ is H.

### Example 33

Compound of formula Ia: A and B taken together with the carbon atom to which they are attached are C═CH₂, L is CH₂CH₃, G is OCH₂CH₂N(CH₃)CH₂-(3-quinolyl), Q is N(CH₃)₂, X and Y taken together with the carbon atom to which they are attached are C═O, R₂′ is H, X_{H} is H, R₅ is OH, and R₆ is H.

### Example 34

Compound of formula Ia: A and B taken together with the carbon atom to which they are attached are C═CH₂, L is CH₂CH₃, G is OCH₂CH═CH-phenyl, Q is N(CH₃)₂, X and Y taken together with the carbon atom to which they are attached are C═O, R₂′ is H, X_{H} is H R₅ is OH, and R₆ is H.

### Example 35

Compound of formula Ia: A and B taken together with the carbon atom to which they are attached are C═CH₂, L is CH₂CH₃, G is OCH₂CH═CH-(2-pyridyl), Q is N(CH₃)₂, X and Y taken together with the carbon atom to which they are attached are C═O, R₂′ is H, X_{H} is H, R₅ is OH, and R₆ is H.

### Example 36

Compound of formula Ia: A and B taken together with the carbon atom to which they are attached are C═CH₂, L is CH₂CH₃, G is OCH₂CH═CH-(3-pyridyl), Q is N(CH₃)₂, X and Y taken together with the carbon atom to which they are attached are C═O, R₂′ is H, X_{H} is H, R₅ is OH, and R₆ is H.

### Example 37

Compound of formula Ia: A and B taken together with the carbon atom to which they are attached are C═CH₂, L is CH₂CH₃, G is OCH₂CH═CH-(3-(5-cyano)pyridyl). Q is N(CH₃)₂, X and Y taken together with the carbon atom to which they are attached are C═O, R₂ ′ is H, X_{H} is H, R₅ is OH, and R₆ is H.

### Example 38

Compound of formula Ia: A and B taken together with the carbon atom to which they are attached are C═CH₂, L is CH₂CH₃, G is OCH₂CH═CH-(6-(aminocarbonyl)-3-quinolyl), Q is N(CH₃)₂, X and Y taken together with the carbon atom to which they are attached are C═O, R₂′ is H, X_{H} is H, R₅ is OH, and R₆ s H.

### Example 39

Compound of formula Ia: A and B taken together with the carbon atom to which they are attached are C═CH₂, L is CH₂CH₃, G is OCH₂C≡C-phenyl, Q is N(CH₃)₂, X and Y taken together with the carbon atom to which they are attached are C═O, R₂′ is H, X_{H} is H, R₅ is OH, and R₆ is H.

### Example 40

Compound of formula Ia: A and B taken together with the carbon atom to which they are attached are C═CH₂, L is CH₂CH₃, G is OCH₂C≡C-(2-pyridyl), Q is N(CH₃)₂, X and Y taken together with the carbon atom to which they are attached are C═O, R₂′ is H, X_{H} is H, R₅ is OH, and R₆ is H.

### Example 41

Compound of formula Ia: A and B taken together with the carbon atom to which they are attached are C═CH₂, L is CH₂CH₃, G is OCH₂C≡C-(3-pyridyl), Q is N(CH₃)₂, X and Y taken together with the carbon atom to which they are attached are C═O, R₂′ is H, X_{H} is H, R₅ is OH, and R₆ is H.

### Example 42

Compound of formula Ia: A and B taken together with the carbon atom to which they are attached are C═CH₂, L is CH₂CH₃, G is OCH₂C≡C-(3-(5-cyano)pyridyl), Q is N(CH₃)₂, X and Y taken together with the carbon atom to which they are attached are C═O, R₂′ is H, X_{H} is H, R₅ is OH, and R₆ is H.

### Example 43

Compound of formula Ia: A and B taken together with the carbon atom to which they are attached are C═CH₂, L is CH₂CH₃, G is OCH₂C≡C-(5-(2-pyridyl)-2-thienyl), Q is N(CH₃)₂, X and Y taken together with the carbon atom to which they are attached are C═O, R₂′ is H, X_{H} is H, R₅ is OH, and R₆ is H.

### Example 44

Compound of formula Ia: A and B taken together with the carbon atom to which they are attached are C═CH₂, L is CH₂CH₃, G is OCH₂C≡C-(5-(3-pyridinyl)-2-pyrrolyl), Q is N(CH₃)₂, X and Y taken together with the carbon atom to which they are attached are C═O, R₂′ is H, X_{H} is H, R₅ is OH, and R₆ is H.

### Example 45

Compound of formula Ia: A and B taken together with the carbon atom to which they are attached are C═CH₂, L is CH₂CH₃, G is OCH₂C≡C-C-(5-(2-pyrimidyl)-2-thienyl), Q is N(CH₃)₂, X and Y taken together with the carbon atom to which they are attached are C═O, R₂′ is H, X_{H} is H, R₅ is OH, and R₆ is H.

### Example 46

Compound of formula Ia: A and B taken together with the carbon atom to which they are attached are C═CH₂, L is CH₂CH₃, G is OCH₂C≡C-(5-(2-pyrazinyl)-2-pyrrolyl), Q is N(CH₃)₂, X and Y taken together with the carbon atom to which they are attached are C═O, R₂′ is H, X_{H} is H, R₅ is OH, and R₆ is H.

### Example 47

Compound of formula Ia: A and B taken together with the carbon atom to which they are attached are C═CH₂, L is CH₂CH₃, G is OCH₂C≡C-C-(6-quinolyl), Q is N(CH₃)₂, X and Y taken together with the carbon atom to which they are attached are C═O, R₂′ is H, X_{H} is H, R₅ is OH, and R₆ is H.

### Example 48

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 48a. Compound 1.2 of Scheme 1: G is OCH₂CH═CH₂, R₁₁ is H, R₂′ is C(O)CH₃ and R₄″ is C(O)CH₃

A mixture of 2′,4″-bis-O-acetyl-6-O-allyl-11-deoxy-10,11-didehydroerythromycin (640 mg, 0.76 mmol), tetrabutylammonium iodide (56 mg, 0.15 mmol), methylene chloride (4.0 mL), propargyl bromide (80% in toluene, 0.68 mL, 6.09 mmol) and sodium hydroxide (50% in water, 6.0 mL) was stirred at room temperature for 2 hours. The mixture was partitioned (ethtyl acetate and water). The organic phases were washed with water and brine, dried (Na₂SO₄) and evaporated. The residue was purified by chromatography (silica, hexanes:acetone/95:5 and 3: 1) to give 258 mg (39%) of the title compound.

MS (ESI) m/z=878 (M+H)⁺.

### Step 48b. Compound 1.4 of Scheme 1: G is OCH₂CH═CH₂, R and R₁₁ taken together with the carbon atom to which they are attached are CHSnBu₃, R₂′ is C(O)CH₃ and R₄″ is C(O)CH₃

A solution of the compound from Step 48a (250 mg, 0.28 mmol) in anhydrous benzene (5.7 mL) was heated to reflux with tributyltin hydride (249 mg, 0.85 mmol) in the presence of AIBN (11.5 mg) for 2 hours before evaporation. The residue was chromatographed (silica, hexanes:acetone/95:5˜9:1) to give the title compound (163.5 mg, 49%).

MS (ESI) m/z=1168/1170 (M+H)⁺.

### Step 48c. Compound 1.6 of Scheme 1: R and R₁₁ taken together with the carbon atom to which they are attached are C═CH₂, G is OCH₂CH═CH₂, and R₂′ is C(O)CH₃

A solution of the compound from Step 1b (163.5 mg, 0.14 mmol) in ethanol (4.0 mL) was treated with hydrochloric acid (2 M, 4.0 mL) at 60° C. for 2 hours before partition (ethyl acetate and saturated NaHCO₃). The organic phases were washed with water and brine, dried (Na₂SO₄) and evaporated. The residue was purified by chromatography (silica, hexanes:acetone/95:5˜4:1) to give the title compound (60.3 mg, 63%).

MS (ESI) m/z=680 (M+H)⁺. ¹³C NMR (125 MHz, CDCl₃): δ 215.7, 174.7, 145.8, 136.4, 116.3, 112.1, 100.1, 86.2, 81.1, 79.7, 77.6, 77.4, 71.6, 70.7, 68.8, 64.6, 63.5, 48.7, 45.4, 44.3, 40.6, 37.3, 36.5, 35.9, 31.3, 22.0, 21.1, 20.4, 19.5, 15.5, 15.3, 14.8, 10.5, 8.1.

### Step 48d. Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is Ac

Into a solution of the compound from Step 48c (60.3 mg, 0.089 mmol) in dichloromethane (3.0 mL) was added Dess-Martin periodinane (56.4 mg, 0.13 mmol) at room temperature. The mixture was stirred at room temperature for 3.5 hours before additional Dess-Martin periodinane (60 mg, 0.14 mmol) was added. The solution was stireed at room temperature for another 1.5 hours and then partitioned with ethyl acetate and saturated sodium bicarbonate-saturated sodium thiosulfate (3:1). The organic phases were washed with water and brine, dried (Na₂SO₄) and evaporated before chromatography (silica, hexanes:acetone/95:5˜85:15) to give the title compound (43.8 mg, 73%).

MS (ESI) m/z=678 (M+H)⁺. ¹³C NMR (125 MHz, CDCl₃): δ 215.4, 206.1, 169.9, 169.7, 145.4, 136.2, 116.4, 112.0, 100.3, 85.9, 78.8, 78.7, 75.1, 71.3, 70.5, 68.9, 64.3, 63.4, 50.6, 48.5, 45.1,44.3, 40.6, 37.6, 36.1, 30.6, 22.1, 21.3, 21.0, 20.3, 19.5, 15.7, 14.8, 12.4, 10.5.

### Step 48e. Title Compound

A solution of the compound from Step 48d (6.5 mg) in methanol (2 mL) was refluxed for 2 hours and then evaporated to give the title compound (6.0 mg, 98%) as one of the CIO stereoisomers.

MS (ESI) m/z=636 (M+H)⁺.

### Example 49

Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 49a. Compound 1.6 of Scheme 1: R and R₁₁ taken together with the carbon atom to which they are attached are C═CH₂, G is OCH₂CH═CH₂, and R₂′ is C(O)CH₃

A solution of the compound from Step 48b (163.5 mg, 0.14 mmol) in ethanol (4.0 mL) was treated with hydrochloric acid (2 M, 4.0 mL) at 60° C. for 2 hours before partition (ethyl acetate and saturated NaHCO₃). The organic phases were washed with water and brine, dried (Na₂SO₄) and evaporated. The residue was purified by chromatography (silica, hexanes:acetone/95:5˜4:1) to give the title compound (24.9 mg, 26%).

MS (ESI) m/z=680 (M+H)⁺. ¹³C NMR (125 MHz, CDCl₃): δ 215.8, 176.7, 152.3, 136.3, 115.2, 107.6, 104.0, 92.2, 86.1, 80.8, 77.2, 72.1, 69.2, 64.7, 64.6, 61.8, 51.1, 48.4, 44.6, 40.8, 39.3, 38.1, 34.8, 29.7, 21.4, 21.2, 20.9, 19.8, 19.6, 15.4, 10.9, 10.7, 8.1.

### Step 49b. Compound of formula I: A and B taken together with the carbon atom to which they are attached are C═CH₂, L is CH₂CH₃, G is OCH₂CH═CH₂, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is Ac

Into a solution of the compound from Step 49a in dichloromethane is added Dess-Martin periodinane at room temperature. The mixture is stirred at room temperature for 3.5 hours before partition with ethyl acetate and saturated sodium bicarbonate—saturated sodium thiosulfate (3:1). The organic phases are washed with water and brine, dried (Na₂SO₄) and evaporated before chromatography to give the title compound as one of the C10 stereoisomers.

### Step 49c. Title Compound

A solution of the compound from Step 49b in methanol is refluxed for 2 hours and then evaporated to give the title compound as one of the C10 stereoisomers.

### Example 50

Compound of formula I: A and B taken together with the carbon atom to which they are attached are C═CH₂, L is CH₂CH₃, G is OCH₂CH═CH-(3-quinolyl), Q is N(CH₃)₂, X and Y taken together with the carbon atom to which they are attached are C═O, U and V taken together with the carbon atom to which they are attached are C═O, Rₑ is H, R_{f} is CH₂, X_{H} is H, and R₂′ is H.

### Step 50a. Compound of formula I: A and B taken together with the carbon atom to which they are attached are C═CH₂, L is CH₂CH₃, G is OCH₂CH═CH-(3-quinolyl), Q is N(CH₃, X and Y taken together with the carbon atom to which they are attached are C═O, U and V taken together with the carbon atom to which they are attached are C═O, Rₑ is H, R_{f} is CH₃, X_{H} is H, and R₂′ is Ac

A mixture of the compound from Step 48d (30.0 mg, 0.044 mmol), tris(o-toluene)phosphine (10.0 mg, 0.033 mmol), palladium acetate (3.0 mg, 0.013 mmol), 3-bromoquinoline (0.015 mL, 0.11 mmol) and triethylamine (0.10 mL, 0.72 mmol) in acetonitrile (1.5 mL) was degassed and warmed to 70° C. The temperature was kept at 70° C. for 0.5 hour before being raised to 100° C. The mixture was kept at this temperature for 16 hours before being evaporated. The residue was further purified by chromatography (silica, hexanes:acetone/95:5˜1.5:1) to give the title compound (12.7 mg, 36%).

MS (ESI) m/z=805 (M+H)⁺.

### Step 50b. Title Compound

A solution of the compound from Step 50a (12.7 mg) in methanol (2 mL) was refluxed for 2 hours and then evaporated. Chromatography (silica, CH₂Cl₂:2M NH₃-MeOH/99:1˜97:3) gave the title compound (8.0 mg, 67%).

MS (ESI) m/z=763 (M+H)⁺.

### Example 51

Compound of formula I: A and B taken together with the carbon atom to which they are attached are C═CH₂, L is CH₂CH₃, G is OCH₂CH(O), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 51a. Compound 1.6 of Scheme 1: R and R₁₁ are each H, G is OCH₂CH(O) and R₂′ is C(O)CH₃

To a solution of the compound of Step 48c of Example 48 in aqueous acetone at 25° C. is added OsO₄ (5 mol %) followed by NaIO₄ (4 equivalents) and the mixture is stirred for 4-6 hours. The reaction mixture is diluted with EtOAc and is washed with aqueous NaHCO₃, brine and dried over Na₂SO₄. Removal of the solvents in vacuo provides the title compound.

### Step 51b. Compound of formula I: A and B taken together with the carbon atom to which they are attached are C═CH₂, L is CH₂CH₃, G is OCH₂CH(O), O is N(CH₃)₂, X and Y taken together with the carbon atom to which they are attached are C═O, U and V taken together with the carbon atom to which they are attached are C═O, Rₑ is H, R_{f} is CH₃, X_{H} and R₂′ is Ac

The compound of Step 51a is treated according to the procedure of Step 48d of Example 48 to provide the title compound.

### Step 51c. Title Compound

The compound of Step 51b is treated with methanol at 25° C. for 24 hours or at refluxing temperature for 2-4 hours. Removal of the solvent provides the title compound.

### Example 52

Compound of formula I: A and B taken together with the carbon atom to which they are attached are C═CH₂, L is CH₂CH₃, G is OCH₂C≡CH, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Step 52a. Compound of formula I: A and B taken together with the carbon atom to which they are attached are C═CH₂, L is CH₂CH₃, G is OCH₂C≡CH, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is Ac

The compound of Step 51a is treated with an excess of phosphonium Wittig reagent according to the literature procedures ((a.). Tetrahedron Lett., 1999, 40(49), 8575-8578. (b). Synlett., 1996, (6), 521-522.) to provide the title compound.

### Step 52b. Title Compound

The compound of Step 52a is treated with methanol at 25° C. for 24 hours or at refluxing temperature for 24 hours. Removal of the solvent provides the title compound.

### Example 53

Compound of formula I: A and B taken together with the carbon atom to which they are attached are C═CH₂, L is CH₂CH₃, G is OCH₂C≡C-(3-quinolyl), Q is N(CH₃)₂, X and Y taken together with the carbon atom to which they are attached are C═O, U and V taken together with the carbon atom to which they are attached are C═O, is H, R_{f} is CH₃, X_{H} is H, and R₂′ is H.

A mixture of a compound of Example 52 (1 equivalent) and Pd(PPh₃)₂Cl₂ (0.02 equivalents) in 5:1/acetonitrile:triethylamine is degassed and flushed with nitrogen, treated sequentially with CuI (0.01 equivalent) and 3-bromoquinoline (2-3 equivalents), stirred at room temperature for 10 minutes, heated at 70° C. for 6-24 hours, diluted with ethyl acetate and washed sequentially with water and brine and dried (Na₂SO₄). Removal of the solvents provides the title compound.

### Example 54

Compound of formula I: A and B taken together with the carbon atom to which they are attached are C═CH₂, L is CH₂CH₃, G is OCH₂CH₂NHCH₂-(4-chlorophenyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

To a solution of the compound of Example 51 in methanol is added 4-chlorobenzylamine, excess NaBH₃CN and enough acetic acid to give a pH 4 at room temperature. The reaction mixture is stirred at room temperature for 4-8 hours. The mixture is cooled to 0° C. and neutralized with a solution of saturated aqueous Na₂CO₃ and extracted with CH₂Cl₂. The organic layer is dried over Na₂SO₄. Removal of the solvents and column chromatography on silica gel provides the title compound.

### Example 55

Compound of formula I: A and B taken together with the carbon atom to which they are attached are C═CH₂, L is CH₂CH₃, G is OCH₂C≡CH, Q is N CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Step 55a. Compound of formula I: A and B taken together with the carbon atom to which they are attached are C═CH₂, L is CH₂CH₃, G is OCH₂C≡CH, Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is Ac

A solution of the compound of Step 52a of Example 5 in DMF at 0° C. is treated with NaH (2 equivalents) and stirred at 0° C. -room temperature for 1 hour followed by addition of (PhSO₂)₂NF (1 equivalent) at 0° C. and is stirred for 2 hours. The reaction mixture is taken up in ethyl acetate and is washed with water, NaHCO₃ and brine and dried over Na₂SO₄. Removal of the solvents provides the title compound.

### Step 55b. Title Compound

The compound of Step 55a is treated with methanol at 25° C. for 24 hours or at refluxing temperature for 24 hours. Removal of the solvent provides the title compound.

### Example 56

Compound of formula I: A and B taken together with the carbon atom to which they are attached are C═CH₂, L is CH₂CH₃, G is OCH₂C≡C-(3-quinolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

A compound of Example 55 is treated according to the procedure of Example 53 to provide the title compound.

Examples 57 through 86 may be prepared according to the procedures described in Examples 50 through 56 and the synthetic schemes and discussions contained herein.

### Example 57

Compound of formula I: A and B taken together with the carbon atom to which they are attached are C═CH₂, L is CH₂CH₃, G is OCH₂CH₂NCH₃CH₂-phenyl, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 58

Compound of formula I: A and B taken together with the carbon atom to which they are attached are C═CH₂, L is CH₂CH₃, G is OCH₂CH₂NCH₃CH₂-(2-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H and R₂′ is H.

### Example 59

Compound of formula I: A and B taken together with the carbon atom to which they are attached are C═CH₂, L is CH₂CH₃, G is OCH₂CH₂NCH₃CH₂-(3-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 60

Compound of formula I: A and B taken together with the carbon atom to which they are attached are C═CH₂, L is CH₂CH₃, G is OCH₂CH₂NCH₃CH₂-(3-quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 61

Compound of formula I: A and B taken together with the carbon atom to which they are attached are C═CH₂, L is CH₂CH₃, G is OCH₂CH₂NCH₃CH₂-(2-pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 62

Compound of formula I: A and B taken together with the carbon atom to which they are attached are C═CH₂, L is CH₂CH₃, G is OCH₂CH₂NCH₃CH₂-(3-quinolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 63

Compound of formula I: A and B taken together with the carbon atom to which they are attached are C═CH₂, L is CH₂CH₃, G is OCH₂CH═CH-phenyl, Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 64

Compound of formula I: A and B taken together with the carbon atom to which they are attached are C═CH₂, L is CH₂CH₃, G is OCH₂CH═CH-(2-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ H.

### Example 65

Compound of formula I: A and B taken together with the carbon atom to which they are attached are C═CH₂, L is CH₂CH₃, G is OCH₂CH═CH-(3-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 66

Compound of formula I: A and B taken together with the carbon atom to which they are attached are C═CH₂, L is CH₂CH₃, G is OCH₂CH═CH-(3-(5-cyano)pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 67

Compound of formula I: A and B taken together with the carbon atom to which they are attached are C═CH₂, L is CH₂CH₃, G is OCH₂CH═CH-(6-(aminocarbonyl)-3-quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H and R₂′ is H.

### Example 68

Compound of formula I: A and B taken together with the carbon atom to which they are attached are C═CH₂, L is CH₂CH₃, G is OCH₂CH═CH-(3:quinolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 69

Compound of formula I: A and B taken together with the carbon atom to which they are attached are C═CH₂, L is CH₂CH₃, G is OCH₂CH═CH-(3-pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 70

Compound of formula I: A and B taken together with the carbon atom to which they are attached are C═CH₂, L is CH₂CH₃, G is OCH₂C≡C-phenyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 71

Compound of formula I: A and B taken together with the carbon atom to which they are attached are C═CH₂, L is CH₂CH₃, G is OCH₂C≡C-(2-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 72

Compound of formula I: A and B taken together with the carbon atom to which they are attached are C═CH₂, L is CH₂CH₃, G is OCH₂C≡C-(3-pyridyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 73

Compound of formula I: A and B taken together with the carbon atom to which they are attached are C═CH₂, L is CH₂CH₃, G is OCH₂C≡C-(3-(5-cyano)pyridyl). Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 74

Compound of formula I: A and B taken together with the carbon atom to which they are attached are C═CH₂, L is CH₂CH₃, G is OCH₂C≡C-(5-(2-pyridyl)-2-thienyl), Q is N(CH₃)₂, X and Y taken together with the carbon atom to which they are attached are C═O, U and V taken together with the carbon atom to which they are attached are C═O, Rₑ is H, R_{f} CH₃, Xₕ is H, and R₂′ H.

### Example 75

Compound of formula I: A and B taken together with the carbon atom to which they are attached are C═CH₂, L is CH₂CH₃, G is OCH₂C≡C-(5-(3-pyridinyl)-2-pyrrolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 76

Compound of formula I: A and B taken together with the carbon atom to which they are attached are C═CH₂, L is CH₂CH₃, G is OCH₂C≡C-(2-pyrimidyl)-2-thienyl), Q is N(CH₃)₂, and Y taken together with the carbon atom to which they are attached are C═O, U and V taken together with the carbon atom to which they are attached are C═O, Rₑ is H, R_{f} is CH₃, X_{H} is H, and R₂′ is H.

### Example 77

Compound of formula I: A and B taken together with the carbon atom to which they are attached are C═CH₂, L is CH₂CH₃, G is OCH₂C≡C-(5-(2-pyrazinyl)-2-pyrrolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 78

Compound of formula I: A and B taken together with the carbon atom to which they are attached are C═CH₇, L is CH₂CH₃, G is OCH₂C═C-(6-quinolyl), Q is N(CH₃)₂, X and Y taken together with the carbon atom to which they are attached are C═O, U and V taken together with the carbon atom to which they are attached are C═O, Rₑ is H, R_{f} is CH₃, X_{H} is H, and R₂′ is H.

### Example 79

Compound of formula I: A and B taken together with the carbon atom to which they are attached are C═CH₂, L is CH₂CH₃, G is OCH₂C≡C-phenyl, Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 80

Compound of formula I: A and B taken together with the carbon atom to which they are attached are C═CH₂, L is CH₂CH₃, G is OCH₂C≡C-(2-pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 81

Compound of formula I: A and B taken together with the carbon atom to which they are attached are C═CH₂, L is CH₂CH₃ , G is OCH₂C≡C-(3-pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 82

Compound of formula I: A and B taken together with the carbon atom to which they are attached are C═CH₂, L is CH₂CH₃, G is OCH₂C≡C-(3-(5-cyano)pyridyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 83

Compound of formula I: A and B taken together with the carbon atom to which they are attached are C═CH₂, L is CH₂CH₃, G is OCH₂C≡C-(5-(2-pyridyl)-2-thienyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 84

Compound of formula I: A and B taken together with the carbon atom to which they are attached are C═CH₂, L is CH₂CH₃, G is OCH₂C≡C-(5-(2-pyrimidyl)-2-thienyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 85

Compound of formula I: A and B taken together with the carbon atom to which they are attached are C═CH₂, L is CH₂CH₃, G is OCH₂C≡C-(5-(2-pyridinyl)-2-pyrrolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

### Example 86

Compound of formula I: A and B taken together with the carbon atom to which they are attached are C═CH₂, L is CH₂CH₃, G is OCH₂C≡C-(5-(2-pyrazinyl)-2-pyrrolyl), Q is N(CH₃)₂, X and Y taken together with the carbon atom to which they are attached are C═O, U and V taken together with the carbon atom to which they are attached are C═O, Rₑ is F, R_{f} is CH₃, X_{H} is H, and R₂′ is H.

Although the invention has been described in detail with respect to various preferred embodiments it is not intended to be limited thereto, but rather those skilled in the art will recognize that variations and modifications may be made therein which are within the spirit of the invention and the scope of the appended claims.

What is claimed is:

1. A compound of formula (I): 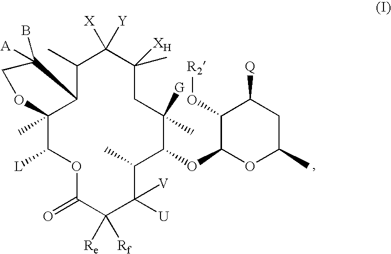
or their racemates, enantiomers, regioisomers, salts, esters or prodrugs thereof, wherein
A and B are independently selected from: halogen, NO₂, —CN, R₁, OR₁, S(O)ₙR₁, —NR₁C(O)R₂, —NR₁C(O)NR₃R₄, —NHS(O)ₙR₁, —C(O)NR₃R₄, —OC(O)NR₃R₄ and NR₃R₄;
Each R₁ and R₂ is independently selected from: hydrogen, deuterium, acyl, silane, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group;
Each of R₃ and R₄ is independently selected from: hydrogen, acyl, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, a substituted or unsubstituted heterocyclic group; or can be taken together with the nitrogen atom to which they are attached to form a substituted or unsubstituted heterocyclic or heteroaromatic ring;
or A and B, taken together with the carbon atom to which they are attached, form a substituted or unsubstituted alicyclic, aromatic, heterocyclic or heteroaromatic ring;
or A and B, taken together with the carbon atom to which they are attached, are selected from: CO, C═CR₁R₂, C═NR₁, C═NOR₁, C═NO(CH₂)ₘR₁, C═NNHR₁, C═NNHCOR₁, C═NNHCONR₃R₄, C═NNHS(O)ₙR₁, or C═N—N═CR₁R₂;
L is selected from hydrogen, a substituted or unsubstituted, saturated or unsaturated aliphatic group, a substituted or unsubstituted, saturated or unsaturated alicyclic group, a substituted or unsubstituted aromatic group, a substituted or unsubstituted heteroaromatic group, or a substituted or unsubstituted heterocyclic group;
G is independently selected from hydrogen, —CN or OR₁;
one of U or V is hydrogen and the other is independently selected from: R₁, OR₁, OC(O)R₁, OC(O)NR₃R₄, S(O)ₙR₁, 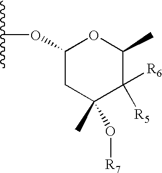
or other carbohydrate or sugar moiety;
one of R₅ or R₆ is hydrogen and the other is selected from: R₁, OR₁, or NR₃R₄;
or R₅ and R₆, taken together with the carbon atom to which they are attached, are selected from: C═O, C═C(R₁)₂, C═NR₁, C═C(R₁)₂, C═NOR₁, C═NO(CH₂)ₘR₁, C═NNR₃R₄, C═NNHCOR₁, C═NNHCONR₃R₄, C═NNHS(O)ₙR₁, or C═N—N═C(R₁)₂;
R₇ is independently selected from hydrogen or methyl;
or U and V, taken together with the carbon atom to which they are attached, are C═O;
or UV and RₑR_{f}, taken together with the carbon atoms to which they are attached, are —C(R₁)═CH—;
one of Rₑ and R_{f} is selected from hydrogen or methyl, and the other is independently selected from halogen, deuterium, or R₁.
Q is NR₃R₄;
one of X and Y is hydrogen, substituted or unsubstituted aliphatic, and the other is independently selected from: hydroxy, —SH, —NH₂, or —NR₁H;
or X and Y, taken together with the carbon atom to which they are attached, are selected from: C═O, C═C(R₁)₂, C═NR₁, C═NOR₁, C═NO(CH₂)ₘR₁, C═NNHR₁, C═NNHCOR₁, C═NNHCONR₃R₄, C═NNHS(O)ₙR₁, or C═N—N═C(R₁)₂;
R₂′ and Rₚ are independently selected from hydrogen or a hydroxy protecting group;
X_{H} is selected from hydrogen or halogen;
m is an integer; and
n is 0, 1, or 2.
